(19) United States
(12) Patent Application Publication
(10) Pub. No.: US 2014/0080894 A1
(43) Pub. Date: Mar. 20, 2014

(54) ENHANCED BIODISTRIBUTION OF OLIGOMERS

(71) Applicant: David L. Mcelligott, Bothell, WA (US)

(72) Inventor: David L. Mcelligott, Bothell, WA (US)

(21) Appl. No.: 14/002,648

(22) PCT Filed: Mar. 2, 2012
(86) PCT No.: PCT/US12/27431

§ 371 Date: Nov. 25, 2013

Related U.S. Application Data
(60) Provisional application No. 61/448,618, filed on Mar. 2, 2011.

(51) Int. Cl. C12N 15/113

(52) U.S. Cl. CPC C12N 15/113
USPC 514/44 A; 435/375

(57) ABSTRACT

This invention relates generally to oligomers useful for modulating expression and/or activity of RNAs or genes. More particularly, the invention relates to single stranded, double stranded, partially double stranded and hairpin structured chemically modified oligomers that have a broad biodistribution profile for inhibiting RNA expression in a plurality of cells, tissues, or organs, and to methods of making and using the modified oligomers.

### CROSS REFERENCE TO RELATED APPLICATIONS

This application claims the benefit under 35 U.S.C. §119(e) of U.S. Provisional Application No. 61/448,618, filed Mar. 2, 2011, which is incorporated by reference in its entirety.

### STATEMENT REGARDING THE SEQUENCE LISTING

The Sequence Listing associated with this application is provided in text format in lieu of a paper copy, and is hereby incorporated by reference into the specification. The name of the text file containing the Sequence Listing is GROO_{—}001_{—}01WO_ST25.txt. The text file is 4 KB, was created on Mar. 2, 2012, and is being submitted electronically via EFS-Web.

### BACKGROUND

1. Technical Field

This invention relates generally to oligomers useful for modulating gene expression in a wide variety of tissues. More particularly, the invention relates to discovering that oligomers conjugated to one or more minor groove binders having advantageous biodistribution properties compared to existing oligomers.

2. Description of the Related Art

The human genome comprises vast amounts of genetic regulation. Regulation exists at the level of transcription, post-transcriptionally, translationally, and post-translationally. Recently, scientists have sought to regulate gene expression via RNA interference. RNA interference refers to the process of sequence-specific post-transcriptional gene silencing in animals mediated by the RNA interference pathway (RNAi). Synthetic duplex oligonucleotides, such as short interfering RNAs (siRNAs), small hairpin RNAs (shRNAs), Piwi-interacting RNA (piRNA) mimics, and microRNA (miRNA) mimics have been used to successfully modulate gene expression, leading to the cleavage of specific messenger RNAs (mRNAs).

There are also a number of epigenetic modifiers that play a role in regulating the genome. It was thought that most post-transcriptional regulation was mediated by the presence or absence of stabilizing sequences in the 3′ untranslated regions of genes. However, recently scientists have discovered a new class of small, non-coding RNAs termed “microRNA.”

MicroRNAs are a family of small, non-coding RNAs that regulate gene expression in a sequence-specific manner. The two founding members of the microRNA family were originally identified in Caenorhabditis elegans as genes that were required for the timed regulation of developmental events. Since then, hundreds of microRNAs have been identified in almost all metazoan genomes, including worms, flies, plants and mammals. MicroRNAs have diverse expression patterns and regulate various developmental and physiological processes. A single miRNA or cluster of miRNAs can regulate genes associated with developmental processes, cell metabolism and homeostasis, and cell proliferation, growth and cell death. Deregulation of a miRNA can lead to various pathological conditions, including obesity, cancer, heart disease, neurodegenerative diseases, and degenerative musculoskeletal disease.

Thus, miRNAs are an attractive drug target. However, little progress has been made in overcoming the poor biodistribution problems associated with systemic administration of candidate drugs such as anti-miRNA molecules, siRNAs, shRNAs, piRNA mimetics, and miRNA mimetics. In addition to the limited biodistribution, anti-miRNA molecules, siRNAs, shRNAs, piRNA mimetics, and miRNA mimetics can have deleterious “off-target” effects, meaning that they not only down-regulate the pathologically gene expression but also likely inhibit gene expression that regulates other essential and beneficial cellular processes. Thus, these molecules are not yet ready for mainstream use in the clinic.

Accordingly, there exists a pressing need in the art to develop anti-miRNA, sRNA, shRNA, piRNA mimetic, and miRNA mimetic compounds that are more widely biodistributed; that have increased bioavailability; and that are highly effective in regulating gene expression.

The methods and compositions of the present invention provide solutions to these and other problems in the art.

### BRIEF SUMMARY

The invention generally relates to oligomer compounds that have improved biodistribution properties and methods of using the same.

In one embodiment, the present invention contemplates, in part, a method of providing an oligomer to a plurality of cell types, tissues, or organs comprising administering to a subject, an oligomer comprising one or more minor groove binding moieties (MGBs).

In another embodiment, the present invention contemplates, in part, a method of administering an oligomer to a plurality of cell types, tissues, or organs comprising administering to a subject, an oligomer comprising one or more MGBs.

In various embodiments, the oligomer is a single stranded oligonucleotide. In various other embodiments, the oligomer is a double stranded oligonucleotide. In various particular embodiments, the oligomer is selected from the group consisting of: an anti-miRNA, an sRNA, an shRNA, a piRNA mimetic, and a miRNA mimetic. In particular embodiments, the oligomer is parenterally administered.

In certain embodiments, parenteral administration is selected from the group consisting of: intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

In certain particular embodiments, the oligomer is intravenously administered.

In further embodiments, the plurality of cell types is selected from the group consisting of: cancer cells, immune cells, epithelial cells, endothelial cells, mesodermal cells, and mesenchymal cells, bone cells, hematopoietic cells, skin cells, hair cells, eye cells, neural cells, glial cells, muscle cells, meningeal cells, breast cells, liver cells, kidney cells, pancreatic cells, gastric cells, intestinal cells, colon cells, prostate cells, cervical cells, and vaginal cells.

In additional embodiments, the plurality of tissues is selected from the group consisting of: mesodermal tissue, connective tissue, smooth muscle tissue, striated muscle tissue, cardiac muscle tissue, bone tissue, bone marrow tissue, bone spongy tissue, cartilage tissue, adipose tissue, endodermal tissue, lung tissue, vascular tissue, pancreatic tissue, liver tissue, pancreatic ductal tissue, spleen tissue, thymus tissue, tonsil tissue, Peyer's patch tissue, lymph nodes tissue, thyroid tissue, endothelial tissue, blood cells, bladder tissue, kidney tissue, digestive tract tissue, esophagus tissue, stomach tissue, small intestine tissue, large intestine tissue, uterus tissue, testicular tissue, ovarian tissue, prostate tissue, endocrine tissue, mesentery tissue, and umbilical tissue, ectodermal tissue, epidermis tissue, dermis tissue, eye tissue, and nervous system tissue.

In particular embodiments, the plurality of organs is selected from the group consisting of: bladder, bone, brain, breast, cartilage, cervix, colon, cornea, eye, nervous tissue, glia, esophagus, fallopian tube, heart, pancreas, intestines, gallbladder, kidney, liver, lung, ovaries, pancreas, parathyroid gland, pineal gland, pituitary gland, prostate, spinal cord, spleen, skeletal muscle, skin, smooth muscle, stomach, testes, thymus, thyroid, trachea, urogenital tract, ureter, urethra, uterus, and vagina.

In some embodiments, the oligomer hybridizes to a pre-mRNA. In another embodiment, the oligomer hybridizes to a pre-mRNA comprising a target miRNA.

In another embodiment, the oligomer hybridizes to a pre-mRNA comprising a target pri-miRNA.

In certain embodiments, at least one of the one or more MGBs is conjugated to the 5′ end or the 3′ end of the oligomer.

In certain particular embodiments, at least one of the one or more MGBs is conjugated to the oligomer with a linker.

In additional embodiments, the linker comprises a chain of about 10 to about 100 atoms selected from the group consisting of: C, O, N, S, and P.

In particular embodiments, the linker is selected from the group consisting of: a) —P(═O)(OH)O(CH2)6NH—; b) —P(═O)(OH)O(CH2)4NH—; c) —P(═O)(OH)(OCH2CH2)6OP(═O)(OH)O(CH2)6NH—; d) hydroxy{[5-(hydroxymethyl)-1-methylpyrrolidin-3 yl]oxy}oxophosphonium; and e) —(CH2)5OP(═O)(OH)—.

In various embodiments, at least one of the one or more MGBs selected from the group consisting of: netropsin, distamycin and lexitropsin, mithramycin, chromomycin A3, olivomycin, anthramycin, sibiromycin, 1,2-dihydro-3H-pyrrolo[3,2-e)indole-7-carboxylic acid (DPI)₍₁₋₁₀₎, N3 carbamoyl 1,2-dihydro-3H-pyrrolo[3,2-e)indole-7-carboxylic acid (CDPI)₍₁₋₁₀₎, and N-methylpyrrole-4-carbox-2-amide (MPC)₍₁₋₁₀₎.

In particular embodiments, at least one of the one or more MGBs is CDPI₃ or CDPI₄.

In certain embodiments, the at least one of the one or more MGBs is CDPI₃.

In further embodiments, the oligomer comprises 6 to 100 nucleotides.

In further particular embodiments, the oligomer comprises 10 to 50 nucleotides.

In further certain embodiments, the oligomer comprises 15 to 23 nucleotides.

In additional embodiments, the oligomer comprises a nucleotide sequence that is at least 70% complementary to a target sequence. In one embodiment, the target sequence is an mRNA, in another embodiment, the target sequence is an miRNA sequence.

In some embodiments, the nucleotides are selected from the group of deoxyribonucleotides, ribonucleotides, and modified nucleotides.

In some particular embodiments, the modified nucleotides comprise a base selected from the groups consisting of: 5-methylcytosine, 5-hydroxymethyl cytosine, xanthine, hypoxanthine, 2-aminoadenine, 6-alkyladenine, 6-alkylguanine, 2-alkyladenine, 2-alkylguanine, 2-thiouracil, 2-thiothymine, 2-thiocytosine, 5-halouracil, 5-halocytosine, 5-alkynyluracil, 5-alkynylcytosine, 6-azo uracil, 6-azo cytosine, 6-azo thymine, 5-uracil, 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halo uracil, 5-halo cytosine, 7-methylguanine, 7-methyladenine, 2-F-adenine, 2-amino-adenine, 8-azaguanine, 8azaadenine, 7-deazaguanine, 7-deazaadenine, 3-deazaguanine, 3-deazaadenine, phenoxazine cytidine, phenothiazine cytidine, G-clamp, carbazole cytidine, pyridoindole cytidine, 7-deaza adenine, 7-deaza guanosine, 2-aminopyridine, 2-pyridone, 2-aminopropyladenine, 5-propynyluracil, and 5-propynylcytosine.

In particular embodiments, the oligomer comprises at least one modified internucleoside linkage.

In certain particular embodiments, the at least one modified internucleoside linkage is selected from the group consisting of: a phosphorothioate, phosphorodithioate, phosphotriester, aminoalkylphosphotriester, alkyl phosphonates, phosphinate, phosphoramidate, thionophosphoramidate, thionoalkylphosphonate, thionoalkylphosphotriester, selenophosphate, boranophosphate, morpholino, siloxane, sulfide, sulfoxide, sulfone, formacetyl, thioformacetyl, methylene formacetyl, riboacetyl, alkene-containing backbone, sulfamate, methyleneimino, methylenehydrazino, sulfonate, sulfonamide, or amide.

In additional particular embodiments, the at least one modified internucleoside linkage is a phosphorothioate linkage.

In additional certain embodiments, all of the internucleoside linkages of the oligomer are phosphorothioate linkages.

In particular embodiments, the oligomer comprises at least one 2′ modified sugar moiety.

In certain embodiments, the at least one 2′ modified sugar moiety is selected from the group consisting of: OH, halogen, O-alkyl, S-alkyl, N-alkyl, O-alkenyl, S-alkenyl, N-alkenyl, O-alkynyl, S-alkynyl, N-alkynyl, O-alkyl-O-alkyl, alkaryl, aralkyl, O-alkaryl, O-aralkyl, SH, SCH3, OCN, CN, CF3, OCF3, SOCH3, SO2CH3, ONO2, NO2, N3, NH2, heterocycloalkyl, heterocycloalkaryl, aminoalkylamino, polyalkylamino, substituted silyl, alkoxyalkoxy, dimethylaminooxyethoxy, allyl, and O-allyl, wherein the alkyl, alkenyl and alkynyl can be substituted or unsubstituted C1 to C10 alkyl or C2 to C10 alkenyl and alkynyl.

In additional embodiments, the at least one 2′ modified sugar moiety is 2′-O-(2-methoxyethyl) (2′-MOE) sugar moiety.

In related additional embodiments, the at least one 2′ modified sugar moiety comprises a 2′-O, 4′-C methylene bridge.

In further embodiments, the oligomer comprises at least one or more bases comprising a 3′ lipophilic group.

In particular embodiments, the 3′ lipophilic group is selected from the group consisting of: cholesterol; a bile acid; and a fatty acid.

In one embodiment, the present invention contemplates, in part, a method of decreasing the expression of one or more genes in one or more cells, tissues, or organs, comprising administering an oligomer comprising one or more MGBs to a subject, wherein the gene expression of the one or more genes in the subject is decreased in the one or more cells, tissues, or organs, compared to the gene expression in another subject administered an oligomer that does not comprise an MGB.

In one embodiment, the present invention contemplates, in part, a method of decreasing the miRNA activity of an miRNA in one or more cells, tissues, or organs, comprising administering an oligomer comprising one or more MGBs to a subject, wherein the miRNA activity in the subject is decreased in the one or more cells, tissues, or organs, compared to the miRNA activity in another subject administered an oligomer that does not comprise an MGB.

In another embodiment, the present invention contemplates, in part, a method of treating a subject having a disease, disorder or condition associated with increased gene expression of one or more genes in a plurality of cell types, tissues, or organs comprising: a) identifying one or more genes in a plurality of cell types, tissues, or organs having increased gene expression in diseased cells, tissues, or organs, compared to gene expression of the one or more genes in a normal cell; and b) administering an oligomer comprising one or more MGBs that hybridizes to the one or more genes.

In another embodiment, the present invention contemplates, in part, a method of treating a subject having a disease, disorder or condition associated with increased activity of one or more miRNAs in a plurality of cell types, tissues, or organs comprising: a) identifying one or more miRNAs in a plurality of cell types, tissues, or organs having increased miRNA activity in diseased cells, tissues, or organs, compared to miRNA activity of the one or more miRNA in a normal cell; and b) administering an oligomer comprising one or more MGBs that hybridizes to the one or more miRNAs.

In particular embodiments, the disease, disorder or condition is selected from the groups consisting of: tumor mediated angiogenesis, cancer, inflammation, fibrotic diseases, auto-immune diseases, and hepatitis C infection-mediated diseases.

In additional particular embodiments, the disease, disorder or condition is selected from the groups consisting of: lung cancer, hepatocellular cancer, gastrointestinal cancer, pancreatic cancer, glioblastoma, leukemia, lymphoma, cervical cancer, ovarian cancer, kidney cancer, bladder cancer, breast cancer, osteosarcoma, cancer of the central nervous system, colon cancer, colorectal cancer, gastric cancer, endometrial or uterine carcinoma, salivary gland carcinoma, papillary renal cell carcinoma, prostate cancer, vulval cancer, thyroid cancer, and head and neck cancer, and melanoma.

In further particular embodiments, the disease, disorder or condition is selected from the groups consisting of: autoimmune thyroid disease, including Grave's disease and Hashimoto's thyroiditis, rheumatoid arthritis, systemic lupus erythematosus (SLE), Sjogrens syndrome, immune thrombocytopenic purpura (ITP), multiple sclerosis (MS), myasthenia gravis (MG), psoriasis, scleroderma, and inflammatory bowel disease, including Crohn's disease and ulcerative colitis.

In certain particular embodiments, the disease, disorder or condition Hepatitis C infection or a Hepatitis C infection-mediated disease.

In certain other particular embodiments, the disease, disorder or condition is selected from the groups consisting of: neovascularization, stroke, ischemia, and myocardial infarction.

In various embodiments, the miRNA is selected from the group consisting of: hsa-let-7a-1; hsa-let-7a-2; hsa-let-7a-3; hsa-let-7b; hsa-let-7c; hsa-let-7d; hsa-let-7e; hsa-let-7f-1; hsa-let-7f-2; hsa-let-7g; hsa-let-7i; hsa-mir-100; hsa-mir-101-1; hsa-mir-101-2; hsa-mir-103-1; hsa-mir-103-1-as; hsa-mir-103-2; hsa-mir-103-2-as; hsa-mir-105-1; hsa-mir-105-2; hsa-mir-106a; hsa-mir-106b; hsa-mir-107; hsa-mir-10a; hsa-mir-10b; hsa-mir-1-1; hsa-mir-1178; hsa-mir-1179; hsa-mir-1180; hsa-mir-1181; hsa-mir-1182; hsa-mir-1183; hsa-mir-1184-1; hsa-mir-1184-2; hsa-mir-1184-3; hsa-mir-1185-1; hsa-mir-1185-2; hsa-mir-1193; hsa-mir-1197; hsa-mir-1-2; hsa-mir-1200; hsa-mir-1202; hsa-mir-1203; hsa-mir-1204; hsa-mir-1205; hsa-mir-1206; hsa-mir-1207; hsa-mir-1208; hsa-mir-122; hsa-mir-1224; hsa-mir-1225; hsa-mir-1226; hsa-mir-1227; hsa-mir-1228; hsa-mir-1229; hsa-mir-1231; hsa-mir-1233-1; hsa-mir-1233-2; hsa-mir-1234; hsa-mir-1236; hsa-mir-1237; hsa-mir-1238; hsa-mir-124-1; hsa-mir-124-2; hsa-mir-1243; hsa-mir-124-3; hsa-mir-1244-1; hsa-mir-1244-2; hsa-mir-1244-3; hsa-mir-1245; hsa-mir-1246; hsa-mir-1247; hsa-mir-1248; hsa-mir-1249; hsa-mir-1250; hsa-mir-1251; hsa-mir-1252; hsa-mir-1253; hsa-mir-1254; hsa-mir-1255a; hsa-mir-1255b-1; hsa-mir-1255b-2; hsa-mir-1256; hsa-mir-1257; hsa-mir-1258; hsa-mir-125a; hsa-mir-125b-1; hsa-mir-125b-2; hsa-mir-126; hsa-mir-1260; hsa-mir-1260b; hsa-mir-1261; hsa-mir-1262; hsa-mir-1263; hsa-mir-1264; hsa-mir-1265; hsa-mir-1266; hsa-mir-1267; hsa-mir-1268; hsa-mir-1269; hsa-mir-127; hsa-mir-1270-1; hsa-mir-1270-2; hsa-mir-1271; hsa-mir-1272; hsa-mir-1273; hsa-mir-1273c; hsa-mir-1273d; hsa-mir-1274a; hsa-mir-1274b; hsa-mir-1275; hsa-mir-1276; hsa-mir-1277; hsa-mir-1278; hsa-mir-1279; hsa-mir-1280; hsa-mir-1281; hsa-mir-128-1; hsa-mir-1282; hsa-mir-128-2; hsa-mir-1283-1; hsa-mir-1283-2; hsa-mir-1284; hsa-mir-1285-1; hsa-mir-1285-2; hsa-mir-1286; hsa-mir-1287; hsa-mir-1288; hsa-mir-1289-1; hsa-mir-1289-2; hsa-mir-1290; hsa-mir-1291; hsa-mir-129-1; hsa-mir-1292; hsa-mir-129-2; hsa-mir-1293; hsa-mir-1294; hsa-mir-1295; hsa-mir-1296; hsa-mir-1297; hsa-mir-1298; hsa-mir-1299; hsa-mir-1301; hsa-mir-1302-1; hsa-mir-1302-10; hsa-mir-1302-11; hsa-mir-1302-2; hsa-mir-1302-3; hsa-mir-1302-4; hsa-mir-1302-5; hsa-mir-1302-6; hsa-mir-1302-7; hsa-mir-1302-8; hsa-mir-1302-9; hsa-mir-1303; hsa-mir-1304; hsa-mir-1305; hsa-mir-1306; hsa-mir-1307; hsa-mir-130a; hsa-mir-130b; hsa-mir-132; hsa-mir-1321; hsa-mir-1322; hsa-mir-1323; hsa-mir-1324; hsa-mir-133a-1; hsa-mir-133a-2; hsa-mir-133b; hsa-mir-134; hsa-mir-135a-1; hsa-mir-135a-2; hsa-mir-135b; hsa-mir-136; hsa-mir-137; hsa-mir-138-1; hsa-mir-138-2; hsa-mir-139; hsa-mir-140; hsa-mir-141; hsa-mir-142; hsa-mir-143; hsa-mir-144; hsa-mir-145; hsa-mir-1468; hsa-mir-1469; hsa-mir-146a; hsa-mir-146b; hsa-mir-147; hsa-mir-1470; hsa-mir-1471; hsa-mir-147b; hsa-mir-148a; hsa-mir-148b; hsa-mir-149; hsa-mir-150; hsa-mir-151; hsa-mir-152; hsa-mir-153-1; hsa-mir-153-2; hsa-mir-1537; hsa-mir-1538; hsa-mir-1539; hsa-mir-154; hsa-mir-155; hsa-mir-15a; hsa-mir-15b; hsa-mir-16-1; hsa-mir-16-2; hsa-mir-17; hsa-mir-181a-1; hsa-mir-181a-2; hsa-mir-181b-1; hsa-mir-181b-2; hsa-mir-181c; hsa-mir-181d; hsa-mir-182; hsa-mir-1825; hsa-mir-1827; hsa-mir-183; hsa-mir-184; hsa-mir-185; hsa-mir-186; hsa-mir-187; hsa-mir-188; hsa-mir-18a; hsa-mir-18b; hsa-mir-190; hsa-mir-1908; hsa-mir-1909; hsa-mir-190b; hsa-mir-191; hsa-mir-1910; hsa-mir-1911; hsa-mir-1912; hsa-mir-1913; hsa-mir-1914; hsa-mir-1915; hsa-mir-192; hsa-mir-193a; hsa-mir-193b; hsa-mir-194-1; hsa-mir-194-2; hsa-mir-195; hsa-mir-196a-1; hsa-mir-196a-2; hsa-mir-196b; hsa-mir-197; hsa-mir-1972-1; hsa-mir-1972-2; hsa-mir-1973; hsa-mir-1976; hsa-mir-198; hsa-mir-199a-1; hsa-mir-199a-2; hsa-mir-199b; hsa-mir-19a; hsa-mir-19b-1; hsa-mir-19b-2; hsa-mir-200a; hsa-mir-200b; hsa-mir-200c; hsa-mir-202; hsa-mir-203; hsa-mir-204; hsa-mir-205; hsa-mir-2052; hsa-mir-2053; hsa-mir-2054; hsa-mir-206; hsa-mir-208a; hsa-mir-208b; hsa-mir-20a; hsa-mir-20b; hsa-mir-21; hsa-mir-210; hsa-mir-211; hsa-mir-2110; hsa-mir-2113; hsa-mir-2114; hsa-mir-2115; hsa-mir-2116; hsa-mir-2117; hsa-mir-212; hsa-mir-214; hsa-mir-215; hsa-mir-216a; hsa-mir-216b; hsa-mir-217; hsa-mir-218-1; hsa-mir-218-2; hsa-mir-219-1; hsa-mir-219-2; hsa-mir-22; hsa-mir-221; hsa-mir-222; hsa-mir-223; hsa-mir-224; hsa-mir-2276; hsa-mir-2277; hsa-mir-2278; hsa-mir-2355; hsa-mir-23a; hsa-mir-23b; hsa-mir-23c; hsa-mir-24-1; hsa-mir-24-2; hsa-mir-25; hsa-mir-26a-1; hsa-mir-26a-2; hsa-mir-26b; hsa-mir-27a; hsa-mir-27b; hsa-mir-28; hsa-mir-2861; hsa-mir-2909; hsa-mir-296; hsa-mir-297; hsa-mir-298; hsa-mir-299; hsa-mir-29a; hsa-mir-29b-1; hsa-mir-29b-2; hsa-mir-29c; hsa-mir-300; hsa-mir-301a; hsa-mir-301b; hsa-mir-302a; hsa-mir-302b; hsa-mir-302c; hsa-mir-302d; hsa-mir-302e; hsa-mir-302f; hsa-mir-3065; hsa-mir-3074; hsa-mir-30a; hsa-mir-30b; hsa-mir-30c-1; hsa-mir-30c-2; hsa-mir-30d; hsa-mir-30e; hsa-mir-31; hsa-mir-3115; hsa-mir-3116-1; hsa-mir-3116-2; hsa-mir-3117; hsa-mir-3118-1; hsa-mir-3118-2; hsa-mir-3118-3; hsa-mir-3118-4; hsa-mir-3118-5; hsa-mir-3118-6; hsa-mir-3119-1; hsa-mir-3119-2; hsa-mir-3120; hsa-mir-3121; hsa-mir-3122; hsa-mir-3123; hsa-mir-3124; hsa-mir-3125; hsa-mir-3126; hsa-mir-3127; hsa-mir-3128; hsa-mir-3129; hsa-mir-3130-1; hsa-mir-3130-2; hsa-mir-3131; hsa-mir-3132; hsa-mir-3133; hsa-mir-3134; hsa-mir-3135; hsa-mir-3136; hsa-mir-3137; hsa-mir-3138; hsa-mir-3139; hsa-mir-3140; hsa-mir-3141; hsa-mir-3142; hsa-mir-3143; hsa-mir-3144; hsa-mir-3145; hsa-mir-3146; hsa-mir-3147; hsa-mir-3148; hsa-mir-3149; hsa-mir-3150; hsa-mir-3150b; hsa-mir-3151; hsa-mir-3152; hsa-mir-3153; hsa-mir-3154; hsa-mir-3155; hsa-mir-3156-1; hsa-mir-3156-2; hsa-mir-3156-3; hsa-mir-3157; hsa-mir-3158-1; hsa-mir-3158-2; hsa-mir-3159; hsa-mir-3160-1; hsa-mir-3160-2; hsa-mir-3161; hsa-mir-3162; hsa-mir-3163; hsa-mir-3164; hsa-mir-3165; hsa-mir-3166; hsa-mir-3167; hsa-mir-3168; hsa-mir-3169; hsa-mir-3170; hsa-mir-3171; hsa-mir-3173; hsa-mir-3174; hsa-mir-3175; hsa-mir-3176; hsa-mir-3177; hsa-mir-3178; hsa-mir-3179-1; hsa-mir-3179-2; hsa-mir-3179-3; hsa-mir-3180-1; hsa-mir-3180-2; hsa-mir-3180-3; hsa-mir-3180-4; hsa-mir-3180-5; hsa-mir-3181; hsa-mir-3182; hsa-mir-3183; hsa-mir-3184; hsa-mir-3185; hsa-mir-3186; hsa-mir-3187; hsa-mir-3188; hsa-mir-3189; hsa-mir-3190; hsa-mir-3191; hsa-mir-3192; hsa-mir-3193; hsa-mir-3194; hsa-mir-3195; hsa-mir-3196; hsa-mir-3197; hsa-mir-3198; hsa-mir-3199-1; hsa-mir-3199-2; hsa-mir-32; hsa-mir-3200; hsa-mir-3201; hsa-mir-3202-1; hsa-mir-3202-2; hsa-mir-320a; hsa-mir-320b-1; hsa-mir-320b-2; hsa-mir-320c-1; hsa-mir-320c-2; hsa-mir-320d-1; hsa-mir-320d-2; hsa-mir-320e; hsa-mir-323; hsa-mir-323b; hsa-mir-324; hsa-mir-325; hsa-mir-326; hsa-mir-328; hsa-mir-329-1; hsa-mir-329-2; hsa-mir-330; hsa-mir-331; hsa-mir-335; hsa-mir-337; hsa-mir-338; hsa-mir-339; hsa-mir-33a; hsa-mir-33b; hsa-mir-340; hsa-mir-342; hsa-mir-345; hsa-mir-346; hsa-mir-34a; hsa-mir-34b; hsa-mir-34c; hsa-mir-3605; hsa-mir-3606; hsa-mir-3607; hsa-mir-3609; hsa-mir-361; hsa-mir-3610; hsa-mir-3611; hsa-mir-3612; hsa-mir-3613; hsa-mir-3614; hsa-mir-3615; hsa-mir-3616; hsa-mir-3617; hsa-mir-3618; hsa-mir-3619; hsa-mir-362; hsa-mir-3620; hsa-mir-3621; hsa-mir-3622a; hsa-mir-3622b; hsa-mir-363; hsa-mir-3646; hsa-mir-3647; hsa-mir-3648; hsa-mir-3649; hsa-mir-3650; hsa-mir-3651; hsa-mir-365-1; hsa-mir-3652; hsa-mir-365-2; hsa-mir-3653; hsa-mir-3654; hsa-mir-3655; hsa-mir-3656; hsa-mir-3657; hsa-mir-3658; hsa-mir-3659; hsa-mir-3660; hsa-mir-3661; hsa-mir-3662; hsa-mir-3663; hsa-mir-3664; hsa-mir-3665; hsa-mir-3666; hsa-mir-3667; hsa-mir-3668; hsa-mir-3669; hsa-mir-367; hsa-mir-3670; hsa-mir-3670; hsa-mir-3670; hsa-mir-3671; hsa-mir-3672; hsa-mir-3673; hsa-mir-3674; hsa-mir-3675; hsa-mir-3676; hsa-mir-3677; hsa-mir-3678; hsa-mir-3679; hsa-mir-3680; hsa-mir-3680; hsa-mir-3681; hsa-mir-3682; hsa-mir-3683; hsa-mir-3684; hsa-mir-3685; hsa-mir-3686; hsa-mir-3687; hsa-mir-3688; hsa-mir-3689a; hsa-mir-3689b; hsa-mir-369; hsa-mir-3690; hsa-mir-3691; hsa-mir-3692; hsa-mir-370; hsa-mir-371; hsa-mir-3713; hsa-mir-3714; hsa-mir-372; hsa-mir-373; hsa-mir-374a; hsa-mir-374b; hsa-mir-374c; hsa-mir-375; hsa-mir-376a-1; hsa-mir-376a-2; hsa-mir-376b; hsa-mir-376c; hsa-mir-377; hsa-mir-378; hsa-mir-378b; hsa-mir-378c; hsa-mir-379; hsa-mir-380; hsa-mir-381; hsa-mir-382; hsa-mir-383; hsa-mir-384; hsa-mir-3907; hsa-mir-3908; hsa-mir-3909; hsa-mir-3910-1; hsa-mir-3910-2; hsa-mir-3911; hsa-mir-3912; hsa-mir-3913-1; hsa-mir-3913-2; hsa-mir-3914-1; hsa-mir-3914-2; hsa-mir-3915; hsa-mir-3916; hsa-mir-3917; hsa-mir-3918; hsa-mir-3919; hsa-mir-3920; hsa-mir-3921; hsa-mir-3922; hsa-mir-3923; hsa-mir-3924; hsa-mir-3925; hsa-mir-3926-1; hsa-mir-3926-2; hsa-mir-3927; hsa-mir-3928; hsa-mir-3929; hsa-mir-3934; hsa-mir-3935; hsa-mir-3936; hsa-mir-3937; hsa-mir-3938; hsa-mir-3939; hsa-mir-3940; hsa-mir-3941; hsa-mir-3942; hsa-mir-3943; hsa-mir-3944; hsa-mir-3945; hsa-mir-409; hsa-mir-410; hsa-mir-411; hsa-mir-412; hsa-mir-421; hsa-mir-422a; hsa-mir-423; hsa-mir-424; hsa-mir-425; hsa-mir-4251; hsa-mir-4252; hsa-mir-4253; hsa-mir-4254; hsa-mir-4255; hsa-mir-4256; hsa-mir-4257; hsa-mir-4258; hsa-mir-4259; hsa-mir-4260; hsa-mir-4261; hsa-mir-4262; hsa-mir-4263; hsa-mir-4264; hsa-mir-4265; hsa-mir-4266; hsa-mir-4267; hsa-mir-4268; hsa-mir-4269; hsa-mir-4270; hsa-mir-4271; hsa-mir-4272; hsa-mir-4273; hsa-mir-4274; hsa-mir-4275; hsa-mir-4276; hsa-mir-4277; hsa-mir-4278; hsa-mir-4279; hsa-mir-4280; hsa-mir-4281; hsa-mir-4282; hsa-mir-4283-1; hsa-mir-4283-2; hsa-mir-4284; hsa-mir-4285; hsa-mir-4286; hsa-mir-4287; hsa-mir-4288; hsa-mir-4289; hsa-mir-429; hsa-mir-4290; hsa-mir-4291; hsa-mir-4292; hsa-mir-4293; hsa-mir-4294; hsa-mir-4295; hsa-mir-4296; hsa-mir-4297; hsa-mir-4298; hsa-mir-4299; hsa-mir-4300; hsa-mir-4301; hsa-mir-4302; hsa-mir-4303; hsa-mir-4304; hsa-mir-4305; hsa-mir-4306; hsa-mir-4307; hsa-mir-4308; hsa-mir-4309; hsa-mir-431; hsa-mir-4310; hsa-mir-4311; hsa-mir-4312; hsa-mir-4313; hsa-mir-4314; hsa-mir-4315-1; hsa-mir-4315-2; hsa-mir-4316; hsa-mir-4317; hsa-mir-4318; hsa-mir-4319; hsa-mir-432; hsa-mir-4320; hsa-mir-4321; hsa-mir-4322; hsa-mir-4323; hsa-mir-4324; hsa-mir-4325; hsa-mir-4326; hsa-mir-4327; hsa-mir-4328; hsa-mir-4329; hsa-mir-433; hsa-mir-4330; hsa-mir-448; hsa-mir-449a; hsa-mir-449b; hsa-mir-449c; hsa-mir-450a-1; hsa-mir-450a-2; hsa-mir-450b; hsa-mir-451; hsa-mir-452; hsa-mir-454; hsa-mir-455; hsa-mir-466; hsa-mir-483; hsa-mir-484; hsa-mir-485; hsa-mir-486; hsa-mir-487a; hsa-mir-487b; hsa-mir-488; hsa-mir-489; hsa-mir-490; hsa-mir-491; hsa-mir-492; hsa-mir-493; hsa-mir-494; hsa-mir-495; hsa-mir-496; hsa-mir-497; hsa-mir-498; hsa-mir-499; hsa-mir-500a; hsa-mir-500b; hsa-mir-501; hsa-mir-502; hsa-mir-503; hsa-mir-504; hsa-mir-505; hsa-mir-506; hsa-mir-507; hsa-mir-508; hsa-mir-509-1; hsa-mir-509-2; hsa-mir-509-3; hsa-mir-510; hsa-mir-511-1; hsa-mir-511-2; hsa-mir-512-1; hsa-mir-512-2; hsa-mir-513a-1; hsa-mir-513a-2; hsa-mir-513b; hsa-mir-513c; hsa-mir-514-1; hsa-mir-514-2; hsa-mir-514-3; hsa-mir-514b; hsa-mir-515-1; hsa-mir-515-2; hsa-mir-516a-1; hsa-mir-516a-2; hsa-mir-516b-1; hsa-mir-516b-2; hsa-mir-517a; hsa-mir-517b; hsa-mir-517c; hsa-mir-518a-1; hsa-mir-518a-2; hsa-mir-518b; hsa-mir-518c; hsa-mir-518d; hsa-mir-518e; hsa-mir-518f; hsa-mir-519a-1; hsa-mir-519a-2; hsa-mir-519b; hsa-mir-519c; hsa-mir-519d; hsa-mir-519e; hsa-mir-520a; hsa-mir-520b; hsa-mir-520c; hsa-mir-520d; hsa-mir-520e; hsa-mir-520f; hsa-mir-520g; hsa-mir-520h; hsa-mir-521-1; hsa-mir-521-2; hsa-mir-522; hsa-mir-523; hsa-mir-524; hsa-mir-525; hsa-mir-526a-1; hsa-mir-526a-2; hsa-mir-526b; hsa-mir-527; hsa-mir-532; hsa-mir-539; hsa-mir-541; hsa-mir-542; hsa-mir-543; hsa-mir-544; hsa-mir-544b; hsa-mir-545; hsa-mir-548a-1; hsa-mir-548a-2; hsa-mir-548a-3; hsa-mir-548aa-1; hsa-mir-548aa-2; hsa-mir-548b; hsa-mir-548c; hsa-mir-548d-1; hsa-mir-548d-2; hsa-mir-548e; hsa-mir-548f-1; hsa-mir-548f-2; hsa-mir-548f-3; hsa-mir-548f-4; hsa-mir-548f-5; hsa-mir-548g; hsa-mir-548h-1; hsa-mir-548h-2; hsa-mir-548h-3; hsa-mir-548h-4; hsa-mir-548i-1; hsa-mir-548i-2; hsa-mir-548i-3; hsa-mir-548i-4; hsa-mir-548j; hsa-mir-548k; hsa-mir-5481; hsa-mir-548m; hsa-mir-548n; hsa-mir-548o; hsa-mir-548p; hsa-mir-548q; hsa-mir-548s; hsa-mir-548t; hsa-mir-548u; hsa-mir-548v; hsa-mir-548w; hsa-mir-548x; hsa-mir-548y; hsa-mir-548z; hsa-mir-549; hsa-mir-550a-1; hsa-mir-550a-2; hsa-mir-550b-1; hsa-mir-550b-2; hsa-mir-551a; hsa-mir-551 b; hsa-mir-552; hsa-mir-553; hsa-mir-554; hsa-mir-555; hsa-mir-556; hsa-mir-557; hsa-mir-558; hsa-mir-559; hsa-mir-561; hsa-mir-562; hsa-mir-563; hsa-mir-564; hsa-mir-566; hsa-mir-567; hsa-mir-568; hsa-mir-569; hsa-mir-570; hsa-mir-571; hsa-mir-572; hsa-mir-573; hsa-mir-574; hsa-mir-575; hsa-mir-576; hsa-mir-577; hsa-mir-578; hsa-mir-579; hsa-mir-580; hsa-mir-581; hsa-mir-582; hsa-mir-583; hsa-mir-584; hsa-mir-585; hsa-mir-586; hsa-mir-587; hsa-mir-588; hsa-mir-589; hsa-mir-590; hsa-mir-591; hsa-mir-592; hsa-mir-593; hsa-mir-595; hsa-mir-596; hsa-mir-597; hsa-mir-598; hsa-mir-599; hsa-mir-600; hsa-mir-601; hsa-mir-602; hsa-mir-603; hsa-mir-604; hsa-mir-605; hsa-mir-606; hsa-mir-607; hsa-mir-608; hsa-mir-609; hsa-mir-610; hsa-mir-611; hsa-mir-612; hsa-mir-613; hsa-mir-614; hsa-mir-615; hsa-mir-616; hsa-mir-617; hsa-mir-618; hsa-mir-619; hsa-mir-620; hsa-mir-621; hsa-mir-622; hsa-mir-623; hsa-mir-624; hsa-mir-625; hsa-mir-626; hsa-mir-627; hsa-mir-628; hsa-mir-629; hsa-mir-630; hsa-mir-631; hsa-mir-632; hsa-mir-633; hsa-mir-634; hsa-mir-635; hsa-mir-636; hsa-mir-637; hsa-mir-638; hsa-mir-639; hsa-mir-640; hsa-mir-641; hsa-mir-642a; hsa-mir-642b; hsa-mir-643; hsa-mir-644; hsa-mir-645; hsa-mir-646; hsa-mir-647; hsa-mir-648; hsa-mir-649; hsa-mir-650; hsa-mir-651; hsa-mir-652; hsa-mir-653; hsa-mir-654; hsa-mir-655; hsa-mir-656; hsa-mir-657; hsa-mir-658; hsa-mir-659; hsa-mir-660; hsa-mir-661; hsa-mir-662; hsa-mir-663; hsa-mir-663b; hsa-mir-664; hsa-mir-665; hsa-mir-668; hsa-mir-670; hsa-mir-671; hsa-mir-675; hsa-mir-676; hsa-mir-708; hsa-mir-7-1; hsa-mir-711; hsa-mir-718; hsa-mir-7-2; hsa-mir-720; hsa-mir-7-3; hsa-mir-744; hsa-mir-758; hsa-mir-759; hsa-mir-760; hsa-mir-761; hsa-mir-762; hsa-mir-764; hsa-mir-765; hsa-mir-766; hsa-mir-767; hsa-mir-769; hsa-mir-770; hsa-mir-802; hsa-mir-873; hsa-mir-874; hsa-mir-875; hsa-mir-876; hsa-mir-877; hsa-mir-885; hsa-mir-887; hsa-mir-888; hsa-mir-889; hsa-mir-890; hsa-mir-891a; hsa-mir-891b; hsa-mir-892a; hsa-mir-892b; hsa-mir-9-1; hsa-mir-9-2; hsa-mir-920; hsa-mir-921; hsa-mir-922; hsa-mir-924; hsa-mir-92a-1; hsa-mir-92a-2; hsa-mir-92b; hsa-mir-93; hsa-mir-9-3; hsa-mir-933; hsa-mir-934; hsa-mir-935; hsa-mir-936; hsa-mir-937; hsa-mir-938; hsa-mir-939; hsa-mir-940; hsa-mir-941-1; hsa-mir-941-3; hsa-mir-941-4; hsa-mir-942; hsa-mir-943; hsa-mir-944; hsa-mir-95; hsa-mir-96; hsa-mir-98; hsa-mir-99a; hsa-mir-99b.ebv-miR-BHRFI-1, ebv-miR-BHRFI-2*, ebv-miR-BHRFI-2, ebv-miR-BHRFI-3, ebv-miR-BARTI-5p, ebv-miR-BART2, hcmv-miR-UL22A, hcmv-miR-UL22A*, hcmv-miR-UL36, hcmv-miR-UL112, hcmv-miR-UL148D, hcmv-miR-US5-1, hcmv-miR-US5-2, hcmv-miR-US25-I, hcmv-miR-US25-2-5p, hcmv-miR-US25-2-3p, hcmv-miR-US33, kshv-miR-K12-10a, kshv-miR-K12-10b, kshv-miR-K12-11, kshv-miR-K12-I, kshv-miR-K12-2, kshv-miR-K12-9*, kshv-miR-K12-9, kshv-miR-K12-8, kshv-miR-K12-7, kshv-miR-K12-6-5p, kshv-miR-K12-6-3p, kshv-miR-K12-5, kshv-miR-K12-4-5p, kshv-miR-K12-4-3p, kshv-miR-K12-3, kshv-miR-K12-3*, hcmv-miR-US4, hcmv-miR-UL70-5p, hcmv-20 miR-UL70-3p, ebv-miR-BART3-5p, ebv-miR-BART3-3p, ebv-miR-BART4, ebv-miR-BART5, ebv-miR-BART6-5p, ebv-miR-BART6-3p, ebv-miR-BART7, ebv-miR-BART8-5p, ebv-miR-BART8-3p, ebv-miR-BART9, ebv-miR-BARTIO, ebv-miR-BARTII-5p, ebv-miR-BARTII-3p, ebv-miR-BART12, ebv-miR-BART13, ebv-miR-BART14-5p, ebv-miR-BART14-3p, kshv-miR-25 K12-12, ebv-miR-BART15, ebv-miR-BART16, ebv-miR-BART17-5p, ebv-miR-BART17-3p, ebv-miR-BART18, ebv-miR-BART19, ebv-miR-BART20-5p, ebv-miR-BART20-3p, and hsvl-miR-HI.

### BRIEF DESCRIPTION OF THE SEVERAL VIEWS OF THE DRAWINGS

FIG. 1 shows a representative whole-body autoradiogram of the radioactivity distribution in a male CD-1 mouse at 0.167 hours after IV administration of 20 mg/kg ¹⁴C labeled MI-01452.

FIG. 2 shows a representative whole-body autoradiogram of the radioactivity distribution in a male CD-1 mouse at 0.5 hours after IV administration of 20 mg/kg ¹⁴C labeled MI-01452.

FIG. 3 shows a representative whole-body autoradiogram of the radioactivity distribution in a male CD-1 mouse at 1 hour after IV administration of 20 mg/kg ¹⁴C labeled MI-01452.

FIG. 4 shows a representative whole-body autoradiogram of the radioactivity distribution in a male CD-1 mouse at 2 hours after IV administration of 20 mg/kg ¹⁴C labeled MI-01452.

FIG. 5 shows a representative whole-body autoradiogram of the radioactivity distribution in a male CD-1 mouse at 4 hours after IV administration of 20 mg/kg ¹⁴C labeled MI-01452.

FIG. 6 shows a representative whole-body autoradiogram of the radioactivity distribution in a male CD-1 mouse at 8 hours after IV administration of 20 mg/kg ¹⁴C labeled MI-01452.

FIG. 7 shows a representative whole-body autoradiogram of the radioactivity distribution in a male CD-1 mouse at 24 hours after IV administration of 20 mg/kg ¹⁴C labeled MI-01452.

FIG. 8 shows a representative whole-body autoradiogram of the radioactivity distribution in a male CD-1 mouse at 0.167 hours after IV administration of 20 mg/kg ¹⁴C labeled MI-01453.

FIG. 9 shows a representative whole-body autoradiogram of the radioactivity distribution in a male CD-1 mouse at 0.5 hours after IV administration of 20 mg/kg ¹⁴C labeled MI-01453.

FIG. 10 shows a representative whole-body autoradiogram of the radioactivity distribution in a male CD-1 mouse at 1 hour after IV administration of 20 mg/kg ¹⁴C labeled MI-01453.

FIG. 11 shows a representative whole-body autoradiogram of the radioactivity distribution in a male CD-1 mouse at 2 hours after IV administration of 20 mg/kg ¹⁴C labeled MI-01453.

FIG. 12 shows a representative whole-body autoradiogram of the radioactivity distribution in a male CD-1 mouse at 4 hours after IV administration of 20 mg/kg ¹⁴C labeled MI-01453.

FIG. 13 shows a representative whole-body autoradiogram of the radioactivity distribution in a male CD-1 mouse at 8 hours after IV administration of 20 mg/kg ¹⁴C labeled MI-01453.

FIG. 14 shows a representative whole-body autoradiogram of the radioactivity distribution in a male CD-1 mouse at 24 hours after IV administration of 20 mg/kg ¹⁴C labeled MI-01453.

FIG. 15 shows a representative whole-body autoradiogram of the radioactivity distribution in a male CD-1 mouse at 0.167 hours after IV administration of 20 mg/kg ¹⁴C labeled MI-01454.

FIG. 16 shows a representative whole-body autoradiogram of the radioactivity distribution in a male CD-1 mouse at 0.5 hours after IV administration of 20 mg/kg ¹⁴C labeled MI-01454.

FIG. 17 shows a representative whole-body autoradiogram of the radioactivity distribution in a male CD-1 mouse at 1 hour after IV administration of 20 mg/kg ¹⁴C labeled MI-01454.

FIG. 18 shows a representative whole-body autoradiogram of the radioactivity distribution in a male CD-1 mouse at 2 hours after IV administration of 20 mg/kg ¹⁴C labeled MI-01454.

FIG. 19 shows a representative whole-body autoradiogram of the radioactivity distribution in a male CD-1 mouse at 4 hours after IV administration of 20 mg/kg ¹⁴C labeled MI-01454.

FIG. 20 shows a representative whole-body autoradiogram of the radioactivity distribution in a male CD-1 mouse at 8 hours after IV administration of 20 mg/kg ¹⁴C labeled MI-01454.

FIG. 21 shows a representative whole-body autoradiogram of the radioactivity distribution in a male CD-1 mouse at 24 hours after IV administration of 20 mg/kg ¹⁴C labeled MI-01454.

FIG. 22 shows the distribution of anti-miRNA concentration in tissues of the reproductive, muscular, and respiratory tract over time. Each group of mice received IV administration of 20 mg/kg ¹⁴C labeled anti-miRNAs. Group I mice were administered [¹⁴C] MI-01452 (FIG. 22A); group II mice were administered [¹⁴C] MI-01453 (FIG. 22B); and group III mice were administered [¹⁴C] MI-01454 (FIG. 22C).

FIG. 23 shows the distribution of anti-miRNA concentration in tissues of the central nervous system (CNS), endocrine and secretory system over time. Each group of mice received IV administration of 20 mg/kg ¹⁴C labeled anti-miRNAs. Group I mice were administered [¹⁴C] MI-01452 (FIG. 23A); group II mice were administered [¹⁴C] MI-01453 (FIG. 23B); and group III mice were administered [¹⁴C] MI-01454 (FIG. 23C).

FIG. 24 shows the distribution of anti-miRNA concentration in tissues of the vascular and excretory system over time. Each group of mice received IV administration of 20 mg/kg ¹⁴C labeled anti-miRNAs. Group I mice were administered [¹⁴C] MI-01452 (FIG. 24A); group II mice were administered [¹⁴C] MI-01453 (FIG. 24B); and group III mice were administered [¹⁴C] MI-01454 (FIG. 24C).

FIG. 25 shows a data table of concentrations (μg equiv/g) of radioactivity in tissues of male CD 1 mice after a single IV dose of [¹⁴C] MI-01452 at 20 mg/kg (325 μCi/kg).

FIG. 26 shows a data table of concentrations (μg equiv/g) of radioactivity in tissues of male CD 1 mice after a single IV dose of [¹⁴C] MI-01453 at 20 mg/kg (325 μCi/kg).

FIG. 27 shows a data table of concentrations (μg equiv/g) of radioactivity in tissues of male CD 1 mice after a single IV dose of [¹⁴C] MI-01454 at 20 mg/kg (260 μCi/kg).

### BRIEF DESCRIPTION OF THE SEQUENCE IDENTIFIERS

SEQ ID NO: 1 sets forth the nucleic acid sequence of an anti-miRNA.

SEQ ID NO: 2 sets forth the nucleic acid sequence of an anti-miRNA.

SEQ ID NO: 3 sets forth the nucleic acid sequence of an anti-miRNA.

### DETAILED DESCRIPTION

### A. Overview

Broad-based delivery of anti-miRNA molecules, siRNAs, shRNAs, piRNA mimetics, miRNA mimetics and other nucleic acid-based therapeutics to tissues is a major hurdle to the clinical use of these molecules. The limited successes of oligomer-based therapeutic applications use local administration in easily accessible tissues such as eyes (Shen et al., Suppression of ocular neovascularization with siRNA targeting VEGF receptor 1. Gene Ther (2006). 13: 225-234), lungs (Bitko et al., Inhibition of respiratory viruses by nasally administered siRNA. Nat Med (2005) 11: 50-55) and vagina (Palliser et al. An siRNA-based microbicide protects mice from lethal herpes simplex virus 2 infection. Nature (2006) 439: 89-94). Systemic administration of RNA-based therapeutics results in poor biodistribution: the therapeutics preferentially accumulate in the kidney and liver (Shayne C. Gad. Drug Discovery Handbook. (2005). John Wiley & Sons. P. 1281).

Thus, while anti-miRNA molecules, siRNAs, shRNAs, piRNA mimetics, miRNA mimetics are attractive drug candidates, little progress has been made in overcoming the poor biodistribution problems associated with systemic administration of these candidate drugs. Present strategies for systemic administration of such compounds are ineffective in delivering them to a broad range of tissues. Without developing more effective reagents to facilitate tissue uptake, these compounds will continue to poor clinical options to treat disease. Accordingly, there is an important need for oligomers with broad biodistribution profiles and that efficiently regulate gene expression and miRNA expression levels.

The present invention provides much needed solutions for the treatment of diseases, disorders, and conditions associated with increased gene expression or miRNA expression. The present invention provides novel oligomers that have substantial advantages over prior art methods of targeting genes and miRNAs. The inventive oligomers have surprisingly and unexpectedly broad biodistribution profiles and provide a safer and provide a more robust treatment paradigm associated with less off-target effects. In various embodiments, the present invention contemplates, in part, methods of providing a broadly biodistributed oligomer conjugated to at least one MGB moiety. Thus, administration of the inventive oligomers and compositions comprising the same, provide a novel and highly desirable strategy for delivering them to wide variety of tissues within a subject. Accordingly, the methods and compositions of the present invention offer preferred strategies for regulating gene expression and expression of miRNAs and miRNA clusters in a wide variety of cells and tissues.

### B. siRNAs and RNAi

RNA interference refers to the process of sequence-specific post-transcriptional gene silencing in animals mediated by short interfering RNAs (siRNAs) (Zamore et al., 2000, Cell, 101, 25-33; Fire et al., 1998, Nature, 391, 806; Hamilton et al., 1999, Science, 286, 950-951; Lin et al., 1999, Nature, 402, 128-129; Sharp, 1999, Genes & Dev., 13, 139-141; and Strauss, 1999, Science, 286, 886).

The presence of long dsRNAs in cells stimulates the activity of a ribonuclease III enzyme referred to as Dicer (Bass, 2000, Cell, 101, 235; Zamore et al., 2000, Cell, 101, 25-33; Hammond et al., 2000, Nature, 404, 293). Dicer is involved in the processing of the dsRNA into short pieces of dsRNA known as short interfering RNAs (siRNAs) (Zamore et al., 2000, Cell, 101, 25-33; Bass, 2000, Cell, 101, 235; Berstein et al., 2001, Nature, 409, 363). Short interfering RNAs derived from dicer activity are typically about 21 to about 23 nucleotides in length and comprise about 19 base pair duplexes (Zamore et al., 2000, Cell, 101, 25-33; Elbashir et al., 2001, Genes Dev., 15, 188). Dicer has also been implicated in the excision of 21- and 22-nucleotide small temporal RNAs (stRNAs) from precursor RNA of conserved structure that are implicated in translational control (Hutvagner et al., 2001, Science, 293, 834). The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex (Elbashir et al., 2001, Genes Dev., 15, 188).

Elbashir et al., 2001, Nature, 411, 494 and Tuschl et al., International PCT Publication No. WO 01/75164, describe RNAi induced by introduction of duplexes of synthetic 21-nucleotide RNAs in cultured mammalian cells including human embryonic kidney and HeLa cells. Recent work in Drosophila embryonic lysates (Elbashir et al., 2001, EMBO J., 20, 6877 and Tuschl et al., International PCT Publication No. WO 01/75164) has revealed certain requirements for siRNA length, structure, chemical composition, and sequence that are essential to mediate efficient RNAi activity. These studies have shown that 21-nucleotide siRNA duplexes are most active when containing 3′-terminal dinucleotide overhangs.

Furthermore, complete substitution of one or both siRNA strands with 2′-deoxy (2′-H) or 2′-O-methyl nucleotides abolishes RNAi activity, whereas substitution of the 3′-terminal siRNA overhang nucleotides with 2′-deoxy nucleotides (2′-H) was shown to be tolerated. Single mismatch sequences in the center of the siRNA duplex were also shown to abolish RNAi activity. In addition, these studies also indicate that the position of the cleavage site in the target RNA is defined by the 5′-end of the siRNA guide sequence rather than the 3′-end of the guide sequence (Elbashir et al., 2001, EMBO J, 20, 6877). Other studies have indicated that a 5′-phosphate on the target-complementary strand of a siRNA duplex is required for siRNA activity and that ATP is utilized to maintain the 5′-phosphate moiety on the siRNA (Nykanen et al., 2001, Cell, 107, 309).

The use of longer dsRNA has been described. For example, Tuschl et al., International PCT Publication No. WO 01/75164, describe a Drosophila in vitro RNAi system and the use of specific siRNA molecules for certain functional genomic and certain therapeutic applications. Fire et al., International PCT Publication No. WO 99/32619, describe particular methods for introducing certain long dsRNA molecules into cells for use in inhibiting gene expression in nematodes. Mello et al., International PCT Publication No. WO 01/29058, describe the identification of specific genes involved in dsRNA-mediated RNAi. Driscoll et al., International PCT Publication No. WO 01/49844, describe specific DNA expression constructs for use in facilitating gene silencing in targeted organisms. Fire et al., U.S. Pat. No. 6,506,559, describe certain methods for inhibiting gene expression in vitro using certain long dsRNA (299 bp-1033 bp) constructs that mediate RNAi.

Illustrative mechanisms of RNA interference include, but are not limited to post transcriptional gene silencing, translational inhibition, transcriptional inhibition, or epigenetic RNAi. For example, siRNA molecules of the invention can be used to epigenetically silence genes at both the post-transcriptional level or the pre-transcriptional level. In a non-limiting example, epigenetic modulation of gene expression by siRNA molecules of the invention can result from siRNA mediated modification of chromatin structure or methylation patterns to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237). In another non-limiting example, modulation of gene expression by siRNA molecules of the invention can result from siRNA mediated cleavage of RNA (either coding or non-coding RNA) via RISC, or alternately, translational inhibition as is known in the art. In a further non-limiting example embodiment, modulation of gene expression by siRNA molecules of the invention can result from transcriptional inhibition (see for example Janowski et al., 2005, Nature Chemical Biology, 1, 216-222).

The present invention further contemplates, in part, oligomers that are referred to as “siRNAs” or “short interfering RNAs”. As used herein the terms “siRNA” and “short interfering RNA” refer to unimolecular nucleic acids and to nucleic acids comprised of two separate strands that are capable of performing RNAi and that have a duplex region that is between 14 and 30 base pairs in length. Additionally, the term sRNA and the phrase “short interfering RNA” include nucleic acids that also contain moieties other than ribonucleotide moieties, including, but not limited to, modified nucleotides, modified internucleotide linkages, non-nucleotides, deoxynucleotides and analogs of the aforementioned nucleotides. siRNAs can be duplexes, and can also comprise short hairpin RNAs (shRNAs), RNAs with loops as long as, for example, 4 to 23 or more nucleotides, RNAs with stem loop bulges, microRNAs, and short temporal RNAs. RNAs having loops or hairpin loops can include structures where the loops are connected to the stem by linkers such as flexible linkers. Flexible linkers can be comprised of a wide variety of chemical structures, as long as they are of sufficient length and materials to enable effective intramolecular hybridization of the stem elements. Typically, the length to be spanned is at least about 10-24 atoms. When the siRNAs are hairpins, the sense strand and antisense strand are part of one longer molecule.

### C. miRNAs

microRNAs (miRNAs) are important in biological processes that underlie disease, developmental timing, differentiation, apoptosis, cell proliferation, organ development, and metabolism. The biogenesis of miRNAs is well-known and has been reviewed in Kim, MicroRNA biogenesis: coordinated cropping and dicing. Nature Reviews Molecular Cell Biology, 6 (May 2005): 376-385 and in He and Hannon, MicroRNAs: Small RNAs with a big role in gene regulation. Nature Reviews Genetics, 5 (2004): 522-531.

Most miRNA genes are transcribed by RNA polymerase II (Pol II). A transcribed miRNA transcript is known as a primary miRNA (pri-miRNA or pri-miRNA transcript). The majority of known Pol II transcribed miRNAs are found in intergenic regions or in anti-sense orientation to genes and contain their own miRNA gene promoter and regulatory units. Such pri-miRNAs can be hundreds or thousands of nucleotides in length and are spliced, capped at the 5′ end, and polyadenylated, e.g., a poly(A) tail. Many miRNAs are encoded in clusters of two of more miRNA genes; thus, pri-miRNAs transcribed from an miRNA gene cluster under the control of an miRNA gene promoter are known as polycistronic pri-miRNA transcripts.

Another significant portion of known miRNAs transcribed Pol II are encoded within existing genes, e.g., in 5′ or 3′ untranslated regions (UTRs), within introns, and non-protein coding genes. Usually, such miRNAs are in the sense orientation and are transcribed as part of the host gene pre-mRNA and/or mRNA transcript. Thus, in this instance, the pre-mRNA (e.g., intronic miRNA) or mRNA transcript of the host gene comprises the pri-miRNA transcript. Similarly, when the miRNA genes are clustered in the host gene, the pre-mRNA (e.g., intronic miRNA) or mRNA transcript of the host gene comprises the polycistronic pri-miRNA transcript of the miRNA gene cluster.

The pri-miRNA transcripts are cleaved in the nucleus by the nuclear RNase III Drosha to release the precursor of miRNA, smaller stem-loop, hairpin structure of ˜65-70 nt in length (pre-miRNA). Following nuclear processing by Drosha, pre-miRNAs are exported by Exportin-5 to the cytoplasm. Once there, the RNase III enzyme Dicer processes the pre-miRNAs into ˜18-24 nt duplexes. One strand of the duplex, which incorporates into RNA-induced silencing complex (RISC) and silences the gene expression is called the guide strand, or miRNA; while the other strand of duplex is degraded and called the passenger strand, or miRNA* (star strand).

Without wishing to be bound by any particular theory, it is believed that depending on the thermodynamic stability of the 5′-strand and the 3′-strand in the stem-loop structure of a pre-miRNA, cells preferentially select the less stable one to be the guide strand and destroy the miRNA* or passenger strand. However, target prediction and validation assays demonstrated that both strands of a miRNA pair could target equal numbers of genes and that both were able to suppress the expression of their target genes. Using a dataset of 347 miRNA precursors (pre-miRNAs), Hu and colleagues determined that more than 73% of the precursors generated mature miRNAs from both the guide strand and passenger strand (Hu et al., Sequence features associated with microRNA strand selection in humans and flies. BMC Genomics, 10 (2009): 413). Accordingly, biologically active miRNAs can be produced from either strand of the ˜18-24 nt duplex.

Thus, as used herein, the term “miRNA” or the abbreviation “miR-” followed by a number refers to the biologically active single stranded RNA that is incorporated into a silencing complex and has RNA silencing activity (miRNA activity). When particular miRNAs are discussed, the naming conventions used are the same used at the miRBase microRNA database (www.mirbase.org). For example, if miRNA cloning studies have identify two ˜18-24 nt sequences miRNAs which originate from the same predicted precursor and the relative abundancies indicate which is the predominantly expressed miRNA, the mature sequences are assigned names of the form miR-17 (the predominant product; the guide strand) and miR-17* (from the opposite arm of the precursor; the passenger or strand). When the data are not sufficient to determine which sequence is the predominant one, names like miR-142-5p (from the 5′ arm) and miR-142-3p (from the 3′ arm) are used. In cases where the data are not sufficient to determine which sequence is the predominant one, the 5′ arm is named the guide strand sequence and the 3′ arm is named the passenger strand sequence as a means to use common terminology to refer to the sequences.

The number of human miRNAs reported so far (the September 2010 release of miRBase at the Sanger Institute) is 1049, nearly five times as many as initial calculations indicated. Additionally, many more predicted miRNA genes are awaiting experimental confirmation.

Illustrative target miRNAs include, but are not limited to: hsa-let-7a-1; hsa-let-7a-2; hsa-let-7a-3; hsa-let-7b; hsa-let-7c; hsa-let-7d; hsa-let-7e; hsa-let-7f-1; hsa-let-7f-2; hsa-let-7g; hsa-let-7i; hsa-mir-100; hsa-mir-101-1; hsa-mir-101-2; hsa-mir-103-1; hsa-mir-103-1-as; hsa-mir-103-2; hsa-mir-103-2-as; hsa-mir-105-1; hsa-mir-105-2; hsa-mir-106a; hsa-mir-106b; hsa-mir-107; hsa-mir-10a; hsa-mir-10b; hsa-mir-1-1; hsa-mir-1178; hsa-mir-1179; hsa-mir-1180; hsa-mir-1181; hsa-mir-1182; hsa-mir-1183; hsa-mir-1184-1; hsa-mir-1184-2; hsa-mir-1184-3; hsa-mir-1185-1; hsa-mir-1185-2; hsa-mir-1193; hsa-mir-1197; hsa-mir-1-2; hsa-mir-1200; hsa-mir-1202; hsa-mir-1203; hsa-mir-1204; hsa-mir-1205; hsa-mir-1206; hsa-mir-1207; hsa-mir-1208; hsa-mir-122; hsa-mir-1224; hsa-mir-1225; hsa-mir-1226; hsa-mir-1227; hsa-mir-1228; hsa-mir-1229; hsa-mir-1231; hsa-mir-1233-1; hsa-mir-1233-2; hsa-mir-1234; hsa-mir-1236; hsa-mir-1237; hsa-mir-1238; hsa-mir-124-1; hsa-mir-124-2; hsa-mir-1243; hsa-mir-124-3; hsa-mir-1244-1; hsa-mir-1244-2; hsa-mir-1244-3; hsa-mir-1245; hsa-mir-1246; hsa-mir-1247; hsa-mir-1248; hsa-mir-1249; hsa-mir-1250; hsa-mir-1251; hsa-mir-1252; hsa-mir-1253; hsa-mir-1254; hsa-mir-1255a; hsa-mir-1255b-1; hsa-mir-1255b-2; hsa-mir-1256; hsa-mir-1257; hsa-mir-1258; hsa-mir-125a; hsa-mir-125b-1; hsa-mir-125b-2; hsa-mir-126; hsa-mir-1260; hsa-mir-1260b; hsa-mir-1261; hsa-mir-1262; hsa-mir-1263; hsa-mir-1264; hsa-mir-1265; hsa-mir-1266; hsa-mir-1267; hsa-mir-1268; hsa-mir-1269; hsa-mir-127; hsa-mir-1270-1; hsa-mir-1270-2; hsa-mir-1271; hsa-mir-1272; hsa-mir-1273; hsa-mir-1273c; hsa-mir-1273d; hsa-mir-1274a; hsa-mir-1274b; hsa-mir-1275; hsa-mir-1276; hsa-mir-1277; hsa-mir-1278; hsa-mir-1279; hsa-mir-1280; hsa-mir-1281; hsa-mir-128-1; hsa-mir-1282; hsa-mir-128-2; hsa-mir-1283-1; hsa-mir-1283-2; hsa-mir-1284; hsa-mir-1285-1; hsa-mir-1285-2; hsa-mir-1286; hsa-mir-1287; hsa-mir-1288; hsa-mir-1289-1; hsa-mir-1289-2; hsa-mir-1290; hsa-mir-1291; hsa-mir-129-1; hsa-mir-1292; hsa-mir-129-2; hsa-mir-1293; hsa-mir-1294; hsa-mir-1295; hsa-mir-1296; hsa-mir-1297; hsa-mir-1298; hsa-mir-1299; hsa-mir-1301; hsa-mir-1302-1; hsa-mir-1302-10; hsa-mir-1302-11; hsa-mir-1302-2; hsa-mir-1302-3; hsa-mir-1302-4; hsa-mir-1302-5; hsa-mir-1302-6; hsa-mir-1302-7; hsa-mir-1302-8; hsa-mir-1302-9; hsa-mir-1303; hsa-mir-1304; hsa-mir-1305; hsa-mir-1306; hsa-mir-1307; hsa-mir-130a; hsa-mir-130b; hsa-mir-132; hsa-mir-1321; hsa-mir-1322; hsa-mir-1323; hsa-mir-1324; hsa-mir-133a-1; hsa-mir-133a-2; hsa-mir-133b; hsa-mir-134; hsa-mir-135a-1; hsa-mir-135a-2; hsa-mir-135b; hsa-mir-136; hsa-mir-137; hsa-mir-138-1; hsa-mir-138-2; hsa-mir-139; hsa-mir-140; hsa-mir-141; hsa-mir-142; hsa-mir-143; hsa-mir-144; hsa-mir-145; hsa-mir-1468; hsa-mir-1469; hsa-mir-146a; hsa-mir-146b; hsa-mir-147; hsa-mir-1470; hsa-mir-1471; hsa-mir-147b; hsa-mir-148a; hsa-mir-148b; hsa-mir-149; hsa-mir-150; hsa-mir-151; hsa-mir-152; hsa-mir-153-1; hsa-mir-153-2; hsa-mir-1537; hsa-mir-1538; hsa-mir-1539; hsa-mir-154; hsa-mir-155; hsa-mir-15a; hsa-mir-15b; hsa-mir-16-1; hsa-mir-16-2; hsa-mir-17; hsa-mir-181a-1; hsa-mir-181a-2; hsa-mir-181 b-1; hsa-mir-181 b-2; hsa-mir-181c; hsa-mir-181d; hsa-mir-182; hsa-mir-1825; hsa-mir-1827; hsa-mir-183; hsa-mir-184; hsa-mir-185; hsa-mir-186; hsa-mir-187; hsa-mir-188; hsa-mir-18a; hsa-mir-18b; hsa-mir-190; hsa-mir-1908; hsa-mir-1909; hsa-mir-190b; hsa-mir-191; hsa-mir-1910; hsa-mir-1911; hsa-mir-1912; hsa-mir-1913; hsa-mir-1914; hsa-mir-1915; hsa-mir-192; hsa-mir-193a; hsa-mir-193b; hsa-mir-194-1; hsa-mir-194-2; hsa-mir-195; hsa-mir-196a-1; hsa-mir-196a-2; hsa-mir-196b; hsa-mir-197; hsa-mir-1972-1; hsa-mir-1972-2; hsa-mir-1973; hsa-mir-1976; hsa-mir-198; hsa-mir-199a-1; hsa-mir-199a-2; hsa-mir-199b; hsa-mir-19a; hsa-mir-19b-1; hsa-mir-19b-2; hsa-mir-200a; hsa-mir-200b; hsa-mir-200c; hsa-mir-202; hsa-mir-203; hsa-mir-204; hsa-mir-205; hsa-mir-2052; hsa-mir-2053; hsa-mir-2054; hsa-mir-206; hsa-mir-208a; hsa-mir-208b; hsa-mir-20a; hsa-mir-20b; hsa-mir-21; hsa-mir-210; hsa-mir-211; hsa-mir-2110; hsa-mir-2113; hsa-mir-2114; hsa-mir-2115; hsa-mir-2116; hsa-mir-2117; hsa-mir-212; hsa-mir-214; hsa-mir-215; hsa-mir-216a; hsa-mir-216b; hsa-mir-217; hsa-mir-218-1; hsa-mir-218-2; hsa-mir-219-1; hsa-mir-219-2; hsa-mir-22; hsa-mir-221; hsa-mir-222; hsa-mir-223; hsa-mir-224; hsa-mir-2276; hsa-mir-2277; hsa-mir-2278; hsa-mir-2355; hsa-mir-23a; hsa-mir-23b; hsa-mir-23c; hsa-mir-24-1; hsa-mir-24-2; hsa-mir-25; hsa-mir-26a-1; hsa-mir-26a-2; hsa-mir-26b; hsa-mir-27a; hsa-mir-27b; hsa-mir-28; hsa-mir-2861; hsa-mir-2909; hsa-mir-296; hsa-mir-297; hsa-mir-298; hsa-mir-299; hsa-mir-29a; hsa-mir-29b-1; hsa-mir-29b-2; hsa-mir-29c; hsa-mir-300; hsa-mir-301a; hsa-mir-301b; hsa-mir-302a; hsa-mir-302b; hsa-mir-302c; hsa-mir-302d; hsa-mir-302e; hsa-mir-302f; hsa-mir-3065; hsa-mir-3074; hsa-mir-30a; hsa-mir-30b; hsa-mir-30c-1; hsa-mir-30c-2; hsa-mir-30d; hsa-mir-30e; hsa-mir-31; hsa-mir-3115; hsa-mir-3116-1; hsa-mir-3116-2; hsa-mir-3117; hsa-mir-3118-1; hsa-mir-3118-2; hsa-mir-3118-3; hsa-mir-3118-4; hsa-mir-3118-5; hsa-mir-3118-6; hsa-mir-3119-1; hsa-mir-3119-2; hsa-mir-3120; hsa-mir-3121; hsa-mir-3122; hsa-mir-3123; hsa-mir-3124; hsa-mir-3125; hsa-mir-3126; hsa-mir-3127; hsa-mir-3128; hsa-mir-3129; hsa-mir-3130-1; hsa-mir-3130-2; hsa-mir-3131; hsa-mir-3132; hsa-mir-3133; hsa-mir-3134; hsa-mir-3135; hsa-mir-3136; hsa-mir-3137; hsa-mir-3138; hsa-mir-3139; hsa-mir-3140; hsa-mir-3141; hsa-mir-3142; hsa-mir-3143; hsa-mir-3144; hsa-mir-3145; hsa-mir-3146; hsa-mir-3147; hsa-mir-3148; hsa-mir-3149; hsa-mir-3150; hsa-mir-3150b; hsa-mir-3151; hsa-mir-3152; hsa-mir-3153; hsa-mir-3154; hsa-mir-3155; hsa-mir-3156-1; hsa-mir-3156-2; hsa-mir-3156-3; hsa-mir-3157; hsa-mir-3158-1; hsa-mir-3158-2; hsa-mir-3159; hsa-mir-3160-1; hsa-mir-3160-2; hsa-mir-3161; hsa-mir-3162; hsa-mir-3163; hsa-mir-3164; hsa-mir-3165; hsa-mir-3166; hsa-mir-3167; hsa-mir-3168; hsa-mir-3169; hsa-mir-3170; hsa-mir-3171; hsa-mir-3173; hsa-mir-3174; hsa-mir-3175; hsa-mir-3176; hsa-mir-3177; hsa-mir-3178; hsa-mir-3179-1; hsa-mir-3179-2; hsa-mir-3179-3; hsa-mir-3180-1; hsa-mir-3180-2; hsa-mir-3180-3; hsa-mir-3180-4; hsa-mir-3180-5; hsa-mir-3181; hsa-mir-3182; hsa-mir-3183; hsa-mir-3184; hsa-mir-3185; hsa-mir-3186; hsa-mir-3187; hsa-mir-3188; hsa-mir-3189; hsa-mir-3190; hsa-mir-3191; hsa-mir-3192; hsa-mir-3193; hsa-mir-3194; hsa-mir-3195; hsa-mir-3196; hsa-mir-3197; hsa-mir-3198; hsa-mir-3199-1; hsa-mir-3199-2; hsa-mir-32; hsa-mir-3200; hsa-mir-3201; hsa-mir-3202-1; hsa-mir-3202-2; hsa-mir-320a; hsa-mir-320b-1; hsa-mir-320b-2; hsa-mir-320c-1; hsa-mir-320c-2; hsa-mir-320d-1; hsa-mir-320d-2; hsa-mir-320e; hsa-mir-323; hsa-mir-323b; hsa-mir-324; hsa-mir-325; hsa-mir-326; hsa-mir-328; hsa-mir-329-1; hsa-mir-329-2; hsa-mir-330; hsa-mir-331; hsa-mir-335; hsa-mir-337; hsa-mir-338; hsa-mir-339; hsa-mir-33a; hsa-mir-33b; hsa-mir-340; hsa-mir-342; hsa-mir-345; hsa-mir-346; hsa-mir-34a; hsa-mir-34b; hsa-mir-34c; hsa-mir-3605; hsa-mir-3606; hsa-mir-3607; hsa-mir-3609; hsa-mir-361; hsa-mir-3610; hsa-mir-3611; hsa-mir-3612; hsa-mir-3613; hsa-mir-3614; hsa-mir-3615; hsa-mir-3616; hsa-mir-3617; hsa-mir-3618; hsa-mir-3619; hsa-mir-362; hsa-mir-3620; hsa-mir-3621; hsa-mir-3622a; hsa-mir-3622b; hsa-mir-363; hsa-mir-3646; hsa-mir-3647; hsa-mir-3648; hsa-mir-3649; hsa-mir-3650; hsa-mir-3651; hsa-mir-365-1; hsa-mir-3652; hsa-mir-365-2; hsa-mir-3653; hsa-mir-3654; hsa-mir-3655; hsa-mir-3656; hsa-mir-3657; hsa-mir-3658; hsa-mir-3659; hsa-mir-3660; hsa-mir-3661; hsa-mir-3662; hsa-mir-3663; hsa-mir-3664; hsa-mir-3665; hsa-mir-3666; hsa-mir-3667; hsa-mir-3668; hsa-mir-3669; hsa-mir-367; hsa-mir-3670; hsa-mir-3670; hsa-mir-3670; hsa-mir-3671; hsa-mir-3672; hsa-mir-3673; hsa-mir-3674; hsa-mir-3675; hsa-mir-3676; hsa-mir-3677; hsa-mir-3678; hsa-mir-3679; hsa-mir-3680; hsa-mir-3680; hsa-mir-3681; hsa-mir-3682; hsa-mir-3683; hsa-mir-3684; hsa-mir-3685; hsa-mir-3686; hsa-mir-3687; hsa-mir-3688; hsa-mir-3689a; hsa-mir-3689b; hsa-mir-369; hsa-mir-3690; hsa-mir-3691; hsa-mir-3692; hsa-mir-370; hsa-mir-371; hsa-mir-3713; hsa-mir-3714; hsa-mir-372; hsa-mir-373; hsa-mir-374a; hsa-mir-374b; hsa-mir-374c; hsa-mir-375; hsa-mir-376a-1; hsa-mir-376a-2; hsa-mir-376b; hsa-mir-376c; hsa-mir-377; hsa-mir-378; hsa-mir-378b; hsa-mir-378c; hsa-mir-379; hsa-mir-380; hsa-mir-381; hsa-mir-382; hsa-mir-383; hsa-mir-384; hsa-mir-3907; hsa-mir-3908; hsa-mir-3909; hsa-mir-3910-1; hsa-mir-3910-2; hsa-mir-3911; hsa-mir-3912; hsa-mir-3913-1; hsa-mir-3913-2; hsa-mir-3914-1; hsa-mir-3914-2; hsa-mir-3915; hsa-mir-3916; hsa-mir-3917; hsa-mir-3918; hsa-mir-3919; hsa-mir-3920; hsa-mir-3921; hsa-mir-3922; hsa-mir-3923; hsa-mir-3924; hsa-mir-3925; hsa-mir-3926-1; hsa-mir-3926-2; hsa-mir-3927; hsa-mir-3928; hsa-mir-3929; hsa-mir-3934; hsa-mir-3935; hsa-mir-3936; hsa-mir-3937; hsa-mir-3938; hsa-mir-3939; hsa-mir-3940; hsa-mir-3941; hsa-mir-3942; hsa-mir-3943; hsa-mir-3944; hsa-mir-3945; hsa-mir-409; hsa-mir-410; hsa-mir-411; hsa-mir-412; hsa-mir-421; hsa-mir-422a; hsa-mir-423; hsa-mir-424; hsa-mir-425; hsa-mir-4251; hsa-mir-4252; hsa-mir-4253; hsa-mir-4254; hsa-mir-4255; hsa-mir-4256; hsa-mir-4257; hsa-mir-4258; hsa-mir-4259; hsa-mir-4260; hsa-mir-4261; hsa-mir-4262; hsa-mir-4263; hsa-mir-4264; hsa-mir-4265; hsa-mir-4266; hsa-mir-4267; hsa-mir-4268; hsa-mir-4269; hsa-mir-4270; hsa-mir-4271; hsa-mir-4272; hsa-mir-4273; hsa-mir-4274; hsa-mir-4275; hsa-mir-4276; hsa-mir-4277; hsa-mir-4278; hsa-mir-4279; hsa-mir-4280; hsa-mir-4281; hsa-mir-4282; hsa-mir-4283-1; hsa-mir-4283-2; hsa-mir-4284; hsa-mir-4285; hsa-mir-4286; hsa-mir-4287; hsa-mir-4288; hsa-mir-4289; hsa-mir-429; hsa-mir-4290; hsa-mir-4291; hsa-mir-4292; hsa-mir-4293; hsa-mir-4294; hsa-mir-4295; hsa-mir-4296; hsa-mir-4297; hsa-mir-4298; hsa-mir-4299; hsa-mir-4300; hsa-mir-4301; hsa-mir-4302; hsa-mir-4303; hsa-mir-4304; hsa-mir-4305; hsa-mir-4306; hsa-mir-4307; hsa-mir-4308; hsa-mir-4309; hsa-mir-431; hsa-mir-4310; hsa-mir-4311; hsa-mir-4312; hsa-mir-4313; hsa-mir-4314; hsa-mir-4315-1; hsa-mir-4315-2; hsa-mir-4316; hsa-mir-4317; hsa-mir-4318; hsa-mir-4319; hsa-mir-432; hsa-mir-4320; hsa-mir-4321; hsa-mir-4322; hsa-mir-4323; hsa-mir-4324; hsa-mir-4325; hsa-mir-4326; hsa-mir-4327; hsa-mir-4328; hsa-mir-4329; hsa-mir-433; hsa-mir-4330; hsa-mir-448; hsa-mir-449a; hsa-mir-449b; hsa-mir-449c; hsa-mir-450a-1; hsa-mir-450a-2; hsa-mir-450b; hsa-mir-451; hsa-mir-452; hsa-mir-454; hsa-mir-455; hsa-mir-466; hsa-mir-483; hsa-mir-484; hsa-mir-485; hsa-mir-486; hsa-mir-487a; hsa-mir-487b; hsa-mir-488; hsa-mir-489; hsa-mir-490; hsa-mir-491; hsa-mir-492; hsa-mir-493; hsa-mir-494; hsa-mir-495; hsa-mir-496; hsa-mir-497; hsa-mir-498; hsa-mir-499; hsa-mir-500a; hsa-mir-500b; hsa-mir-501; hsa-mir-502; hsa-mir-503; hsa-mir-504; hsa-mir-505; hsa-mir-506; hsa-mir-507; hsa-mir-508; hsa-mir-509-1; hsa-mir-509-2; hsa-mir-509-3; hsa-mir-510; hsa-mir-511-1; hsa-mir-511-2; hsa-mir-512-1; hsa-mir-512-2; hsa-mir-513a-1; hsa-mir-513a-2; hsa-mir-513b; hsa-mir-513c; hsa-mir-514-1; hsa-mir-514-2; hsa-mir-514-3; hsa-mir-514b; hsa-mir-515-1; hsa-mir-515-2; hsa-mir-516a-1; hsa-mir-516a-2; hsa-mir-516b-1; hsa-mir-516b-2; hsa-mir-517a; hsa-mir-517b; hsa-mir-517c; hsa-mir-518a-1; hsa-mir-518a-2; hsa-mir-518b; hsa-mir-518c; hsa-mir-518d; hsa-mir-518e; hsa-mir-518f; hsa-mir-519a-1; hsa-mir-519a-2; hsa-mir-519b; hsa-mir-519c; hsa-mir-519d; hsa-mir-519e; hsa-mir-520a; hsa-mir-520b; hsa-mir-520c; hsa-mir-520d; hsa-mir-520e; hsa-mir-520f; hsa-mir-520g; hsa-mir-520h; hsa-mir-521-1; hsa-mir-521-2; hsa-mir-522; hsa-mir-523; hsa-mir-524; hsa-mir-525; hsa-mir-526a-1; hsa-mir-526a-2; hsa-mir-526b; hsa-mir-527; hsa-mir-532; hsa-mir-539; hsa-mir-541; hsa-mir-542; hsa-mir-543; hsa-mir-544; hsa-mir-544b; hsa-mir-545; hsa-mir-548a-1; hsa-mir-548a-2; hsa-mir-548a-3; hsa-mir-548aa-1; hsa-mir-548aa-2; hsa-mir-548b; hsa-mir-548c; hsa-mir-548d-1; hsa-mir-548d-2; hsa-mir-548e; hsa-mir-548f-1; hsa-mir-548f-2; hsa-mir-548f-3; hsa-mir-548f-4; hsa-mir-548f-5; hsa-mir-548g; hsa-mir-548h-1; hsa-mir-548h-2; hsa-mir-548h-3; hsa-mir-548h-4; hsa-mir-5481-1; hsa-mir-548i-2; hsa-mir-548i-3; hsa-mir-548i-4; hsa-mir-548j; hsa-mir-548k; hsa-mir-5481; hsa-mir-548m; hsa-mir-548n; hsa-mir-548o; hsa-mir-548p; hsa-mir-548q; hsa-mir-548s; hsa-mir-548t; hsa-mir-548u; hsa-mir-548v; hsa-mir-548w; hsa-mir-548x; hsa-mir-548y; hsa-mir-548z; hsa-mir-549; hsa-mir-550a-1; hsa-mir-550a-2; hsa-mir-550b-1; hsa-mir-550b-2; hsa-mir-551a; hsa-mir-551b; hsa-mir-552; hsa-mir-553; hsa-mir-554; hsa-mir-555; hsa-mir-556; hsa-mir-557; hsa-mir-558; hsa-mir-559; hsa-mir-561; hsa-mir-562; hsa-mir-563; hsa-mir-564; hsa-mir-566; hsa-mir-567; hsa-mir-568; hsa-mir-569; hsa-mir-570; hsa-mir-571; hsa-mir-572; hsa-mir-573; hsa-mir-574; hsa-mir-575; hsa-mir-576; hsa-mir-577; hsa-mir-578; hsa-mir-579; hsa-mir-580; hsa-mir-581; hsa-mir-582; hsa-mir-583; hsa-mir-584; hsa-mir-585; hsa-mir-586; hsa-mir-587; hsa-mir-588; hsa-mir-589; hsa-mir-590; hsa-mir-591; hsa-mir-592; hsa-mir-593; hsa-mir-595; hsa-mir-596; hsa-mir-597; hsa-mir-598; hsa-mir-599; hsa-mir-600; hsa-mir-601; hsa-mir-602; hsa-mir-603; hsa-mir-604; hsa-mir-605; hsa-mir-606; hsa-mir-607; hsa-mir-608; hsa-mir-609; hsa-mir-610; hsa-mir-611; hsa-mir-612; hsa-mir-613; hsa-mir-614; hsa-mir-615; hsa-mir-616; hsa-mir-617; hsa-mir-618; hsa-mir-619; hsa-mir-620; hsa-mir-621; hsa-mir-622; hsa-mir-623; hsa-mir-624; hsa-mir-625; hsa-mir-626; hsa-mir-627; hsa-mir-628; hsa-mir-629; hsa-mir-630; hsa-mir-631; hsa-mir-632; hsa-mir-633; hsa-mir-634; hsa-mir-635; hsa-mir-636; hsa-mir-637; hsa-mir-638; hsa-mir-639; hsa-mir-640; hsa-mir-641; hsa-mir-642a; hsa-mir-642b; hsa-mir-643; hsa-mir-644; hsa-mir-645; hsa-mir-646; hsa-mir-647; hsa-mir-648; hsa-mir-649; hsa-mir-650; hsa-mir-651; hsa-mir-652; hsa-mir-653; hsa-mir-654; hsa-mir-655; hsa-mir-656; hsa-mir-657; hsa-mir-658; hsa-mir-659; hsa-mir-660; hsa-mir-661; hsa-mir-662; hsa-mir-663; hsa-mir-663b; hsa-mir-664; hsa-mir-665; hsa-mir-668; hsa-mir-670; hsa-mir-671; hsa-mir-675; hsa-mir-676; hsa-mir-708; hsa-mir-7-1; hsa-mir-711; hsa-mir-718; hsa-mir-7-2; hsa-mir-720; hsa-mir-7-3; hsa-mir-744; hsa-mir-758; hsa-mir-759; hsa-mir-760; hsa-mir-761; hsa-mir-762; hsa-mir-764; hsa-mir-765; hsa-mir-766; hsa-mir-767; hsa-mir-769; hsa-mir-770; hsa-mir-802; hsa-mir-873; hsa-mir-874; hsa-mir-875; hsa-mir-876; hsa-mir-877; hsa-mir-885; hsa-mir-887; hsa-mir-888; hsa-mir-889; hsa-mir-890; hsa-mir-891a; hsa-mir-891b; hsa-mir-892a; hsa-mir-892b; hsa-mir-9-1; hsa-mir-9-2; hsa-mir-920; hsa-mir-921; hsa-mir-922; hsa-mir-924; hsa-mir-92a-1; hsa-mir-92a-2; hsa-mir-92b; hsa-mir-93; hsa-mir-9-3; hsa-mir-933; hsa-mir-934; hsa-mir-935; hsa-mir-936; hsa-mir-937; hsa-mir-938; hsa-mir-939; hsa-mir-940; hsa-mir-941-1; hsa-mir-941-3; hsa-mir-941-4; hsa-mir-942; hsa-mir-943; hsa-mir-944; hsa-mir-95; hsa-mir-96; hsa-mir-98; hsa-mir-99a; hsa-mir-99b.ebv-miR-BHRFI-1, ebv-miR-BHRFI-2*, ebv-miR-BHRFI-2, ebv-miR-BHRFI-3, ebv-miR-BARTI-5p, ebv-miR-BART2, hcmv-miR-UL22A, hcmv-miR-UL22A*, hcmv-miR-UL36, hcmv-miR-UL112, hcmv-miR-UL148D, hcmv-miR-US5-1, hcmv-miR-US5-2, hcmv-miR-US25-I, hcmv-miR-US25-2-5p, hcmv-miR-US25-2-3p, hcmv-miR-US33, kshv-miR-K12-10a, kshv-miR-K12-10b, kshv-miR-K12-II, kshv-miR-K12-I, kshv-miR-K12-2, kshv-miR-K12-9*, kshv-miR-K12-9, kshv-miR-K12-8, kshv-miR-K12-7, kshv-miR-K12-6-5p, kshv-miR-K12-6-3p, kshv-miR-K12-5, kshv-miR-K12-4-5p, kshv-miR-K12-4-3p, kshv-miR-K12-3, kshv-miR-K12-3*, hcmv-miR-US4, hcmv-miR-UL70-5p, hcmv-20 miR-UL70-3p, ebv-miR-BART3-5p, ebv-miR-BART3-3p, ebv-miR-BART4, ebv-miR-BART5, ebv-miR-BART6-5p, ebv-miR-BART6-3p, ebv-miR-BART7, ebv-miR-BART8-5p, ebv-miR-BART8-3p, ebv-miR-BART9, ebv-miR-BARTIO, ebv-miR-BARTII-5p, ebv-miR-BARTII-3p, ebv-miR-BART12, ebv-miR-BART13, ebv-miR-BART14-5p, ebv-miR-BART14-3p, kshv-miR-25 K12-12, ebv-miR-BART15, ebv-miR-BART16, ebv-miR-BART17-5p, ebv-miR-BART17-3p, ebv-miR-BART18, ebv-miR-BART19, ebv-miR-BART20-5p, ebv-miR-BART20-3p, and hsvl-miR-HI.

### D. miRNA Clusters

Recently, following the discovery of a number of miRNA genes, some groups indicated that clustered miRNAs work in combination to accomplish their function throughout many biological processes. As noted above, many miRNAs are encoded in miRNA gene clusters. As used herein, the terms “miRNA gene cluster” and “miRNA cluster” are used interchangeably and refer to a genomic locus or transcription unit that encodes two or more miRNA genes.

For example, expression of the mir-143 cluster is down-regulated in colon cancer as well as in some other cancers cell lines (Michael et al., Reduced accumulation of specific microRNAs in colorectal neoplasia, Mol. Cancer Res. 1 (2003) 882-891). The mir-430 cluster was shown to regulate neurogenesis in zebrafish (Giraldez et al., MicroRNAs regulate brain morphogenesis in zebrafish, Science 308 (2005) 833-838). The mir-17 cluster modulates E2F1 expression and might be a potential human oncogene (O'Donnell et al., c-Myc-regulated microRNAs modulate E2F1 expression, Nature 435 (2005) 839-843), and the mir-15a cluster can induce leukemia cell apoptosis by targeting BCL2 (Cimmino et al., miR-15 and miR-16 induce apoptosis by targeting BCL2, Proc. Natl. Acad. Sci. 102 (2005) 13944-13949).

In a recent study, the proportion of clustered miRNAs in humans goes far beyond what was previously envisioned (Altuvia et al., Clustering and conservation patterns of human microRNAs, Nucleic Acids Res. 33 (2005) 2697-2706). This natural genomic organization pattern of miRNA genes provides internal mechanisms for them to function in coordination, but to date, no such mechanisms have been described and the role of clusters in regulating development and disease is poorly understood. In another recent study Yu and colleagues performed profiling of a subset of clustered miRNAs in different types of leukemic cells lines (Yu et al., Human microRNA clusters: Genomic organization and expression profile in leukemia cell lines, 349 B.B.R.C.: 59-68). Although Yu et al., discovered that multiple miRNAs demonstrate activity in clusters expressed in leukemic cells, the analysis ends there.

Several groups have taken genomic approaches to identify miRNA clusters (see, e.g., Zhang et al., Sci China Ser C-Life Sci, (2009) 52: 261-266; Yu et al., 2006; Altuvia et al., 2005). Altuvia and colleagues used a set of 207 human miRNAs to identify 31 miRNA clusters. Yu and colleagues used a dataset of 326 human miRNA genes and identified 148 miRNAs organized into 51 clusters. The present invention used the most current entries from the miRNA database at the Sanger Center in the United Kingdom. Using a dataset of 709 miRNA genes, 224 miRNAs organized into 73 clusters were identified. The 73 clusters contain all the clusters of Yu et al., 2006. Table 1 shows illustrative target miRNA clusters, chromosome locations, and organization of paralogous groups.

TABLE 1
miRNA clusters

| | Cluster Name | |
|---|---|---|
| Paralog | (based on most 5′ | |
| group | miRNA of cluster) | miRNA genes in cluster from 5′ to 3′ |
| 1 | has-let7a-1 cluster | hsa-let-7a-1, hsa-let-7f-1, hsa-let-7d |
| | hsa-let-7a-2 cluster | hsa-let-7a-2, hsa-mir-100 |
| | hsa-let-7f-2 cluster | hsa-let-7f-2, hsa-mir-98 |
| 2 | hsa-mir-29b-2 cluster | hsa-mir-29b-2, hsa-mir-29c |
| | hsa-mir-29b-1 cluster | hsa-mir-29b-1, hsa-mir-29a |
| 3 | hsa-mir-15b cluster | hsa-mir-15b, hsa-mir-16-2 |
| | hsa-mir-15a cluster | hsa-mir-15a, hsa-mir-16-1 |
| 4 | hsa-mir-181a-1 | hsa-mir-181a-1, hsa-mir-181b-1 |
| | cluster | |
| | hsa-mir-181a-2 | hsa-mir-181a-2, hsa-mir-181b-2 |
| | cluster | |
| | hsa-mir-181c cluster | hsa-mir-181c, hsa-mir-181d |
| 5 | hsa-mir-106b cluster | hsa-mir-106b, hsa-mir-93, hsa-mir- |
| | | 25 |
| | hsa-mir-17 cluster | hsa-mir-17, hsa-mir-18a, hsa-mir- |
| | | 19a, hsa-mir-20a, hsa-mir-19b-1, |
| | | hsa-mir-92a-1 |
| | hsa-mir-106a cluster | hsa-mir-106a, hsa-mir-18b, hsa-mir- |
| | | 20b, hsa-mir-19b-2, hsa-mir-92a-2, |
| | | hsa-mir-363 |
| 6 | hsa-mir-99b cluster | hsa-mir-99b, hsa-let-7e, hsa-mir- |
| | | 125a |
| | hsa-mir-99a cluster | hsa-mir-99a, hsa-let-7c |
| 7 | hsa-mir-23b cluster | hsa-mir-23b, hsa-mir-27b, hsa-mir- |
| | | 24-1 |
| | hsa-mir-23a cluster | hsa-mir-23a, hsa-mir-27a, hsa-mir- |
| | | 24-2 |
| 8 | hsa-mir-200b cluster | hsa-mir-200b, hsa-mir-200a, hsa- |
| | | mir-429 |
| | hsa-mir-200c cluster | hsa-mir-200c, hsa-mir-141 |
| 9 | hsa-mir-518e cluster | hsa-mir-518e, hsa-mir-518a-1 |
| | hsa-mir-518d cluster | hsa-mir-518d, hsa-mir-516b-1, hsa- |
| | | mir-518a-2, hsa-mir-517c, hsa-mir- |
| | | 520h |
| | hsa-mir-515-1 cluster | hsa-mir-515-1, hsa-mir-519e, hsa- |
| | | mir-520f |
| | hsa-mir-515-2 cluster | hsa-mir-515-2, hsa-mir-519c, hsa- |
| | | mir-1283-1 |
| | hsa-mir-521-1 cluster | hsa-mir-521-1, hsa-mir-522, hsa- |
| | | mir-519a-1, hsa-mir-527, hsa-mir- |
| | | 516a-1 |
| | hsa-mir-526b cluster | hsa-mir-526b, hsa-mir-519b, hsa- |
| | | mir-525, hsa-mir-523, hsa-mir-518f, |
| | | hsa-mir-520b, hsa-mir-518 |
| | hsa-mir-526a-1 | hsa-mir-526a-1, hsa-mir-520c, hsa- |
| | cluster | mir-518c, hsa-mir-524, hsa-mir- |
| | | 517a, hsa-mir-519d |
| | hsa-mir-520d cluster | hsa-mir-520d, hsa-mir-517b, hsa- |
| | | mir-520g |
| Clusters | | |
| with no | | |
| identified | | |
| paralogs | | |
| n/a | hsa-mir-30e cluster | hsa-mir-30e, hsa-mir-30c1 |
| n/a | hsa-mir-199a-2 | hsa-mir-199a-2, hsa-mir-214 |
| | cluster | |
| n/a | hsa-mir-194-1 cluster | hsa-mir-194-1, hsa-mir-215 |
| n/a | hsa-mir-216a cluster | hsa-mir-216a, hsa-mir-217 |
| n/a | hsa-mir-191 cluster | hsa-mir-191, hsa-mir-425 |
| n/a | hsa-mir-302b cluster | hsa-mir-302b, hsa-mir-302c, hsa- |
| | | mir-302a, hsa-mir-302d, hsa-mir- |
| | | 367 |
| n/a | hsa-mir-449c cluster | hsa-mir-449c, hsa-mir-449b, hsa- |
| | | mir-449a |
| n/a | hsa-mir-143 cluster | hsa-mir-143, hsa-mir-145 |
| n/a | hsa-mir-489 cluster | hsa-mir-489, hsa-mir-653 |
| n/a | hsa-mir-183 cluster | hsa-mir-183, hsa-mir-96, hsa-mir- |
| | | 182 |
| n/a | hsa-mir-875 cluster | hsa-mir-875, hsa-mir-599 |
| n/a | hsa-mir-1234 cluster | hsa-mir-1234, hsa-mir-939 |
| n/a | hsa-mir-194-2 cluster | hsa-mir-194-2, hsa-mir-192 |
| n/a | hsa-mir-34b cluster | hsa-mir-34b, hsa-mir-34c |
| n/a | hsa-mir-493 cluster | hsa-mir-493, hsa-mir-337, hsa-mir- |
| | | 665, hsa-mir-431, hsa-mir-433, hsa- |
| | | mir-127, hsa-mir-432, hsa-mir-136 |
| n/a | hsa-mir-379 cluster | hsa-mir-379, hsa-mir-411, hsa-mir- |
| | | 299, hsa-mir-380, hsa-mir-1197, |
| | | hsa-mir-323, hsa-mir-758, hsa-mir- |
| | | 329-1, hsa-mir-329-2 |
| n/a | hsa-mir-376c cluster | hsa-mir-37c, hsa-mir-376a-2, hsa- |
| | | mir-654, hsa-mir-376b, hsa-mir- |
| | | 376a-1, hsa-mir-300 |
| n/a | hsa-mir-381 cluster | hsa-mir-381, hsa-mir-487b, hsa-mir- |
| | | 539, hsa-mir-889, hsa-mir-544, hsa- |
| | | mir-655 |
| n/a | hsa-mir-382 cluster | hsa-mir-382, hsa-mir-134, hsa-mir- |
| | | 668, hsa-mir-485, hsa-mir-453 |
| n/a | hsa-mir-541 cluster | hsa-mir-541, hsa-mir-409, hsa-mir- |
| | | 412, hsa-mir-369, hsa-mir-410, hsa- |
| | | mir-656 |
| n/a | hsa-mir-193b cluster | hsa-mir-193b, hsa-mir-365-1 |
| n/a | hsa-mir-212 cluster | hsa-mir-212, hsa-mir-132 |
| n/a | hsa-mir-497 cluster | hsa-mir-497, hsa-mir-195 |
| n/a | hsa-mir-144 cluster | hsa-mir-144, hsa-mir-451 |
| n/a | hsa-mir-338 cluster | hsa-mir-338, hsa-mir-657 |
| n/a | hsa-mir-498 cluster | hsa-mir-498, hsa-mir-520e |
| n/a | hsa-mir-516b-2 | hsa-mir-516b-2, hsa-mir-526a-2 |
| | cluster | |
| n/a | hsa-mir-516a-2 | hsa-mir-516a-2, hsa-mir-519a-2 |
| | cluster | |
| n/a | hsa-mir-371 cluster | hsa-mir-371, hsa-mir-372, hsa-mir- |
| | | 373 |
| n/a | hsa-mir-298 cluster | hsa-mir-298, hsa-mir-296 |
| n/a | hsa-mir-941-1 cluster | hsa-mir-941-1, hsa-mir-941-2, hsa- |
| | | mir-941-3 |
| n/a | hsa-mir-301b cluster | hsa-mir-301b, hsa-mir-130b |
| n/a | hsa-let-7a-3 cluster | hsa-let-7a-3, hsa-let-7b |
| n/a | hsa-mir-221 cluster | hsa-mir-221, hsa-mir-222 |
| n/a | hsa-mir-532 cluster | hsa-mir-532, hsa-mir-188, hsa-mir- |
| | | 500, hsa-mir-62, hsa-mir-501, hsa- |
| | | mir-660, hsa-mir-502 |
| n/a | hsa-mir-374b cluster | hsa-mir-374b, hsa-mir-421 |
| n/a | hsa-mir-374a cluster | hsa-mir-374a, hsa-mir-545 |
| n/a | hsa-mir-542 cluster | hsa-mir-542, hsa-mir-450a-2, hsa- |
| | | mir-450a-1, hsa-mir-450b |
| n/a | hsa-mir-424 cluster | hsa-mir-424, hsa-mir-503 |
| n/a | hsa-mir-888 cluster | hsa-mir-888, hsa-mir-890 |
| n/a | hsa-mir-892b cluster | hsa-mir-892b, hsa-mir-892a |
| n/a | hsa-mir-507 cluster | hsa-mir-507, hsa-mir-506 |
| n/a | hsa-mir-509-1 cluster | hsa-mir-509-1, hsa-mir-509-3, hsa- |
| | | mir-509-2 |
| n/a | hsa-mir-514-3 cluster | hsa-mir-514-3, hsa-mir-514-2, hsa- |
| | | mir-514-1, hsa-mir-510 |
| n/a | hsa-mir-452 cluster | hsa-mir-452, hsa-mir-224 |
| n/a | hsa-mir-105-2 cluster | hsa-mir-105-2, hsa-mir-767, hsa- |
| | | mir-105-1 |

The numbering of miRNA genes is simply sequential. The names/identifiers are in the form hsa-mir-121. The first three letters signify the organism; “hsa” refers to Homo sapiens. The “mir-” prefix refers to the miRNA gene and also to the predicted stem-loop portion of the primary transcript. Distinct precursor sequences and genomic loci that express identical mature sequences get names of the form hsa-mir-121-1 and hsa-mir-121-2. Distinct precursor sequences and genomic loci that express nearly identical sequences bar one or two nucleotides are annotated with an additional lower case letter, e.g., for example hsa-mir-121a and hsa-mir-121b.

### E. miRNA mimetics

The present invention further contemplates, in part, oligomers that are referred to as miRNA mimetics. The term “microRNA mimetic” or “miRNA mimetic” or “miRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., pri- or pre-miRNAs). miRNA mimics can be comprised of modified or unmodified RNA, DNA, RNA-DNA hybrids, or alternative nucleic acid chemistries (e.g., locked nucleic acids (LNAs) or 2′-O, 4′-C ethylene bridged nucleic acids (ENAs)). For mature, double stranded miRNA mimics, the length of the duplex region can vary between about 16 and 31 nucleotides and chemical modification patterns can include the following: the sense strand contains 2′-O-methyl modifications of nucleotides 1 and 2 (counting from the 5′ end of the sense oligonucleotide) and all the Cs and Us. In addition, the sense strand can comprise a conjugate that enhances functionality, delivery, or specificity. The antisense strand modifications may comprise 2′ F modification of all the Cs and Us, phosphorylation of the 5′ end of the oligonucleotide, and stabilized internucleotide linkages associated with a 2 nucleotide 3′ overhang.

### F. piRNA Mimetics

The present invention further contemplates, in part, oligomers that are referred to as piRNA mimetics. The term “piRNA mimetics” refers to synthetic non-coding RNAs that are capable of entering the RNAi pathway and regulating gene expression. piRNA mimics imitate the function of endogenous Piwi-interacting RNAs, a class of small RNAs that are believed to be involved in transcriptional silencing (see Lau, N. C. et al. (2006) Science, 313:305-306).

### G. shRNAs

The present invention further contemplates, in part, oligomers that are referred to as shRNAs. An shRNA is a double-stranded structure formed by a single self-complementary RNA strand. RNA duplex formation may be initiated either inside or outside the cell. Inhibition is sequence-specific in that nucleotide sequences corresponding to the duplex region of the RNA are targeted for genetic inhibition. shRNA constructs containing a nucleotide sequence identical to a portion, of either coding or non-coding sequence, of the target gene are preferred for inhibition. RNA sequences with insertions, deletions, and single point mutations relative to the target sequence have also been found to be effective for inhibition. Because 100% sequence identity between the RNA and the target gene is not required to practice the present invention, the invention has the advantage of being able to tolerate sequence variations that might be expected due to genetic mutation, strain polymorphism, or evolutionary divergence. Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing). In certain preferred embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size.

An endogenous RNA polymerase of the cell may mediate transcription of an shRNA encoded in a nucleic acid construct. The shRNA construct may also be synthesized by a bacteriophage RNA polymerase (e.g., T3, T7, SP6) that is expressed in the cell. In preferred embodiments, expression of an shRNA is regulated by an RNA polymerase III promoters; such promoters are known to produce efficient silencing. While essentially any PolIII promoters may be used, desirable examples include the human U6 snRNA promoter, the mouse U6 snRNA promoter, the human and mouse H1 RNA promoter and the human tRNA-val promoter. A U6 snRNA leader sequence may be appended to the primary transcript; such leader sequences tend to increase the efficiency of sub-optimal shRNAs while generally having little or no effect on efficient shRNAs. For transcription from a transgene in vivo, a regulatory region (e.g., promoter, enhancer, silencer, splice donor and acceptor, polyadenylation) may be used to regulate expression of the shRNA strand (or strands). Inhibition may be controlled by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus. The use and production of an expression construct are known in the art (see also WO 97/32016; U.S. Pat. Nos. 5,593,874, 5,698,425, 5,712,135, 5,789,214, and 5,804,693; and the references cited therein).

An shRNA construct may be designed with 29 bp helices following a U6 snRNA leader sequence with the transcript being produced by the human U6 snRNA promoter. This transcription unit may be delivered via a Murine Stem Cell Virus (MSCV)-based retrovirus, with the expression cassette inserted downstream of the packaging signal. Further information on the optimization of shRNA constructs may be found, for example, in the following references: Paddison, P. J., A. A. Gaudy, and G. J. Hannon, Stable suppression of gene expression by RNAi in mammalian cells. Proc Natl Acad Sci USA, 2002. 99(3): p. 1443-8; 13. Brummelkamp, T. R., R. Bemards, and R. Agami, A System for Stable Expression of Short Interfering RNAs in Mammalian Cells. Science, 2002. 21: p. 21; Kawasaki, H. and K. Taira, Short hairpin type of dsRNAs that are controlled by tRNA(Val) promoter significantly induce RNAi-mediated gene silencing in the cytoplasm of human cells. Nucleic Acids Res, 2003. 31(2): p. 700-7, Lee, N. S., et al., Expression of small interfering RNAs targeted against HIV-1 rev transcripts in human cells. Nat Biotechnol, 2002. 20(5): p. 500-5; Miyagishi, M. and K. Taira, U6 promoter-driven siRNAs with four uridine 3′ overhangs efficiently suppress targeted gene expression in mammalian cells. Nat Biotechnol, 2002. 20(5): p. 497-500; Paul, C P., et al., Effective expression of small interfering RNA in human cells. Nat Biotechnol, 2002. 20(5): p. 505-8.

### H. Anti-miRNAs

In particular embodiments, the present invention contemplates, in part, oligomers that are anti-miRNA molecules. As used herein, the term “anti-miRNA molecule” or “anti-miRNA” refers to an oligomer that interferes with the expression and/or activity of a pre-miRNA, pri-miRNA, and/or miRNAs.

### I. Oligomers

The present invention contemplates, in part, an anti-miRNA molecule comprising an oligomer or oligomeric compound. As used herein, the terms “oligomer,” “oligomeric,” or equivalents thereof, refer to polymeric structures that hybridize to target RNA sequences present in a cell. Oligomers can be single stranded or double stranded and include an anti-miRNA, an sRNA, an shRNA, a piRNA mimetic, and a miRNA mimetic.

The target RNA sequence includes, but is not limited to pre-mRNA, mRNA, pre-miRNA, pri-miRNA, and miRNA. A target sequence present in a miRNA or a pri-miRNA transcript each which may be transcribed by a miRNA gene promoter or transcribed by a host gene promoter, in which case the pri-miRNA transcript is included in the pre-mRNA or mRNA sequence of the host gene.

Oligomers of the invention comprise one or more of the following features: minor groove binding moieties (MGBs), modified or unmodified nucleotides and nucleosides, nucleotide analogs, nucleoside analogs, nucleotide mimetics, each of which can comprise various modified sugars or modified internucleotide/internucleoside linkages, conjugates (e.g., cholesterol), and linkers that facilitate covalently binding the conjugates and/or MGBs to the oligomer. In one embodiment, the term “oligomer” refers to an oligomer comprising one or more MGB moieties. In one embodiment, the term “oligomer” refers to a plurality of monomeric units comprising one or more MGB moieties. An oligomer of the present invention has increased biodistribution compared to unconjugated oligomers and/or molecules that lack MGBs.

As used herein, the term “biodistribution” refers to the cellular and/or tissue and/or organ distribution of an oligomer that is administered or delivered to a subject. As used herein, the terms “promoting,” “enhancing,” “stimulating,” or “increasing” generally refer to the ability of an oligomer of the invention that has a greater or broader tissue distribution, as compared to the response caused by either vehicle or a control molecule/composition. Various methods known to the skilled artisan can be used to assessed and increased biodistribution of cells or tissues, including, but not limited to: nuclear medicine, whole body autoradiography, micro-autoradiography; phosphor imaging, cryo-imaging, nano-secondary ion mass spectroscopy (nanoSIMS), matrix-assisted laser desorption imaging (MALDI-MS), radiography (X-Ray), magnetic resonance imaging (MRI), computed tomography (CT), micro-ultrasound single photon emission CT (SPECT), positron emission tomography (PET) and the like. An increased biodistribution of the inventive oligomer, includes an at least 5%, 10%, 20%, 30%, 40%, 50%, 60%, 70%, 80%, 90%, 100%, 125%, 150%, 175%, 200%, or greater increase compared to a vehicle or a control molecule/composition. An “increased” or “enhanced” tissue distribution or biodistribution is typically a “statistically significant” increase, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the distribution of a vehicle or a control composition.

Without wishing to be bound to any particular theory, conjugation of one or more MGBs to an oligomer, as described elsewhere herein, can increase the hybridization stability and stringency of hybridization between an oligomer and its target RNA nucleic acid sequence compared to an oligomer lacking an MGB. In addition, conjugation of one or more MGBs to an oligomer, as described elsewhere herein, results in increased tissue distribution and bioavailability of the oligomer. These characteristics can be important in improving the delivery and efficacy of systemically administered oligomers.

In one embodiment, the present invention contemplates, in part, that oligomers comprise modifications that facilitate the ability of the oligomer to cross cell, tissue, or organ barriers to nucleic acid based-drug entry compared to unmodified oligomers. In one, non-limiting example, an oligomer comprising conjugated to or linked to one or more MGBs has a substantially increased frequency of penetrating a cell, tissue, or organ barrier compared to an oligomer that lacks an MGB moiety.

In one embodiment, an oligomer of the present invention demonstrates a surprising and unexpected property of increased biodistribution. In a preferred embodiment, an oligomer inhibits gene expression or miRNA expression a wide number of cell or tissue types and/or organs than an anti-miRNA that lacks an MGB moiety.

In particular preferred embodiments, an oligomer inhibits the expression of the target RNAs in two or more, three or more, four or more, five or more, six or more, seven or more, or eight or more cell or tissue types and/or organs.

In one embodiment, an oligomer hybridizes to a target miRNA sequence that comprises the guide strand miRNA sequence. In another embodiment an oligomer hybridizes to a target miRNA sequence that comprises the passenger or star strand miRNA sequence.

In certain embodiments, an oligomer of the present invention decreases the expression or activity of 1, 2, 3, 4, 5, 6, 7, 8, 9, 10, 11, 12, 13, 14, 15, 16, 17, 18, 19, 20 or more RNAs in 1, 2, 3, 4, 5, 6, 7, 8, 9, 10 or more cell types, tissues, or organs. In additional embodiments, the present invention provides methods of using oligomers to inhibit the RNA expression and/or activity of an RNA in every cell type, tissue, or organ. In other embodiments, the present invention provides methods of using oligomers to inhibit the RNA expression or activity of an RNA in a single type of cell, single tissue, or single organ.

In certain embodiments, oligomers having increased cell, tissue, or organ distribution comprise at least one MGB moiety and any combination of the following features, without limitation: modified or unmodified nucleotides and nucleosides, nucleotide analogs, nucleoside analogs, nucleotide mimetics, each of which can comprise various modified sugars or modified internucleotide/internucleoside linkages, conjugates (e.g., cholesterol), and linkers that facilitate covalently binding the conjugates and/or MGBs to the oligomer. Each of the features discussed in further detail below.

In various embodiments, an oligomer comprises a plurality of covalently linked monomeric subunits in which each linked monomeric subunit comprises a heterocyclic base moiety and a sugar moiety or sugar surrogate. The monomeric units can be linked by various internucleotide/internucleoside linkages known in the art or described herein. Each of the components of the oligomer can be present in a modified or unmodified form, independent of the other monomeric units in the oligomer. Illustrative monomeric units of oligomers include, but are not limited to, modified or unmodified nucleotides and nucleosides, nucleotide analogs, nucleoside analogs, nucleotide mimetics, each of which can comprise various modified sugars or modified internucleotide/internucleoside linkages. Accordingly, oligomers of the present invention comprise modified and/or non-modified monomeric units in any suitable combination as long as the oligomers retain or have an increase in the desired activity, e.g., hybridization specificity, hybridization affinity, nuclear entry, and anti-RNA catalytic activity.

In particular illustrative embodiments, a modified oligomer is preferred over an unmodified oligomer. Without wishing to be bound by any particular theory, modified oligomers are preferred over native forms in particular embodiments because of desirable properties such as, for example, increased nuclear entry, increased hybridization affinity to a target nucleic acid, increased hybridization specificity to a target nucleic acid, enhanced cellular uptake, increased resistance to nucleases, and decreased therapeutic toxicity. As used herein, the term “modification” includes substitution and/or any change to internucleoside linkages, sugar moieties, base moieties, conjugation to conjugates, linkers, and MGB moieties such as those described below. In some embodiments, the oligomer is completely modified, and in other embodiments, the oligomer is partially modified. Moreover, an oligomer comprising one or more MGB moieties substantially enhances delivery of the oligomer to cells, tissues, and/or organs, hybridization affinity to a target nucleic acid, hybridization specificity to a target nucleic acid, cellular uptake, tissue uptake, organ uptake, resistance to nucleases, and substantially decreases therapeutic toxicity compared to a modified oligomer that is not conjugated or linked an MGB moiety.

The oligomers in accordance with this invention can comprise from about 6 to about 100 monomeric subunits, about 10 to about 50 monomeric subunits, about 12 to about 35 monomeric subunits, about 15 to about 25 monomeric subunits, about 18 to about 23 monomeric subunits, or any intervening number of monomeric subunits. One of ordinary skill in the art will appreciate that the invention includes oligomers of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 subunits in length.

As used herein, the term “hybridization” refers to the pairing of an oligomer to a complementary nucleic acid sequence (e.g., the target RNA sequence). Exemplary nucleic acid sequences that comprise the target RNA sequence includes, but is not limited to, a pre-mRNA sequence, an mRNA sequence, a pri-miRNA sequence, a pre-miRNA sequence, a mature miRNA sequence, complements thereof, and fragments or portions thereof. In a one embodiment, a pre-mRNA or mRNA comprising a pri-miRNA transcript comprises the target miRNA sequence. In another embodiment, pri-miRNA transcript comprises the target miRNA sequence. In the present invention, the mechanism of pairing involves hydrogen bonding, which may be Watson-Crick, Hoogsteen or reversed Hoogsteen hydrogen bonding, between an oligomer of the invention and the target miRNA sequence.

As used herein, the term “specifically hybridizable” means that there is a sufficient degree of complementarity to avoid non-specific binding of the oligomer to non-target nucleic acids under conditions in which specific hybridization is desired, i.e., under physiological conditions in the case of in vivo or ex vivo assays or therapeutic treatment, and under standard assay conditions in the case of in vitro assays. In one embodiment, an oligomer that is specifically hybridizable to a target miRNA sequence interferes with the normal function of the target miRNA. Consequently, the oligomer alters the activity and/or disrupts the function of a miRNA.

One having ordinary skill in the art would appreciate that the specificity of oligomers of the present invention can be modulated through any of the modifications discussed herein, e.g., modified nucleobases, modified sugars. In addition to these modifications, an oligomer comprising an oligomer, e.g., a plurality of oligomeric units, linked to one or more MGB moieties substantially increases the hybridization specificity to the target miRNA compared to an oligomer that lacks an MGB moiety.

As used herein, the term “stringent hybridization” or “stringent conditions” refers to conditions under which an oligomer of the invention will specifically hybridize to its target RNA sequence. Stringent conditions are sequence-dependent and will vary with different circumstances and in the present context; “stringent conditions” under which oligomers hybridize to a target RNA are determined by the nature and composition of the oligomers and the assays in which they are being investigated. One having ordinary skill in the art will understand variability in the experimental protocols and be able to determine when conditions are optimal for stringent hybridization with minimal non-specific hybridization events. Moreover, an oligomer comprising one or more MGB moieties substantially increases the stringency of hybridization and minimizes non-specific hybridization events compared to an oligomer that lacks an MGB moiety.

As used herein, the term “complementarity” refers to the capacity for precise pairing of one nucleobase with another. For example, if a monomeric subunit at a certain position of an oligomer is capable of hydrogen bonding with a monomeric subunit at a certain position of a target RNA sequence, then the position is considered to be a complementary position. In contrast, a position is considered “non-complementary” when monomeric subunits are not capable of hydrogen bonding. The oligomer is substantially complementary to a target RNA sequence if a sufficient number of complementary positions in each molecule are occupied by monomeric subunits that can hydrogen bond with each other. As used herein, the term “substantially complementary” refers to a sufficient degree of precise pairing over a sufficient number of monomeric subunits such that stable and specific binding occurs between the oligomer and a target RNA.

In particular illustrative embodiments, an oligomer is completely complementary to a target RNA sequence. In certain embodiments, an oligomer is about 75% to about 95%, about 80% to about 95%, about 85% to about 95%, or about 90% to about 95% complementary to a target RNA sequence. In another embodiment, an oligomer is about 75%, about 80%, about 85%, about 90%, or about 95% or more complementary to a target RNA sequence.

Accordingly, an oligomer can be about 95% identical, about 90% identical, about 85% identical, about 80% identical, or to a target RNA sequence.

In a further embodiment, the oligomer can hybridize over one or more segments such that intervening or adjacent segments are not involved in the hybridization (e.g., a bulge, a loop structure or a hairpin structure). As used herein, the term “mismatch” refers to a non-complementary base between the oligomer and the target RNA.

In particular embodiments, non-complementary or mismatch positions may be tolerated between an oligomer and the target RNA provided that the oligomer remains substantially complementary to the rest of the target RNA sequence. Oligomers of the invention can comprise an oligonucleotide compound with 1, 2, or 3 mismatches to a target RNA sequence without causing a significant decrease in the ability of the oligomer to modulate the expression or activity of a target RNA. In preferred embodiments, mismatches are preferred outside of the region of the oligomer which is complementary to the seed sequence of the target RNA. In another preferred embodiment, the oligomer contains 0, 1 or 2 mismatches to the target RNA sequence. In a more preferred embodiment, the oligomer contains at most 1 mismatch to the target RNA sequence. In a further preferred embodiment, the oligomer hybridizes to a target RNA sequence that comprises the seed sequence.

In various embodiments, oligomers of the invention can be formed as composite structures of two or more modified or unmodified nucleotides and nucleosides, nucleotide analogs, nucleoside analogs, nucleotide mimetics, as described herein. Such oligomers have also been referred to in the art as hybrids, hemimers, gapmers or inverted gapmers. Representative U.S. patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

1. Nucleobases

The present invention contemplates, in part, that oligomers can comprise one or more modified or non-modified nucleotides and/or nucleosides. Oligomers of the present invention comprising one or more modified or non-modified nucleotides and/or nucleosides can have increased resistance to nucleases, increased binding affinity to a target nucleic acid and other beneficial biological properties.

As used herein the term “nucleotide” refers to a heterocyclic nitrogenous base in N-glycosidic linkage with a phosphorylated sugar. Nucleotides are understood to include natural bases, and a wide variety of art-recognized modified bases. Such bases are generally located at the 1′ position of a nucleotide sugar moiety. Nucleotides generally comprise a base, sugar and a phosphate group. In ribonucleic acid (RNA), the sugar is a ribose, and in deoxyribonucleic acid (DNA) the sugar is a deoxyribose, i.e., a sugar lacking a hydroxyl group that is present in ribose. Exemplary natural nitrogenous bases include the purines, adenosine (A) and guanidine (G), and the pyrimidines, cytidine (C) and thymidine (T) (or in the context of RNA, uracil (U)). The C-1 atom of deoxyribose is bonded to N-1 of a pyrimidine or N-9 of a purine. Nucleotides are usually mono, di- or triphosphates. The nucleotides can be unmodified or modified at the sugar, phosphate and/or base moiety, (also referred to interchangeably as nucleotide analogs, nucleotide derivatives, modified nucleotides, non-natural nucleotides, and non-standard nucleotides; see for example, WO 92/07065 and WO 93/15187). Examples of modified nucleic acid bases are summarized by Limbach et al., (1994, Nucleic Acids Res. 22, 2183-2196).

In particular illustrative embodiments, the oligomer of the invention comprises one or more modified nucleotides. In certain embodiments, all the nucleotides are modified, and in other certain embodiments, none of the nucleotides are modified. In various embodiments, the oligomer comprises about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or 100% modified nucleotides.

A nucleotide may also be regarded as a phosphate ester of a nucleoside, with esterification occurring on the hydroxyl group attached to C-5 of the sugar. As used herein, the term “nucleoside” refers to a heterocyclic nitrogenous base in N-glycosidic linkage with a sugar. Nucleosides are recognized in the art to include natural bases, and also to include well known modified bases. Such bases are generally located at the 1′ position of a nucleoside sugar moiety. Nucleosides generally comprise a base and sugar group. The nucleosides can be unmodified or modified at the sugar, and/or base moiety, (also referred to interchangeably as nucleoside analogs, nucleoside derivatives, modified nucleosides, non-natural nucleosides, or non-standard nucleosides). As also noted above, examples of modified nucleic acid bases are summarized by Limbach et al., (1994, Nucleic Acids Res. 22, 2183-2196).

In particular illustrative embodiments, the oligomer of the invention comprises one or more modified nucleosides. In certain embodiments, all the nucleosides are modified, and in other certain embodiments, none of the nucleosides are modified. In various embodiments, the oligomer comprises about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or 100% modified nucleosides.

As used herein, the terms “modified base,” “modified nucleic acid base,” and “modified nucleobase,” are used interchangeably herein and refer to bases other than adenine, guanine, cytosine, thymine, and uracil at the 1′ position or their equivalents; such bases can be used at any position, for example, within the catalytic core of an enzymatic nucleic acid molecule and/or in the substrate-binding regions of the nucleic acid molecule.

Exemplary chemically modified and other natural nucleic acid bases that can be introduced into nucleic acids include, but are not limited to, 5-methylcytosine (5-me-C), 5-hydroxymethyl cytosine, xanthine, hypoxanthine, inosine, dihydrouridine, hypoxathanine, isocytosine, isoguanine, N6-methyladenosine, pseudouracil, pyridin-2-one, pyridin-4-one, quesosine, threonine derivatives, uridine-5-oxyacetic acid, wybutosine, wybutoxosine, xathanine, β-D-galactosylqueosine, β-D-mannosylqueosine, 2,2-dimethylguanosine, 15-halocytosine, 15-halouracil, 1-methyladenosine, 1-methylinosine, 2-aminoadenine, 2-methyladenosine, 2-methylguanosine, 2-methylthio-N6-isopentenyladenosine, 2-propyl adenine, 2-propyl guanine, 2-thiocytidine, 2-thiocytosine, 2-thiothymine, 2-thiouracil, 2-thiouridine, 3-deazaadenine or the like, 3-deazaguanine, 3-methyl uracil, 3-methylcytidine, 3-nitropyrrole, 4-acetyltidine, 4-thiouracil, 4-thiouridine, 5-(carboxyhydroxymethyl)uridine, 5-carboxymethylaminomethyl-2-thiouridine, 5-alkylcytidines (e.g., 5-methylcytidine), 5-alkyluridines (e.g., ribothymidine), 5-carboxymethylaminomethyluridine, 5-halo substituted uracil or cytosine, 5-halouridine (e.g., 5-bromouridine) or 6-azapyrimidines or 6-alkylpyrimidines (e.g., 6-methyluridine), 5-hydroxymethyl cytosine, 5-methoxyaminomethyl-2-thiouridine, 5-methyl-2-thiouridine, 5-methylaminomethyluridine, 5-methylcarbonyhnethyluridine, 5-methylcytosine, 5-methyloxyuridine, 5-nitroindole, 5-propynyl cytosine, 5-propynyl uracil, 5-uracil, 6-azo cytosine, 6-azo thymine, 6-azo uracil, 6-methyl adenine, 6-methyl guanine, 7-deazaadenine, 7-deazaguanine, 7-methyladenine, 7-methylguanine, 7-methylguanosine, 8-amino adenine or guanine, 8-azaadenine, 8-azaguanine, 8-halo adenine or guanine, 8-hydroxyl adenine or guanine, 8-thioalkyl adenine or guanine, 8-thiol adenine or guanine or the like.

Other exemplary modified nucleobases that can be used in the methods and compositions of the present invention, include, without limitation, modified nucleobases include tricyclic pyrimidines such as phenoxazine cytidine (1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified nucleobases may also include those in which the purine or pyrimidine base is replaced with other heterocycles, for example 7-deazaadenine, 7-deazaguanosine, 2-aminopyridine and 2-pyridone.

Further illustrative examples of nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. In certain embodiments, particular nucleobases are useful for increasing the binding affinity of the oligomers of the invention, e.g., 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications.

In particular illustrative embodiments, polycyclic or heterocyclic compounds can be used as nucleobases in the oligomers of the present invention. Exemplary U.S. patents that disclose these compounds include, but are not limited to, U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; 6,007,992; and 5,681,941, and U.S. Patent Application Publication 20030158403, U.S. Pat. No. 6,028,183, each of which is incorporated herein by reference in its entirety.

In particular illustrative embodiments, the oligomer of the invention comprises one or more modified nucleobases. In certain embodiments, all the nucleobases are modified, and in other certain embodiments, none of the nucleobases are modified. In various embodiments, the oligomer comprises about 5%, about 10′)/0, about 15%, about 20%, about 25%, about 30%, about 35%, about 40%, about 45%, about 50%, about 55%, about 60%, about 65%, about 70%, about 75%, about 80%, about 85%, about 90%, about 95% or 100% modified nucleobases.

In one embodiment, oligomers of the present invention comprise one or more stabilizing groups that are generally attached to one or both termini of oligomers to enhance properties such as, for example, nuclease stability. Exemplary stabilizing groups include cap structures. As used herein, the terms “cap structure” or “terminal cap moiety” refer to a chemical modification, which has been incorporated at either or both ends of oligonucleotide. These terminal modifications can protect the oligomers having terminal nucleic acid molecules from exonuclease degradation, and can help in delivery and/or localization within a cell. The cap can be present at the 5′-terminus (5′-cap) or at the 3′-terminus (3′-cap) or can be present on both termini.

In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

In a further non-limiting example, 3′-cap structures of the present invention include, 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties.

2. Modified Sugars

The present invention contemplates, in part, that oligomers can comprise one or more modified or non-modified nucleosides and nucleotides comprising modified sugars and/or with substituent groups. Oligomers of the present invention comprising one or more sugar modifications or substituents can have increased resistance to nucleases, increased binding affinity to a target nucleic acid and other beneficial biological properties.

In particular illustrative embodiments, modified sugars include but are not limited to carbocyclic or acyclic sugars; sugars having substituent groups at one or more of their 2′, 3′, or 4′ positions; sugars having substituents in place of one or more hydrogen atoms of the sugar; and sugars having a linkage between any two other atoms in the sugar.

In preferred embodiments, oligomers of the present invention comprise one or more sugars modified at the 2′ position or those which have a bridge between any two atoms of the sugar (such that the sugar is bicyclic). In particular illustrative embodiments, sugar modifications or substituents suitable for use in oligomers of the present invention include, but are not limited to compounds comprising a sugar substituent group selected from: OH; F; O-, S-, or N-alkyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary modifications include but are not limited to OH, halogen, O-alkyl, S-alkyl, N-alkyl, O-alkenyl, S-alkenyl, N-alkenyl, O-alkynyl, S-alkynyl, N-alkynyl, O-alkyl-O-alkyl, alkaryl, aralkyl, O-alkaryl, O-aralkyl, SH, SCH₃, OCN, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, alkoxyalkoxy, dimethylaminooxyethoxy, allyl, and O-allyl, wherein the alkyl, alkenyl and alkynyl can be substituted or unsubstituted C₁ to C₁₀ alkyl or C₂ to C₁₀ alkenyl and alkynyl.

In certain illustrative embodiments, the preferred sugar modification is selected from the group consisting of: 2-methoxyethoxy (also known as 2′-O-methoxyethyl, 2′-MOE, or 2′-OCH₂CH₂OCH₃), 2′-O-methyl(2′-O—CH₃), 2′-fluoro (2′-F), or bicyclic sugar modified nucleosides having a bridging group connecting the 4′ carbon atom to the 2′ carbon atom wherein example bridge groups include —CH₂—O—, —(CH₂)₂—O— or —CH₂—N(R)—O wherein R is H or C₁-C₁₂ alkyl.

2′-MOE modified sugars provide increased nuclease resistance and a very high binding affinity to nucleotides/nucleosides. Increased binding affinity of the 2′-MOE substitution can be greater than many similar 2′ modifications such as O-methyl, O-propyl, and O-aminopropyl. Oligomers comprising one or a plurality of 2′-MOE modifications or substituents have also been used as in vivo antisense inhibitors of gene expression (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

2′-Sugar substituent groups may be in the arabino (up) position or ribo (down) position. One 2′-arabino modification is 2′-F. Similar modifications can also be made at other positions on the oligomer, particularly the 3′ position of the sugar on the 3′ terminal nucleoside or in 2′-5′ linked oligonucleotides and the 5′ position of 5′ terminal nucleotide. Oligomers may also have sugar mimetics such as cyclobutyl moieties in place of the pentofuranosyl sugar. Illustrative U.S. patents that describe the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, each of which is herein incorporated by reference in its entirety.

In other particular embodiments, the present invention contemplates, in part, sugar modifications that are bicyclic thereby locking the sugar conformational geometry. Bicyclic sugar modifications impart increased resistance to nucleases, increased binding affinity to target nucleic acids, and other beneficial biological property to the oligomers contemplated herein.

In one illustrative embodiment, the modified bicyclic sugar moiety comprises a 4′-CH₂—O-2′ bridge, e.g., locked nucleic acids. As used herein, the term “locked nucleic acids” (LNAs), refers to conformationally restricted oligonucleotide analogues containing a methylene bridge that connects the 2′-O of ribose with the 4′-C (see, Singh et al., Chem. Commun., 1998, 4:455-456). LNA oligonucleotides contain one or more nucleotide building blocks in which an extra methylene bridge, as noted above, fixes the ribose moiety either in the C3′-endo (β-D-LNA) or C2′-endo (α-L-LNA) conformation.

LNA and LNA analogues display very high duplex thermal stabilities with complementary DNA and RNA, stability towards 3′-exonuclease degradation, and good solubility properties. Synthesis of the LNA analogues of adenine, cytosine, guanine, 5-methylcytosine, thymine and uracil, their oligomerization, and nucleic acid recognition properties have been described (see Koshkin et al., Tetrahedron, 1998, 54:3607-3630). Studies of mismatched sequences show that LNA obey the Watson-Crick base pairing rules with generally improved selectivity compared to the corresponding unmodified reference strands. Antisense oligonucleotides containing LNAs have been described (Wahlestedt et al., Proc. Natl. Acad. Sci. U.S.A., 2000, 97:5633-5638), which were efficacious and non-toxic. In addition, the LNA/DNA copolymers were not degraded readily in blood serum and cell extracts.

LNAs form duplexes with complementary DNA or RNA or with complementary LNA, with high thermal affinities. The universality of LNA-mediated hybridization has been emphasized by the formation of exceedingly stable LNA:LNA duplexes (Koshkin et al., J. Am. Chem. Soc., 1998, 120:13252-13253). LNA:LNA hybridization was shown to be the most thermally stable nucleic acid type duplex system, and the RNA-mimicking character of LNA was established at the duplex level. Introduction of three LNA monomers (T or A) resulted in significantly increased melting points toward DNA complements.

Synthesis of 2′-amino-LNA (Singh et al., J. Org. Chem., 1998, 63, 10035-10039) and 2′-methylamino-LNA has been described and thermal stability of their duplexes with complementary RNA and DNA strands reported. Preparation of phosphorothioate-LNA and 2′-thio-LNA have also been described (Kumar et al., Bioorg. Med. Chem. Lett., 1998, 8:2219-2222).

Other modified bicyclic sugar moieties include xylo sugar analogs (see, e.g., U.S. Patent Application Publication No.: 2003/0082807). Further illustrative embodiments of bicyclic sugar modified nucleosides include, but are not limited to, 4′-(CH₂)₂—O-2′ (U.S. Patent Application Publication Nos.: US 2002/0147332 and 2003/0207841; U.S. Pat. Nos. 6,268,490 and 6,670,461).

In particular illustrative embodiments, each nucleobase of the oligomer of the invention comprises a modified or substituted sugar. In certain embodiments, the oligomer comprises nucleotides/nucleosides having about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or 100% modified or substituted sugars.

In one illustrative embodiment, an anti-miRNA molecule of the present invention comprises one or more nucleotides/nucleosides that have a modified sugar or substituent selected from the group consisting of 2′-MOE, 4′-CH₂—O-2′, and 4′-(CH₂)₂—O-2′. An anti-miRNA molecule can comprise the same modified sugar or the substituent throughout, or a combination of modified sugars or substituents, without limitation.

3. Internucleotide/Internucleoside Linkages

The present invention contemplates, in part, that oligomers can comprise one or more modified internucleotide and/or internucleoside linkages. Oligomers of the present invention comprising one or more modified internucleotide and/or internucleoside linkages can have increased resistance to nucleases, increased binding affinity to a target nucleic acid and other beneficial biological properties. As used herein, the term “modified internucleotide linkage” refers to a linkage between nucleosides comprising a phosphorus atom in their internucleoside backbone. As used herein, the term “modified internucleoside linkage” refers to a linkage between nucleosides that lacks a phosphorus atom in their internucleoside backbone.

In particular illustrative embodiments, 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or 100% of the internucleotide and/or internucleoside linkages may be modified as discussed herein.

In certain illustrative embodiments, oligomers comprise modified, i.e., non-naturally occurring internucleoside linkages that are often selected over naturally occurring forms because of desirable properties such as, for example, enhanced cellular uptake, enhanced affinity for other oligonucleotides or nucleic acid targets and increased resistance to nucleases.

In one illustrative embodiment, an oligomer comprises one or more phosphorothioate internucleotide linkages. Other illustrative modified internucleotide linkages include, for example, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage.

Illustrative U.S. patents that teach the preparation of internucleotide linkages include, but are not limited to, U.S. Pat. Nos. 3,687,808; 4,469,863; 4,476,301; 5,023,243, 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 5,625,050, 5,489,677, and 5,602,240 each of which is herein incorporated by reference in its entirety.

In particular illustrative embodiments, an oligomer of the present invent comprises one or more modified internucleoside linkages that do not include a phosphorus atom.

Exemplary modified internucleoside linkages that do not include a phosphorus atom therein have backbones include short chain alkyl or cycloalkyl internucleoside linkages, mixed heteroatom and alkyl or cycloalkyl internucleoside linkages, or one or more short chain heteroatomic or heterocyclic internucleoside linkages. Additional modified internucleoside linkages include morpholino linkages (formed in part from the sugar portion of a nucleoside); siloxane linkages; sulfide, sulfoxide and sulfone linkages; formacetyl and thioformacetyl linkages; methylene formacetyl and thioformacetyl linkages; riboacetyl linkages; alkene containing linkages; sulfanate linkages; methyleneimino and methylenehydrazino linkages; sulfonate and sulfonamide linkages; amide linkages; and others having mixed N, O, S and CH₂ component parts.

Illustrative U.S. patents that teach the preparation of the above non-phosphorous-containing oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, each of which is herein incorporated by reference in its entirety.

In one embodiment, an anti-miRNA of the present invention comprises one or more modified internucleotide/internucleoside linkages selected from the group consisting of: a phosphorothioate, phosphorodithioate, phosphotriester, aminoalkylphosphotriester, alkyl phosphonates, phosphinate, phosphoramidate, thionophosphoramidate, thionoalkylphosphonate, thionoalkylphosphotriester, selenophosphate, boranophosphate, morpholino, siloxane, sulfide, sulfoxide, sulfone, formacetyl, thioformacetyl, methylene formacetyl, riboacetyl, alkene-containing backbone, sulfamate, methyleneimino, methylenehydrazino, sulfonate, sulfonamide, or amide.

In a preferred embodiment, the modified internucleotide linkage is a phosphorothioate linkage.

4. Mimetics

Oligomers can also include oligonucleotide mimetics. As used herein, the term “mimetic” refers to oligomers wherein only the furanose ring or both the furanose ring and the internucleotide linkage are replaced with novel groups, replacement of only the furanose ring with for example a morpholino ring, is also referred to in the art as being a sugar surrogate. The heterocyclic base moiety or a modified heterocyclic base moiety is maintained for hybridization with an appropriate target nucleic acid.

Oligonucleotide mimetics can include oligomers such as peptide nucleic acids (PNA) and cyclohexenyl nucleic acids (known as CeNA, see Wang et al., J. Am. Chem. Soc., 2000, 122, 8595-8602)

Illustrative U.S. patents that teach the preparation of oligonucleotide mimetics include, but are not limited to, U.S. Pat. Nos. 5,539,082; 5,714,331; and 5,719,262, each of which is herein incorporated by reference in its entirety.

Another class of oligonucleotide mimetic is referred to as phosphonomonoester nucleic acid and incorporates a phosphorus group in the backbone. This class of oligonucleotide mimetic is reported to have useful physical and biological and pharmacological properties in the areas of inhibiting gene expression (antisense oligonucleotides, ribozymes, sense oligonucleotides and triplex-forming oligonucleotides). Another oligonucleotide mimetic has been reported wherein the furanosyl ring has been replaced by a cyclobutyl moiety.

5. Conjugates

The present invention contemplates, in part, oligomers comprising at least one MGB and one or a plurality of covalently linked conjugate moieties. As used herein, the term “conjugate moiety” refers to lipophilic molecules including, but not limited to lipophilic moieties such as, cholesterols, lipids, phospholipids, folates, polyamines, polyamides, polyethylene glycols, polyethers, groups that enhance the pharmacodynamic properties of oligomers, and groups that enhance the pharmacokinetic properties of oligomers.

A conjugate moiety may be covalently linked to one or more oligomeric units of an oligomer via a linker, as described elsewhere herein or, directly covalently linked to one or more oligomeric units of the oligomer via a functional group, e.g., primary or secondary hydroxyl groups. In various embodiments, one or more of the same or different conjugate moieties can be covalently linked to an oligomer. The conjugate moiety can be conjugated to the 5′ end and/or 3′ end of oligomer, and/or to an internal oligomeric unit of an oligomer. In preferred embodiments, a conjugate moiety is conjugated to one or more bases at the 3′ end of the oligomer.

In particular illustrative embodiments conjugate moieties suitable for use in oligomers of the present invention include, but are not limited to lipid moieties such as a cholesterol moiety (Letsinger et al., Proc. Natl. Acad. Sci. USA, 1989, 86, 6553-6556), cholic acid (Manoharan et al., Bioorg. Afied Chem. Let., 1994, 4, 1053-1060), a thioether, e.g., hexyl-5-tritylthiol (Manoharan et al., Ann. N.Y. Acad. Sci., 1992, 660, 306-309; Manoharan et al., Bioorg. Med Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937.

In certain illustrative embodiments, the oligomers are conjugated a lipophilic moiety.

Exemplary lipophilic moieties include, but are not limited to a lipid, cholesterol, oleyl, retinyl, cholesteryl residues, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine.

In one embodiment, the lipophilic moiety is cholesterol.

6. Minor Groove Binding Moieties

The present invention contemplates, in part, an oligomer comprising one or a plurality of the same or different minor groove binding moieties (MGBs) conjugated to oligomer, as described elsewhere herein. A variety of suitable minor groove binders have been described in the literature. See, e.g., Wemmer, D. E., and Dervan P. B., Current Opinion in Structural Biology, 7:355 361 (1997); Walker, W. L., Kopka, J. L. and Goodsell, D. S., Biopolymers, 44:323 334 (1997); Zimmer, C & Wahnert, U. Prog. Biophys. Molec. Bio. 47:31 112 (1986); and Reddy, B. S. P., Dondhi, S. M., and Lown, J. W., Pharmacol. Therap., 84:1 111 (1999). Without wishing to be bound to any particular theory, an oligomer conjugated to one or more MGBs substantially increases the activity, cellular distribution, tissue distribution, and/or organ distribution, or cellular uptake, tissue uptake, or organ uptake of the oligonucleotide, hybridization affinity to a target nucleic acid, hybridization specificity to a target nucleic acid, cellular uptake, resistance to nucleases, and substantially decreases therapeutic toxicity compared to an oligomer that is not conjugated or linked to an MGB.

As used herein, the phrase “groups that enhance pharmacodynamic properties” refers to MGBs that improve the nuclear entry, and/or increases the strength and specificity of sequence-specific hybridization between the oligomer and target nucleic acid, e.g., mRNA, pri-miRNA. As used herein, the phrase “groups that enhance pharmacokinetic properties” refers to MGBs that improve oligomer uptake in cell, tissue, and/or organ distribution, in desired subcellular compartments, metabolism, or excretion.

MGBs can be conjugated to the 5′-end and/or 3′-end of an oligomer, and/or to one or more internal oligomer units of the oligomer, so long as the desired function of the oligomer is not compromised. In one embodiment, as used herein, the term “desired function” refers to an oligomer's ability to decrease the expression of one or more RNAs in a plurality of cell types, tissues, or organs, e.g., 1, 2, 3, 4, 5, 6, 7, 8, 9, 10, or more.

In a particular embodiment, an oligomer contemplated by the present invention comprises an MGB conjugated to the 5′-end of the oligomer, an MGB conjugated to the 3′-end of the oligomer, and one or more MGBs conjugated to an internal unit of the oligomer. In certain embodiments, 1, 2, 3, 4, 5, 6, 7, 8, 9, 10, or more MGBs may be conjugated to an oligomer. In another embodiment, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the oligomeric units of the oligomer are conjugated to an MGB.

In one preferred embodiment, an MGB is conjugated to the 5′-end of the oligomer. In another preferred embodiment, an MGB is conjugated to the 3′-end of the oligomer. In yet another preferred embodiment, an MGB is conjugated to the 5′-end of the oligomer and the same type or a different type of MGB is conjugated to the 3′-end of the oligomer.

In particular illustrative embodiments, MGBs that can be conjugated to an oligomer include, but are not limited to, naturally occurring compounds such as netropsin, distamycin and lexitropsin, mithramycin, chromomycin A3, olivomycin, anthramycin, sibiromycin, as well as further related antibiotics and synthetic derivatives. Certain bisquarternary ammonium heterocyclic compounds, diarylamidines such as pentamidine, stilbamidine and berenil, CC-1065 and related pyrroloindole and indole polypeptides, Hoechst 33258, 4′-6-diamidino-2-phenylindole (DAPI) as well as a number of oligopeptides consisting of naturally occurring or synthetic amino acids are minor groove binder compounds.

In preferred illustrative embodiments, MGBs that can be conjugated to an oligomer are selected from the group consisting of: netropsin, distamycin and lexitropsin, mithramycin, chromomycin A₃, olivomycin, anthramycin, and sibiromycin.

In particular preferred embodiments, MGB moieties include multimers of 1,2-dihydro-(3H)-pyrrolo[3,2-e]indole-7-carboxylate (DPI), N-3-carbamoyl 1,2-dihydro-(3H)-pyrrolo[3,2-e]indole-7-carboxylate (CDPI) and multimers of N-methylpyrrole-4-carbox-2-amide (MPC). Particularly preferred MGB moieties comprise DPI₍₁₋₁₀₎, CDPI₍₁₋₁₀₎, and MPC₍₁₋₁₀₎. In a more preferred embodiment, the MGB comprises CDPI₍₁₋₃₎ or MPC₍₁₋₃₎. In a certain preferred embodiment, the MGB comprises CDPI₃ or CDPI₄.

Representative U.S. patents that describe MGBs include, but are not limited to, U.S. Pat. Nos. 5,801,155; 6,084,102; 6,312,894; 6,492,346; and 7,205,105 each of which is herein incorporated by reference in its entirety.

7. Linkers

The present invention contemplates, in part, an oligomer comprising one or a plurality of the same or different functional moieties, e.g., MGBs, cholesterol, conjugated to an oligomer. Functional moieties can be covalently attached to various positions of an oligomer directly or via a linker or linking group. As used herein, the terms “linker” and “linking group” and equivalents thereof, refers to a molecule that is used to covalently attach portions of the oligomer to a functional moiety, e.g., MGBs, cholesterol. Generally, a linking group can include linear or acyclic portions, cyclic portions, aromatic rings or combinations thereof.

In one particular illustrative embodiment, a linking group can have about 1 to about 200 main chain atoms, about 1 to about 100 main chain atoms, about 1 to about 50 main chain atoms, about 1 to about 30 main chain atoms, or about 1 to about 15 main chain atoms. As used herein, the term “main chain atom” refers to only those atoms between the oligomer and the functional moiety that are joined in a continuous line, including all ring atoms, but not including any pendant atoms or groups. In one embodiment, main chain atoms are selected from the group consisting of: C, O, N, S, P and Si.

In one illustrative embodiment, a linking group can be a bivalent or trivalent linker having from about 3 to 100 main chain atoms, selected from C, O, N, S, P, and Si. In other illustrative embodiments, a linker comprises a branched aliphatic chain, a heteroalkyl chain, one or more substituted ring structures, or combinations thereof. Linkers generally have functional groups to accomplish conjugation. Illustrative examples of functional groups used to covalently bind molecules to the linker include, but are not limited to: hydroxyl, amino, alkoxy, carboxy, benzyl, phenyl, nitro, thiol, thioalkoxy, phosphates, phosphonium ions, halogen, alkyl, aryl, alkenyl, and alkynyl groups.

In preferred illustrative embodiments, a linking group comprises a formula selected from the group consisting of: —O(CH₂)₆NH—, —P(═O)(OH)O(CH₂)₆NH—; —OCH₂CH(OH)CH₂NHCOCH₂CH₂NH—, —HN(CH₂)₅CO—; —P(═O)(OH)O(CH₂)₄NH—; —P(═O)(OH)(OCH₂CH₂)₆OP(═O)(OH)O(CH₂)₆NH—; —(CH₂)₅OP(═O)(OH)—, and hydroxy{[5-(hydroxymethyl)-1-methylpyrrolidin-3 yl]oxy}oxophosphonium.

In particular preferred embodiments, a linking group comprises a formula selected from the group consisting of: hydroxy{[5-(hydroxymethyl)-1-methylpyrrolidin-3yl]oxy}oxophosphonium; —P(═O)(OH)O(CH₂)₄NH—; —P(═O)(OH)(OCH₂CH₂)₆OP(═O)(OH)O(CH₂)₆NH—; and —P(═O)(OH)O(CH₂)₆NH—.

In certain preferred embodiments, a linking group comprises the formula —P(═O)(OH)O(CH₂)₆NH—.

Illustrative U.S. patents that describe suitable methods for attaching MGBs and other functional moieties through linkers to oligomers include, but are not limited to, U.S. Pat. Nos. 5,512,677; 5,419,966; 5,696,251; 5,585,481; 5,801,155; 5,942,610; and 5,736,626.

### J. Compositions and Formulations

The oligomers used in accordance with the present invention can be conveniently and routinely made through the well-known in-vitro technique of solid phase oligonucleotide synthesis. Equipment for such synthesis is sold by several vendors including, for example, Applied Biosystems (Foster City, Calif.). Any other means for such synthesis known in the art may additionally or alternatively be employed.

As described in detail below, compositions of the present invention comprising an oligomer, can be specially formulated, for example by admixing, encapsulating, conjugating or otherwise associating with other molecules, molecule structures or mixtures of compounds, as for example, liposomes, emulsions, microemulsions, and receptor targeted molecules. The formulated compositions are suitable for administration to a subject in need of treatment in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, systemic (e.g., intravenous, intraarterial, intravascular), or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally, as an inhalent or aerosol.

As used herein, the term “effective amount” refers to an amount of an oligomer that is effective at dosages and for periods of time necessary, to achieve the desired therapeutic or prophylactic result. Effective amounts include therapeutically effective amounts and prophylactically effective (preventative) amounts. An effective amount may vary according to factors such as the disease state, age, sex, and weight of the individual, and the ability of the one or more repressors and/or activators to elicit a desired response in the individual.

A “therapeutically effective amount” of an oligomer, as disclosed elsewhere is also one in which any toxic or detrimental effects of an oligomer are outweighed by the therapeutically beneficial effects. The term “therapeutically effective amount” includes an amount that is effective to reduce, inhibit, prevent, or treat a disease, disorder, or condition associated with increased activity and/or RNA expression in one or more cells, tissues, or organs in a mammal (e.g., a subject in need of treatment). For example, a therapeutically effective amount of an oligomer, can be an amount sufficient to cause a 5%, 10%, 15%, 20%, 25%, 30%, 35%, 40%, 45%, 50%, 55%, 60%, 65%, 70%, 75%, 80%, 90%, 95%, or 100% improvement in organ function or physiological indicators of amelioration of disease symptoms (e.g., liver function, lung function, kidney function) relative to organ function observed prior to administration of the oligomer. In particular embodiments, wherein the subject being treated has cancer, a decrease in tumor volume, metastases, or circulating cancer cells would be suitable to indicate a therapeutically effective amount.

A “prophylactically effective amount” refers to an amount of an oligomer that is effective at dosages and for periods of time necessary, to achieve the desired prophylactic result. Typically, but not necessarily, since a prophylactic dose is used in subjects prior to or at an earlier stage of disease, the prophylactically effective amount is less than the therapeutically effective amount.

As used herein, the term, “pharmaceutically-acceptable carrier” refers to a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not adversely affecting the subject being treated. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) a pharmaceutically acceptable cell culture medium; and (23) other non-toxic compatible substances employed in pharmaceutical formulations.

The preparation of an aqueous composition that contains an oligomer of the invention is also contemplated. Typically, such compositions are prepared as injectables, either as liquid solutions or suspensions; solid forms suitable for solution in, or suspension in, liquid prior to injection can also be prepared.

Certain embodiments include “pharmaceutically-acceptable salts,” including hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al., J. Pharm. Sci. 1977; 66:1-19). Additional examples include base addition salts such as the hydroxide, carbonate or bicarbonate of a pharmaceutically-acceptable metal cation, with ammonia, or with a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra).

In another embodiment, the amount of active ingredient (e.g., an oligomer) in a single dosage from that is required to produce a therapeutic effect is about 0.1% active ingredient, about 1% active ingredient, about 5% active ingredient, about 10% active ingredient, about 15% active ingredient, about 20% active ingredient, about 25% active ingredient, about 30% active ingredient, about 35% active ingredient, about 40% active ingredient, about 45% active ingredient, about 50% active ingredient, about 55% active ingredient, about 60% active ingredient, about 65% active ingredient, about 70% active ingredient, about 75% active ingredient, about 80% active ingredient, about 85% active ingredient, about 90% active ingredient, or about 95% active ingredient or more, including all ranges of such values.

In certain embodiments, a composition of the present invention comprises an excipient selected from the group consisting of cyclodextrins and derivatives, celluloses, liposomes, emulsions, microemulsions, micelle forming agents, e.g., bile acids, and polymeric carriers, e.g., polyesters and polyanhydrides; and a compound of the present invention. In certain embodiments, an aforementioned composition renders orally bioavailable an oligomer of the present invention.

For administration by inhalation, an oligomer for use according to the present invention can be conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

Compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A composition of the present invention may also be administered as a bolus, electuary or paste.

In solid dosage forms, for compositions of the invention suitable for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Compositions of the present invention which are suitable for vaginal administration also include pessaries, tampons, creams, gels, pastes, foams or spray formulations containing such carriers as are known in the art to be appropriate.

Dosage forms for the topical or transdermal administration of a composition as provided herein include powders, sprays, ointments, pastes, creams, lotions, gels, solutions, patches and inhalants. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a composition of the present invention to the body. Absorption enhancers can also be used to increase the flux of the agent across the skin.

Ophthalmic formulations, eye ointments, powders, solutions and the like, are also contemplated as being within the scope of this invention.

Compositions of this invention suitable for parenteral administration comprise pharmaceutically-acceptable sterile isotonic aqueous or nonaqueous solutions, dispersions, liposomes, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Injectable depot forms are made by forming microencapsule matrices of the subject compounds in biodegradable polymers such as polylactide-polyglycolide. Examples of other biodegradable polymers include poly-(orthoesters) and poly-(anhydrides).

In certain embodiments, microemulsification technology may be utilized to improve bioavailability of hydrophobic (water insoluble) anti-miRNA molecules (Dordunoo et al., Drug Development and Industrial Pharmacy. 1991; 17(12), 1685-1713 and REV 5901 (Sheen, P. C, et al., J Pharm Sci 80(7), 712-714, 1991).

As used herein, the phrases “parenteral administration” and “administered parenterally” refer to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

As used herein, the terms “systemic administration,” “administered systemically,” “peripheral administration” and “administered peripherally” refer to the administration of a compound, drug or other material other than directly into the central nervous system, such that it enters the patient's system and, thus, is subject to metabolism and other like processes, for example, subcutaneous administration.

In general, a suitable daily dose of a composition comprising an oligomer as described herein, will be that amount of the oligomer which is the lowest dose effective to produce a therapeutic effect. Administration of an oligomer can be performed in a single composition or multiple compositions, separately or at the same time. Several unit dosage forms may be administered at about the same time. A dose employed may be determined by a physician or qualified medical professional, and depends upon the desired therapeutic effect, the route of administration and the duration of the treatment, and the condition of the patient.

The term “dose” includes, but is not limited to an effective dose, such as, for example, an acute dose, a sub-acute dose, and a chronic or continuous dose.

The terms “acute dose” or “acute administration” of one or more active agents mean the scheduled administration of the active agent(s) to a patient on an as-needed basis at a dosage level determined by the attending physician to elicit a relatively immediate desired reaction in the patient, given the patient's age and general state of health.

A “sub-acute dose” is a dose of the active agent(s) at a lower level than that determined by the attending physician to be required for an acute dose, as described above. Sub-acute doses may be administered to the patient on an as-needed basis, or in a chronic, or on-going dosing regimen.

The terms “chronic dose” or “continuous administration” of the active agent(s) mean the scheduled administration of the active agent(s) to the patient on an on-going day-to-day basis.

An effective dose will generally depend upon the factors described above. Generally, oral, nasal, intravenous, intracerebroventricular, subcutaneous, and inhalation doses of an oligomer for a subject, will range from about 0.000001 to about 1000 mg per kilogram, about 0.000005 to about 950 mg per kilogram, about 0.00001 to about 850 mg per kilogram, about 0.00005 to about 750 mg per kilogram, about 0.0001 to about 500 mg per kilogram, about 0.0005 to about 250 mg per kilogram, about 0.001 to about 100 mg per kilogram, about 0.001 to about 50 mg per kilogram, about 0.001 to about 25 mg per kilogram, about 0.001 to about 10 mg per kilogram, about 0.001 to about 1 mg per kilogram, about 0.005 to about 100 mg per kilogram, about 0.005 to about 50 mg per kilogram, about 0.005 to about 25 mg per kilogram, about 0.005 to about 10 mg per kilogram, about 0.005 to about 1 mg per kilogram, about 0.01 to about 100 mg per kilogram, about 0.01 to about 500 mg per kilogram, about 0.01 to about 250 mg per kilogram, about 0.01 to about 100 mg per kilogram, about 1 to about 100 mg per kilogram, about 1 to about 50 mg per kilogram, about 1 to about 25 mg per kilogram, about 1 to about 20 mg per kilogram, about 10 to about 100 mg per kilogram, about 10 to about 50 mg per kilogram, about 10 to about 25 mg per kilogram of body weight per day; or about 10 mg, about 20 mg, about 30 mg, about 40 mg, about 50 mg, about 60 mg, about 70 mg, about 80 mg, about 90 mg, or about 100 mg per kilogram of body weight per day.

In another embodiment, an oligomer is administered intravenously, orally, by inhalation, nasally, or parenterally to a subject at a dose of about 0.25 to 3 g per kg, about 0.5 to 2.5 g per kg, about 1 to 2g per kg, about 1.25 to 1.75 g per kg or about 1.5 g per kg of body weight per day.

In particular embodiments, an oligomer is administered orally, by inhalation, nasally, or parenterally (e.g., intravenously) to a subject at a dose of about 10 g per kg, about 0.25 g per kg, about 0.50 g per kg, about 0.75 g per kg, about 1.0 g per kg, about 1.25 g per kg, about 1.50 g per kg, about 1.75 g per kg, or about 2.00 g per kg of body weight per day.

In other related embodiments, an oligomer is administered orally, by inhalation, nasally, or parenterally (e.g., intravenously) to a subject at a dose of about 0.01 μg to 1 mg per kg, about 0.1 to 100 μg per kg, or about 1 to 10 μg per kg or any increment of concentration in between. For example, in particular embodiments, an oligomer is administered orally nasally, or parenterally to a subject at a dose of about 1 μg per kg, about 2 μg per kg, about 3 μg per kg, about 4 μg per kg, about 5 μg per kg, about 6 μg per kg, about 7 μg per kg, about 8 μg per kg, about 9 μg per kg, or about 10 μg per kg.

In particular embodiments, an oligomer is administered orally, by inhalation, nasally, or parenterally (e.g., intravenously) to a subject at a dose of about 0.005 μg per kg, about 0.01 μg per kg, about 1.0 μg per kg, about 10 μg per kg, about 50 μg per kg, about 100 μg per kg, about 250 μg per kg, about 500 μg per kg, or about 1000 μg per kg

A composition may be administered 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Moreover, multiple administrations of the same or different compositions of the present invention may be administered, multiples times, for extended periods of time, as noted above.

In particular embodiments, the frequency of delivery of a composition is once a day, twice a day, three times day, four times a day, once every two days, or once a week or any intervening frequency.

In particular embodiments, the duration of continuous delivery of a composition is between 30 seconds and 24 hours, between 30 seconds and 12 hours, between 30 seconds and 8 hours, between 30 seconds and 6 hours, between 30 seconds and 4 hours, between 30 seconds and 2 hours, between 30 seconds and 1 hour, between 30 seconds and 30 minutes, between 30 seconds and 15 minutes, between 30 seconds and 10 minutes, between 30 seconds and 5 minutes, between 30 seconds and 2 minutes, between 30 seconds and 1 minute or any intervening period of time.

Additional methods of formulating compositions known to the skilled artisan, for example, as described in the Physicians Desk Reference, 62nd edition. Oradell, N.J.: Medical Economics Co., 2008; Goodman & Gilman's The Pharmacological Basis of Therapeutics, Eleventh Edition. McGraw-Hill, 2005; Remington: The Science and Practice of Pharmacy, 20th Edition. Baltimore, Md.: Lippincott Williams & Wilkins, 2000; and The Merck Index, Fourteenth Edition. Whitehouse Station, N.J.: Merck Research Laboratories, 2006; are hereby incorporated by reference in relevant parts

### K. Methods of Use

The present invention generally provides improved oligomers that are safer, more efficient, and more potent than existing therapies. The present invention further provides oligomers that have surprising and unexpected increased biodistribution to cells, tissues, and/or organs. Accordingly, use of the oligomers of the invention to treat diseases, disorders and conditions characterized or associated with increased gene expression and increased miRNA expression and/or activity are provided.

1. Methods of Inhibiting RNA Expression and/or Activity

In a particular illustrative embodiment, the present invention provides, in part, a method to inhibit the expression and/or activity of an RNA comprising contacting a target RNA with an oligomer that hybridizes to the target. Without wishing to be bound to any particular theory, the present invention contemplates, in part, that hybridizing an oligomer to a target RNA sequence can inhibit the expression and/or activity of the RNA.

2. Methods of Treatment

As noted above, oligomers of the present invention are useful in a number of clinical settings, such as for example, where a patient is suffering from a condition or disease characterized by increased expression of one or more particular RNAs. The oligomers of the invention are particularly useful in clinical setting where a patient is in need of treatment for a condition or disease characterized by increased expression of one or more RNAs expressed in different cells, tissues, and/or organs.

In various embodiments, the present invention contemplates, in part, a method of treating a subject having a systemic disease, disorder or condition that is characterized by increased activity of one or more RNAs in one or more cells, tissues, and/or organs.

The term “subject” as used herein includes, but is not limited to, an organism; a mammal, including, e.g., a human, non-human primate (e.g., baboon, orangutan, monkey), mouse, pig, cow, goat, dog, cat, rabbit, rat, guinea pig, hamster, horse, monkey, sheep, or other non-human mammal; a non-mammal, including, e.g., a non-mammalian vertebrate, such as a bird (e.g., a chicken or duck) or a fish, and a non-mammalian invertebrate. In preferred embodiments, the subject is human.

Typical subjects include those that have or are at risk for having tumor mediated angiogenesis, cancer, inflammation, fibrotic diseases, auto-immune diseases, and hepatitis C infection-mediated diseases, atherosclerosis, hypercholesterolemia and hyperlipidemia; cancer, glioblastoma, breast cancer, lymphoma, lung cancer; diabetes, metabolic disorders; myoblast differentiation; immune disorders. In particular embodiments, diseases, disorders, and conditions that can be treated using compositions and methods of the present invention include any disease process characterized by increased activity of one or more RNAs in one or more cell types, tissue types, and/or organs. Accordingly, any diseased cell, tissue, or organ wherein the disease is characterized by increased activity of one or more RNAs in is amenable to treatment.

Particular illustrative examples of diseases, disorders, and conditions suitable for treatment include cancer, atherosclerosis, hypercholesterolemia and hyperlipidemia, infectious disease, diabetes, metabolic disorders, immune and autoimmune disorders, inflammatory disease, organ disease, central nervous system (CNS) disease, and fibrotic disease.

In various embodiments, the terms “enhance,” “increase,” “stimulate,” facilitate,” “promote,” “heightens” when referring to oligomer activity, means the ability of an anti-miRNA molecule comprising at least one MGB to produce or cause a larger magnitude of physiological response (i.e., downstream effects) in a cell, as compared to the response caused an anti-miRNA molecule that lacks an MGB or a control molecule/composition. A measurable physiological response may include, for example, increased gene expression of two or more RNA targets sequences, e.g., genes, increased cell, tissue, or organ entry, increased physiological markers that signify improved organ function, increased cell-killing activity of a cytotoxic agent towards a cancer cell, increased tumor cell apoptosis, improvements in cancer-related symptoms, and others apparent from the understanding in the art and the description herein. An “increased” or “enhanced” amount is typically a “statistically significant” amount produced by an oligomer comprising an MGB, and can include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount produced by an oligomer that lacks an MGB moiety or a control composition.

As used herein, the term “miRNA target gene” refers to a gene that is regulated at the transcriptional, post-transcriptional, or translational level by a miRNA.

The terms “reduce,” “suppresses,” “decreases,” “inhibits,” “represses,” “lowers,” “abates,” or “lessens” relate generally to the ability of an oligomer comprising at least one MGB to cause a relevant, but smaller magnitude of physiological or cellular response in RNA expression and/or activity or a symptom of a relevant disease or condition that is associated with increased RNA expression or activity (e.g., cancer, hepatitis, immune disorders, metabolic disease, infectious disease, cardiovascular disease, fibrotic disease), as measured according to routine techniques in the diagnostic art. A “decreased” or “reduced” response can be a “statistically significant” decreased or reduced amount of response produced by an oligomer comprising an MGB compared to the response produced by an oligomer that is not conjugated or linked to an MGB, or a control composition, and can include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

As used herein, the terms “treat,” “treating,” and “treatment” refer to therapeutic or preventative measures described herein. The methods of “treatment” include administration of an oligomer (e.g., comprising at least one MGB) to a subject in order to inhibit, prevent, reverse (cure), delay, reduce the severity of, reduce the progression of or ameliorate one or more symptoms of a disease, disorder or condition characterized by an increase in RNA expression or activity. In one embodiment, the goal of treatment can be to improve the quality of life and prolong the survival of a subject beyond that expected in the absence of such treatment. In a particular embodiment, the efficacy of treatment ranges from amelioration of symptoms to complete reversal or cure of a disease, disorder, or condition associated with an increase in RNA, as discussed elsewhere herein. In a non-limiting example, the efficacy of treatment and progress thereof may be measured by performing organ function tests and monitoring physiological indicators of the particular disease, disorder, or condition being treated, as routinely practice in the art.

In particular illustrative embodiment, the present invention contemplates, in part, a method of treating a subject having a disease, disorder or condition associated with increased activity of one or more RNAs expressed in two or more cell types, tissue types, or organs comprises the steps of: identifying one or more RNAs expressed in two or more cell types, tissue types, or organs and having activity in diseased or affected cells, tissues, or organs (e.g., target cell, tissue, organ) and administering an oligomer that hybridizes to the target RNA. In preferred embodiment, the oligomer comprises one or more MGB moieties.

As used herein, the terms “affected cells, tissues, or organs” or “diseased cells, tissues, or organs” refer to cells, tissues, or organs that are the object of the treatment. The affected or diseased cells, tissues, or organs comprise activity of one or more RNAs. Affected or diseased cells, tissue, or organs may be targeted for treatment using the inventive oligomers and the methods described herein.

As used herein, the terms “normal cells, tissue, or organs” or “unaffected cells, tissues, or organs” refers to cells, tissues, or organs that do not have a disease, disorder, or condition associated with activity of one or more oligomers. In particular embodiments, normal cells, tissues, or organs are used for comparative purposes to determine a baseline from which to measure the extent to which a cell, tissue, or organ is diseased or affected by the RNA expression.

3. Measuring RNA Expression and Activity

In particular embodiments, the present invention provides a method of treating a subject affected by a disease, disorder, or condition associated with increased expression and/or activity of one or more RNAs in one or more cells, tissues, or organs. One having ordinary skill in the art would recognize that increased miRNA activity would be associated with a decreased expression of the known or suspected targets of the miRNA. Both the miRNA activity and gene/protein expression of the known or suspected targets of the miRNA can be measured using techniques known in the art.

For example, RNA activity and/or expression levels of the known or suspected targets of the RNA can be quantified by, e.g., Northern blot analysis, competitive polymerase chain reaction (PCR), or quantitative real-time PCR. RNA analysis can be performed on total cellular RNA or poly(A)⁺ mRNA. Methods of RNA isolation are well known in the art. Northern blot analysis is also routine in the art. Quantitative real-time PCR can be conveniently accomplished using the commercially available ABI PRISM® 7600, 7700, or 7900 Sequence Detection System, available from PE-Applied Biosystems, Foster City, Calif. and used according to manufacturer's instructions.

Other exemplary methods of measuring RNA activity and/or expression levels of the known or suspected targets of the RNA can be quantified include DNA arrays or microarrays (Brazma and Vilo, FEBS Lett., 2000, 480, 17-24; Celis, et al., FEBS Lett., 2000, 480, 2-16), SAGE (serial analysis of gene expression) (Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), and mass spectrometry methods (To, Comb. Chem. High Throughput Screen, 2000, 3, 235-41).

In particular embodiments, RNA expression can be assessed using in vitro luciferase reporter assays. In one non-limiting example, the activity of oligomers designed to inhibit particular RNAs can be evaluated in vitro using a DUAL-LUCIFERASE® Reporter Assay (Promega, Madison, Wis.) in which luciferase activity is inhibited by normal RNA expression (i.e., binding to its complementary sequence). In addition, an oligomer designed to inhibit one or more particular miRNAs one or more cell types, tissues, or organs will prevent the target miRNA from binding to its complementary sequence in the luciferase reporter, thus promoting luciferase activity. The luciferase reporter can be engineered using a miRNA sequence of interest.

Additionally, RNA activity and/or expression levels of the known can be quantified at the protein level. In addition, protein levels of a downstream target of a miRNA can be quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

4. Diseases, Disorders, and Conditions

Oligomers of the present invention have increased biodistribution compared to oligomers that are not conjugated to one or more MGBs or that lack MGBs. Certain embodiments of the present oligomer technology are not limited to any particular cell types. Particular embodiments of the present oligomer technology are useful for achieving broad tissue distribution among various cell types. Accordingly, oligomers of the invention can be tailored to any number of different cell types, tissues, or organs, e.g., 1, 2, 3, 4, 5, 6, 7, 8, 9, 10, or more or all cell types, tissues, and/or organs that require delivery of the oligomers.

Exemplary cells for use with the methods and compositions disclosed herein, include, but are not limited to, cancer cells, immune cells, epithelial cells, endothelial cells, mesodermal cells, and mesenchymal cells, bone cells, hematopoietic cells, skin cells, hair cells, eye cells, neural cells, glial cells, muscle cells (e.g., skeletal, cardiac, smooth muscle cells), meningeal cells, breast cells, liver cells, kidney cells, pancreatic cells, gastric cells, intestinal cells, colon cells, prostate cells, cervical cells, vaginal cells.

Exemplary tissues for use with the methods and compositions disclosed herein, include, but are not limited to, mesodermal tissue, connective tissue, smooth muscle tissue, striated muscle tissue, cardiac muscle tissue, bone tissue, bone marrow tissue, bone spongy tissue, cartilage tissue, adipose tissue, endodermal tissue, lung tissue, vascular tissue, pancreatic tissue, liver tissue, pancreatic ductal tissue, spleen tissue, thymus tissue, tonsil tissue, Peyer's patch tissue, lymph nodes tissue, thyroid tissue, endothelial tissue, blood cells, bladder tissue, kidney tissue, digestive tract tissue, esophagus tissue, stomach tissue, small intestine tissue, large intestine tissue, uterus tissue, testicular tissue, ovarian tissue, prostate tissue, endocrine tissue, mesentery tissue, and umbilical tissue, ectodermal tissue, epidermis tissue, dermis tissue, eye tissue, and nervous system tissue.

Exemplary organs for use with the methods and compositions disclosed herein, include, but are not limited to, bladder, bone, brain, breast, cartilage, cervix, colon, cornea, eye, nervous tissue, glia, esophagus, fallopian tube, heart, pancreas, intestines, gallbladder, kidney, liver, lung, ovaries, pancreas, parathyroid gland, pineal gland, pituitary gland, prostate, spinal cord, spleen, skeletal muscle, skin, smooth muscle, stomach, testes, thymus, thyroid, trachea, urogenital tract, ureter, urethra, uterus, and vagina.

Particular illustrative examples of diseases, disorders, and conditions suitable for treatment include tumor mediated angiogenesis, cancer, atherosclerosis, hypercholesterolemia and hyperlipidemia, infectious disease, diabetes, metabolic disorders, immune and autoimmune disorders, inflammatory disease, organ disease, central nervous system (CNS) disease, and fibrotic disease.

Exemplary cancers that may be treated using the methods and compositions herein, include, but are not limited to, lymphoreticular neoplasia, lymphoblastic leukemia, brain tumors, gastric tumors, plasmacytomas, multiple myeloma, leukemia, connective tissue tumors, lymphomas, and solid tumors.

Other suitable cancers that can be treated include, without limitation: carcinomas, e.g., malignant melanoma, basal cell carcinoma, ovarian carcinoma, breast carcinoma, non-small cell lung cancer, renal cell carcinoma, bladder carcinoma, recurrent superficial bladder cancer, hepatic carcinoma, stomach carcinoma, prostatic carcinoma, pancreatic carcinoma, lung carcinoma, cervical carcinoma, cervical dysplasia, laryngeal papillomatosis, colon carcinoma, colorectal carcinoma carcinoid tumors; sarcomas, e.g., osteosarcoma, Ewing's sarcoma, chondrosarcoma, malignant fibrous histiocytoma, fibrosarcoma and Kaposi's sarcoma; and glioblastomas.

In certain embodiments, when treating cancer, one or more adjunctive therapies or medicaments may be administered in combination with an oligomers according to the present invention. The adjunctive therapy may be co-administered with the oligomers (in the same or in different compositions and at the same or at different administration sites). The adjunctive therapy can also be administered before or after the administration of the oligomers.

Exemplary adjunctive therapies include, without limitation, adrenocorticosteroids, such as prednisone, dexamethasone or decadron; altretamine (hexalen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifin (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine). Suitably, the further chemotherapeutic agent is selected from taxanes such as Taxol, Paclitaxel or Docetaxel.

Exemplary infectious diseases that may be treated using the methods and compositions herein, include, but are not limited to, HIV infection, CMV infection, HSV infection, diphtheria, tetanus, pertussis, polio, hepatitis B, hepatitis C, hemophilus influenza, measles, mumps, and rubella.

Exemplary inflammatory diseases that may be treated using the methods and compositions herein, include, but are not limited to, rheumatoid arthritis, systemic lupus erythematous (SLE) or Lupus, multiple sclerosis (MS), myasthenia gravis (MG), scleroderma, polymyositis, inflammatory bowel disease, dermatomyositis, ulcerative colitis, Crohn's disease, vasculitis, psoriatic arthritis, exfoliative psoriatic dermatitis, pemphigus vulgaris and Sjorgren's syndrome, in particular inflammatory bowel disease Crohn's disease, bursitis, synovitis, capsulitis, tendinitis and/or other inflammatory lesions of traumatic and/or sportive origin.

Exemplary metabolic disease (a disorder caused by the accumulation of chemicals produced naturally in the body) that may be treated using the methods and compositions herein, include, but are not limited to, Crigler Najjar Syndrome, diabetes, fatty acid oxidation disorders, galactosemia, glucose-6-phosphate dehydrogenase (G6PD) deficiency, glutaric aciduria, glutaric acidemia Type I, glutaric acidemia, Type II, F-HYPDRR-familial hypophosphatemia, vitamin D resistant Rickets, Krabbe disease, long chain 3 hydroxyacyl CoA dehydrogenase deficiency (LCHAD), mannosidosis, maple syrup urine disease, mitochondrial disorders, mucopolysaccharidosis syndromes: Niemann Pick, organic acidemias, PKU, Pompe disease, porphyria, metabolic syndrome, hyperlipidemia and inherited lipid disorders, trimethylaminuria: the fish malodor syndrome, and urea cycle disorders.

Exemplary diseases, disorders, and conditions of the nervous system that may be treated using the methods and compositions herein, include, but are not limited to: spinal cord injury; head trauma or surgery; viral infections; neurodegenerative diseases, e.g., AIDS dementia complex; demyelinating diseases, e.g., multiple sclerosis and acute transferase myelitis; extrapyramidal and cerebellar disorders, e.g., lesions of the ecorticospinal system; disorders of the basal ganglia or cerebellar disorders; hyperkinetic movement disorders, e.g., Huntington's chorea and senile chorea; drug-induced movement disorders, e.g., induced by drugs that block CNS dopamine receptors; hypokinetic movement disorders, e.g., Parkinson's disease; progressive supra-nucleo palsy; structural lesions of the cerebellum; spinocerebellar degenerations, e.g., spinal ataxia, Friedreich's ataxia, cerebellar cortical degenerations, multiple systems degenerations (Mencel, Dejerine Thomas, Shi-Drager, and Machado-Joseph), systemic disorders, e.g., Rufsum's disease, abetalipoproteinemia, ataxia, telangiectasia; and mitochondrial multi-system disorder; demyelinating core disorders, e.g., multiple sclerosis, acute transverse myelitis; and disorders of the motor unit, e.g., neurogenic muscular atrophies (anterior horn cell degeneration, e.g., amyotrophic lateral sclerosis, infantile spinal muscular atrophy and juvenile spinal muscular atrophy); Alzheimer's disease; Down's Syndrome in middle age; Diffuse Lewy body disease; senile demetia of Lewy body type; Wernicke-Korsakoff syndrome; chronic alcoholism; Creutzfeldt-Jakob disease; subacute sclerosing panencephalitis Hallerrorden-Spatz disease; and dementia pugilistica.

Exemplary fibroproliferative diseases include, that may be treated using the methods and compositions herein, include, but are not limited to, scleroderma (including morphea, generalized morphea, or linear scleroderma), kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), liver fibrosis (including cirrhosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Hansen's disease, collagenous colitis, acute fibrosis, systemic sclerosis, and fibrosis arising from tissue or organ transplant or graft rejection.

Illustrative examples of miRNA targets and corresponding diseases, disorders, and conditions suitable for treatment by the compositions of the invention include, but are not limited to: miR-1, cardiac arrhythmia; miR-21, glioblastoma, breast cancer, hepatocellular carcinoma, colorectal cancer, sensitization of gliomas to cytotoxic drugs, and cardiac hypertrophy; miR-21, miR-200b, response to chemotherapy and regulation of miR-141, and cholangiocarcinoma growth; miR-122, hypercholesterolemia, hepatitis C infection, and hemochromatosis; miR-19b, lymphoma and other tumour types; miR-26a, osteoblast differentiation of human stem cells; miR-155, lymphoma, pancreatic tumor development, and breast and lung cancer; miR-203, psoriasis; miR-375, diabetes, metabolic disorders, and glucose-induced insulin secretion from pancreatic endocrine cells; miR-181, myoblast differentiation, and auto immune disorders, miR-10b, breast cancer cell invasion and metastasis; miR-125b-1, breast, lung, ovarian and cervical cancer; miR-221 and miR-222, prostate carcinoma, human thyroid papillary carcinoma, and human hepatocellular carcinoma; miRNA-372 and -373, testicular germ cell tumors; miR-142, B-cell leukemia; miR-17-19b, B-cell lymphomas, lung cancer, and hepatocellular carcinoma.

All publications, patents and patent applications cited herein, whether supra or infra, are hereby incorporated by reference in their entirety.

As used herein, the term “about” or “approximately” refers to a time period, quantity, level, value, number, frequency, percentage, dimension, size, amount, weight or length that varies by as much as 30, 25, 20, 25, 10, 9, 8, 7, 6, 5, 4, 3, 2 or 1% to a reference time period, quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

As used in this specification and the appended claims, the singular forms “a,” “an” and “the” include plural references unless the content clearly dictates otherwise.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Quantitative Whole-Body Autoradiography (QWBA) Analysis of Anti-miRNAs

Quantitative whole-body autoradiography (QWBA) analysis was performed to compare the tissue distribution and concentration parameters of conjugated anti-miRNAs, e.g., MI-01453 (SEQ ID NO: 2) and MI-01454 (SEQ ID NO: 3) to unconjugated anti-miRNAs, e.g., MI-01452 (SEQ ID NO: 1), following single dose IV administration of ¹⁴C labeled anti-miRNAs to male CD-1 mice. MI-01452, MI-01453, and MI-01454 are anti-miRNAs directed against miR-21 (see Table 2.

TABLE 2
anti-miRNAs

| Anti-miRNA | Anti-miRNA sequence |
|---|---|
| MI-01452 | 5′-L1-mU*mC*mAmAmCmAmUmCmAmGmUmCmUmG |
| | mAmUmAmAmG*mC*mU*mA-3′ |
| MI-01453 | 5′-CDPI₃-L1-mU*mC*mAmAmCmAmUmCmAmGmUmC |
| | mUmGmAmUmAmAmG*mC*mU*mA-3′ |
| MI-01454 | 5′-CDPI₄-L1-mU*mC*mAmAmCmAmUmCmAmGmUmC |
| | mUmGmAmUmAmAmG*mC*mU*mA-3′ |

To clarify the sequence composition of these anti-miRNAs, the lowercase “m” designates the incorporation of 2′-OMe on the sugar. The “*” designates the incorporation of a phosphorothioate linkage (PS) in place of a phosphodiester linkage. L1 is the linker and has the following composition: “—P(═O)(OH)O(CH2)₆NH—”.

### Methods

1. Animal Experimentation

All in-life portions of the study were conducted at Xenometrics, LLC (Study # XPK10-323); the methods in the protocol are briefly described below. Thirty adult male CD-1 mice (three groups of ten mice, with seven mice selected for QWBA per group) were used for the QWBA analysis. Each groups received a single IV administration of anti-miRNA in phosphate buffered saline (PBS) via tail vein injection. The dose administration information is summarized in the following table:

| | | | | | Dose: |
|---|---|---|---|---|---|
| Group | Anti-miRNA | Conjugate | Route | (μCi/kg) | (mg/kg) |
| 1 | [¹⁴C] MI-01452 | None | IV | 325 | 20 |
| 2 | [¹⁴C] MI-01453 | CDPI₃ | IV | 325 | 20 |
| 3 | [¹⁴C] MI-01454 | CDPI₄ | IV | 260 | 20 |

Two mice per group per time point were euthanized at 0.167, 0.5, 1, 2, 4, 8, and 24 hours (h) and 3, 7, and 14 days post-dose. Animals were deeply anesthetized and then euthanized by freezing in a hexane/dry-ice bath.

2. Whole-Body Autoradiography

Each frozen mouse carcass was embedded in a 2% carboxymethylcellulose matrix and mounted on a microtome stage (Leica CM3600 Cryomacrocut, Nussloch, Germany; or Vibratome 9800, St. Louis, Mo.) maintained at approximately −20° C. Three quality control standards (QC), which were plasma fortified with [¹⁴C] glucose at one concentration (approximately 0.05 μCi/g), were placed into the frozen blocks prior to sectioning, and were used for section thickness quality control as per QPS SOP. Sections approximately 40 μm thick were taken in the sagittal plane, and captured on adhesive tape (Scotch Tape No. 8210, 3M Ltd., St. Paul, Minn., USA). Sections were collected to encompass the following tissues, organs and biological fluids: adipose (brown and white), adrenal gland, bile (in duct), blood (cardiac), brain (cerebrum, cerebellum, medulla), bone, bone marrow, cecum (and contents), epididymis, eye (uveal tract and lens), Harderian gland, heart, kidney (renal cortex and medulla), large intestine (and contents), liver, lung, lymph node, pancreas, pituitary gland, prostate gland, salivary gland, seminal vesicles, skeletal muscle, skin, stomach (gastric mucosa and contents), small intestine (and contents), spleen, spinal cord, testis, thymus, thyroid, and urinary bladder (and contents).

Sections were dried by sublimation in the cryomicrotome at −20° C. for at least 48 h. A set of sections were mounted on a cardboard backing, covered with a thin plastic wrap, and exposed along with calibration standards (14C-glucose mixed with blood at 10 different concentrations at approximately 0.0007 to 7.410 μCi/g) to a ¹⁴C-sensitive phosphor imaging plate (Fuji Biomedical, Stamford, Conn.). The imaging plate and sections were placed in light-tight exposure cassettes, in a copper-lined lead safe, at room temperature, for a 4-day exposure period. The imaging plates were scanned using the Typhoon 9410™ image acquisition system (GE Healthcare/Molecular Dynamics, Sunnyvale, Calif., USA) and the resultant images stored on a dedicated QPS computer server. Quantification was performed by image densitometry using MCID™ image analysis software (v. 6.0 or 7.0, Interfocus Imaging, Inc., Linton, Cambridge, UK), and a standard curve constructed from the integrated response (MDC/mm²) and the nominal concentrations of the ¹⁴C-calibration standards.

The concentrations of radioactivity were expressed as μCi/g and converted to μg equivalents of MI-01452, MI-01453, and MI-01454 per gram sample (μg equiv/g) using the specific activity of each administered test article (0.01976 μCi/μg for [¹⁴C] MI-01452, 0.01911 μCi/μg for [¹⁴C]MI-01453, or 0.01552 μCi/μg for [¹⁴C] MI-01454). A lower limit of quantification (LLOQ) was applied to the data. Concentrations of [¹⁴C]-radioactivity were expressed as the μg equivalents of MI-01452, MI-01453, or MI-01454 per gram sample (μg equiv/g). Tissue concentration values that fell below the lowest standard on the calibration curve were noted as below the quantifiable limit (BQL); and tissues that could not be visualized on the autoradiographic images during QWBA analysis were noted as not sampled (NS) and were considered to be BQL.

3. Standard Calculations

Response curves were generated using a weighted, 1^{st} degree, polynomial, linear equation (1/MDC/mm²). A numerical estimate of goodness of fit was given by the relative error, where the absolute value for the relative error of each calibration standard was ≦0.250 to be accepted.

### Standard Curve Calculations:

Response(MDC/mm²)=a₁×Concentration(Density-Standards in μCi/g)+a₀

### Where:

Density-Standards=concentration in μCi/g
MDC/mm²=Molecular Dynamic Counts/area of tissue
a₁=slope
a₀=y-intercept
   The relative error for each standard was calculated using the standard curve according to;

$$Relative Error = \frac{\begin{matrix}nominal concentration ( μCi \text{/} g ) \\ minus calculated concentration ( μCi \text{/} g )\end{matrix}}{nominal concentration ( μCi \text{/} g )}$$

Individual sample concentrations were then calculated according to:

$$Concentration ( μg equilvalents / g of tissue ) = \frac{Concentration from std . curve ( μCi \text{/} g )}{Specific Activity ( μCi \text{/} ng )}$$

### MI-01452

The LLOQ and upper limit of quantitation (ULOQ) were based on the lowest (0.000706 μCi/g) and highest (7.41040495 μCi/g) standards used in the calibration curve. For this study the LLOQ was 0.036 μg equiv/g of tissue and the ULOQ was 375.02 μg equiv/g of tissue:

$$LLOQ = \frac{Density - Standard ( 0.000706 μCi \text{/} g )}{Specific Activity ( 0.01976 μCi \text{/} μg )}$$

$$ULOQ = \frac{Density - Standard ( 7.41040495 μCi \text{/} g )}{Specific Activity ( 0.01976 μCi \text{/} μg )}$$

### MI-01453

The LLOQ and upper limit of quantitation (ULOQ) were based on the lowest (0.000706 μCi/g) and highest (7.41040495 μCi/g) standards used in the calibration curve. For this study the LLOQ was 0.037 μg equiv/g of tissue and the ULOQ was 387.776 μg equiv/g of tissue:

$$LLOQ = \frac{Density - Standard ( 0.000706 μCi \text{/} g )}{Specific Activity ( 0.01911 μCi \text{/} μg )}$$

$$ULOQ = \frac{Density - Standard ( 7.41040495 μCi \text{/} g )}{Specific Activity ( 0.01911 μCi \text{/} μg )}$$

### MI-01454

The LLOQ and upper limit of quantitation (ULOQ) were based on the lowest (0.000706 μCi/g) and highest (7.41040495 μCi/g) standards used in the calibration curve. For this study the LLOQ was 0.045 μg equiv/g of tissue and the ULOQ was 477.475 μg equiv/g of tissue:

$$LLOQ = \frac{Density - Standard ( 0.000706 μCi \text{/} g )}{Specific Activity ( 0.01552 μCi \text{/} μg )}$$

$$ULOQ = \frac{Density - Standard ( 7.41040495 μCi \text{/} g )}{Specific Activity ( 0.01552 μCi \text{/} μg )} .$$

### Results and Discussion

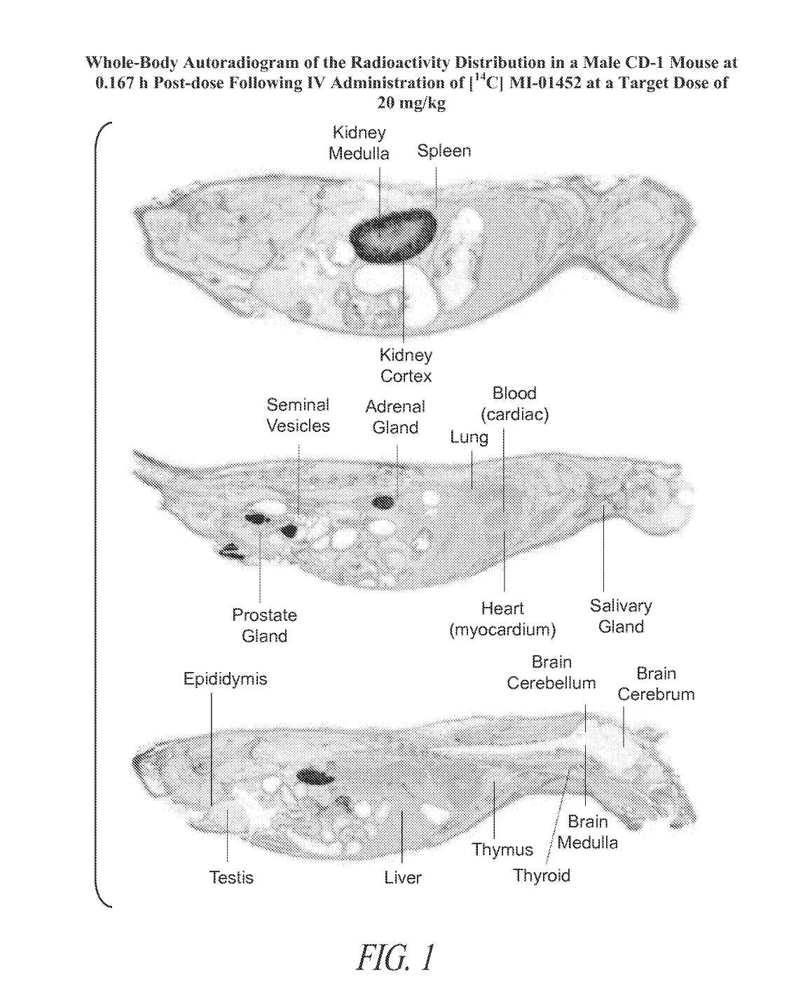

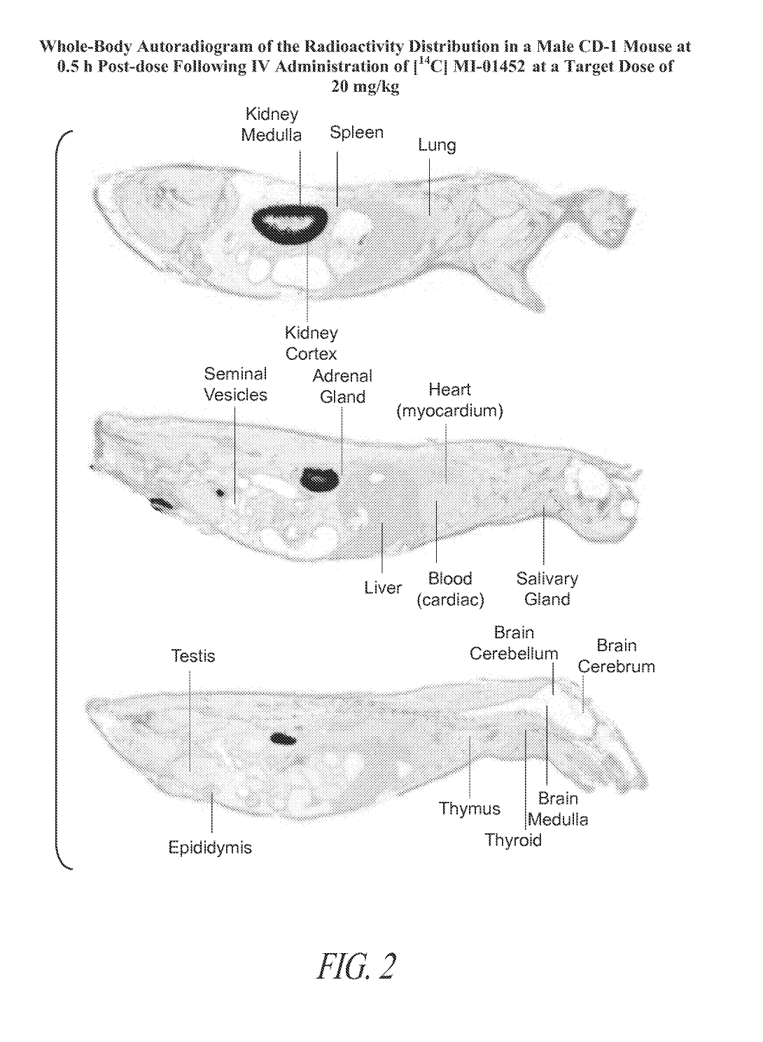

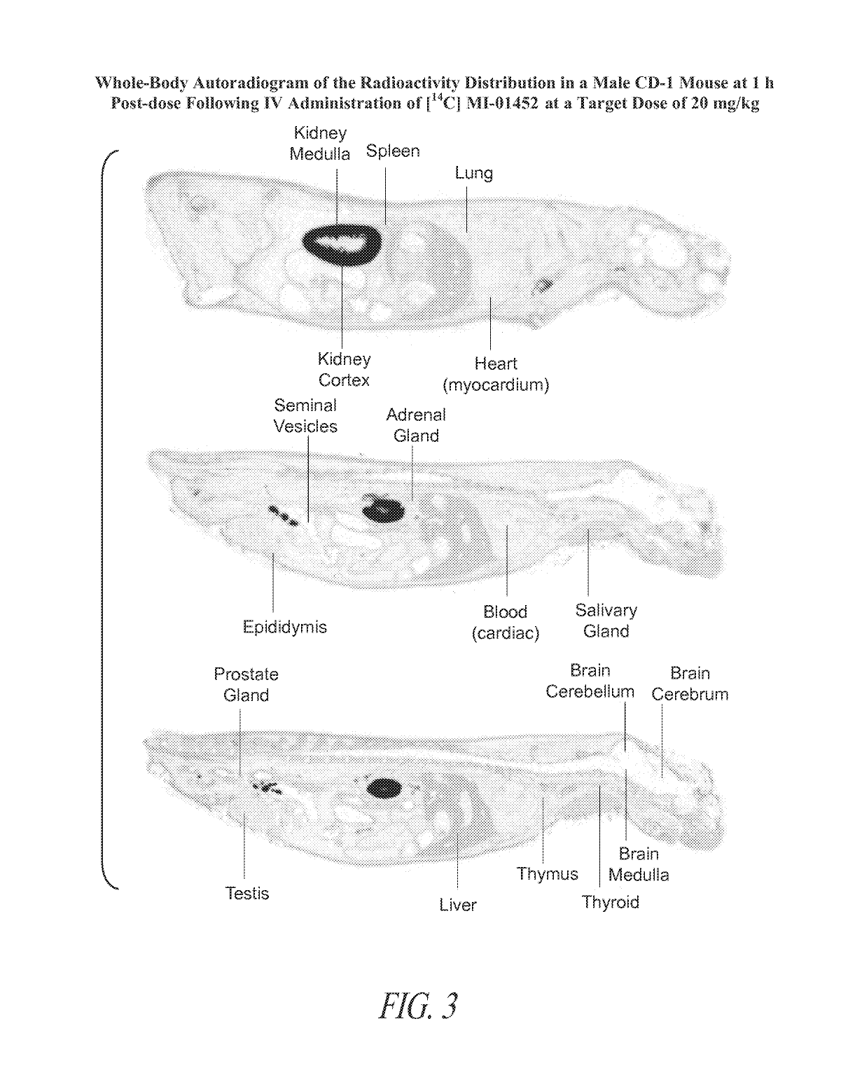

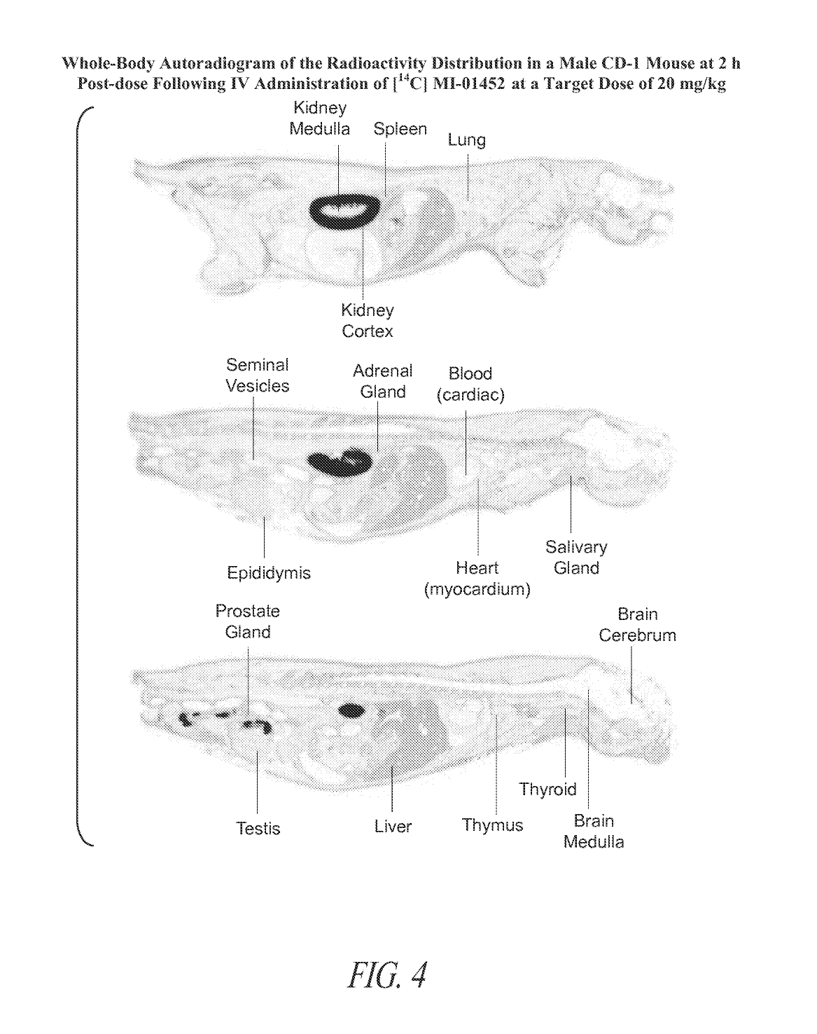

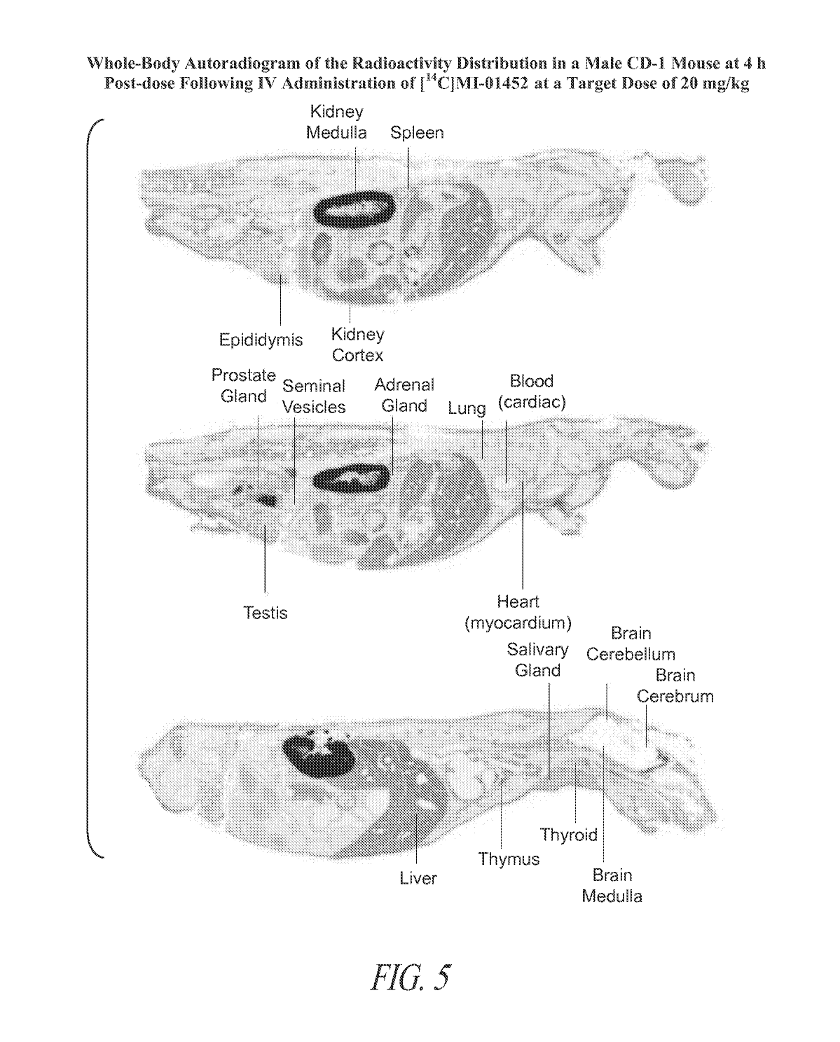

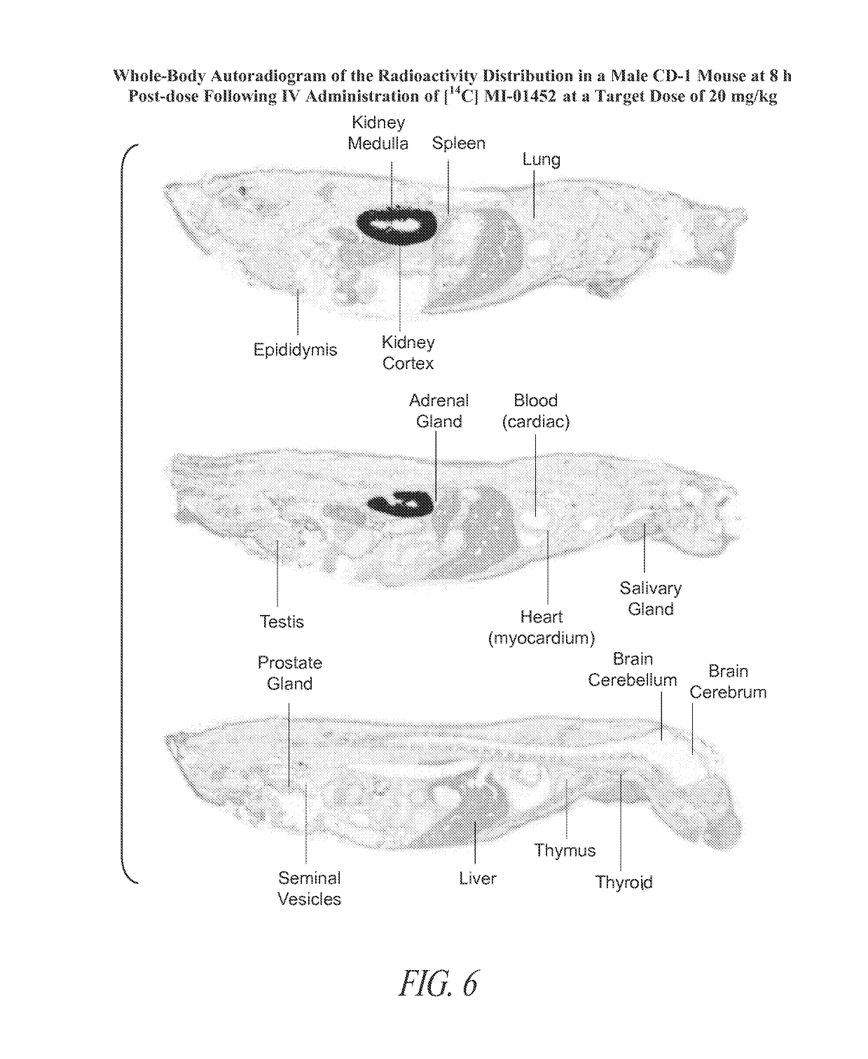

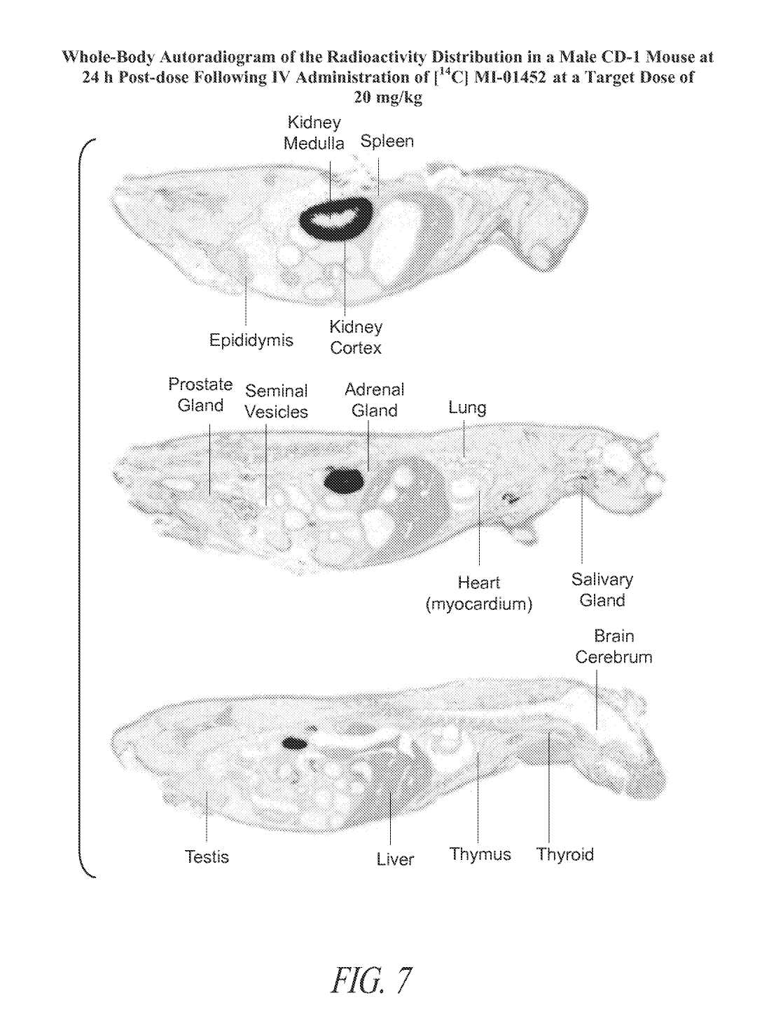

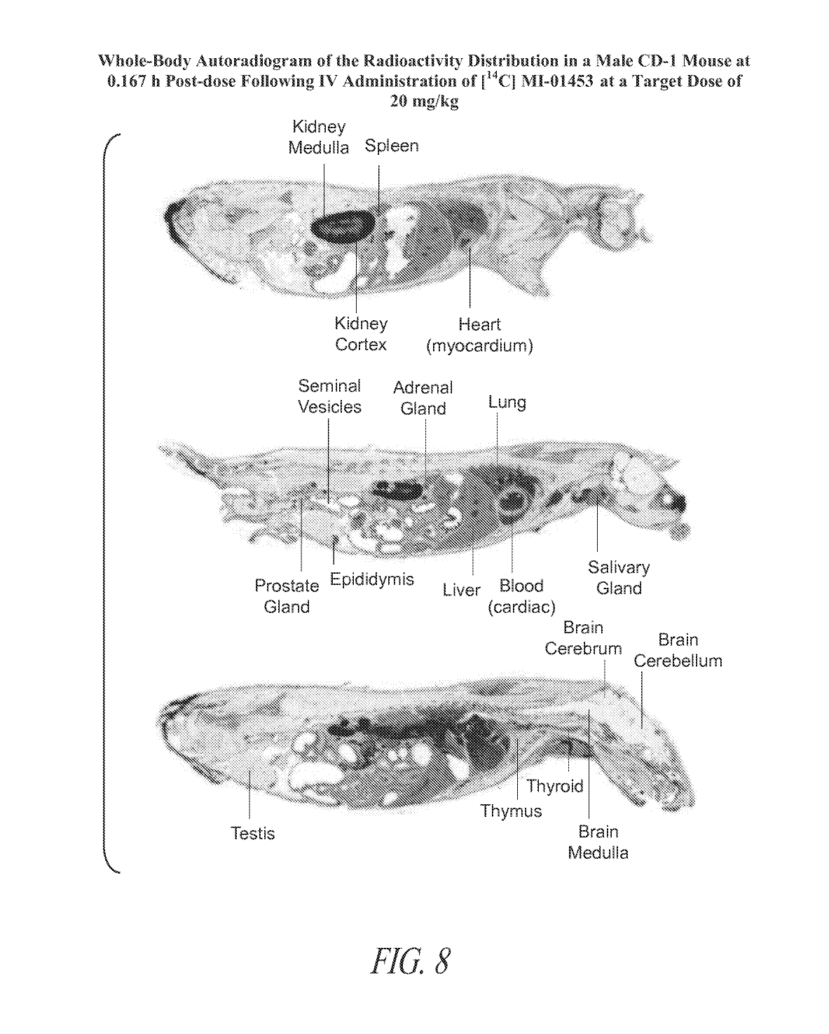

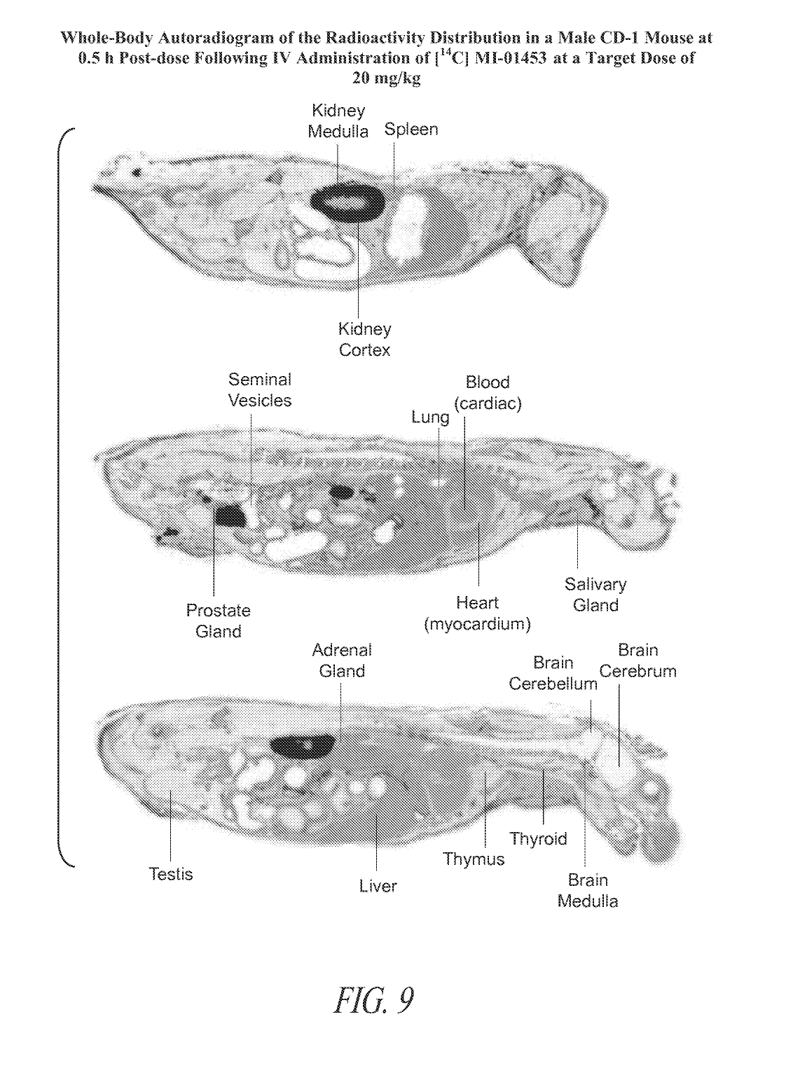

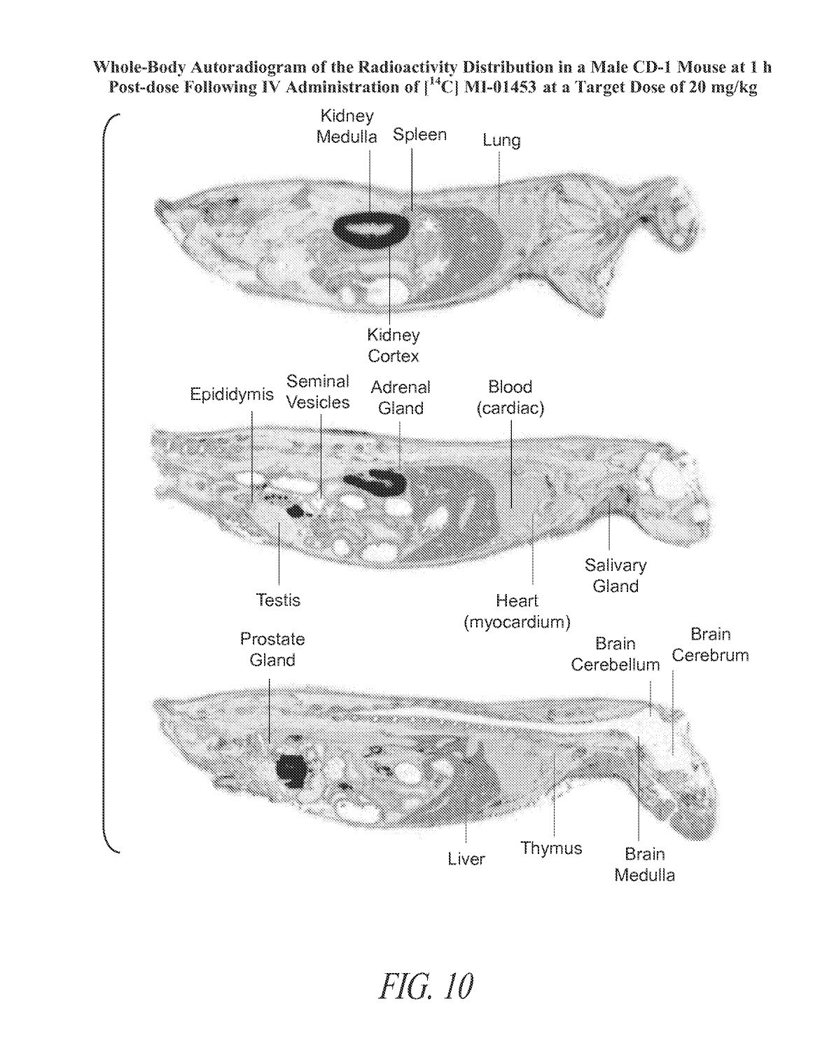

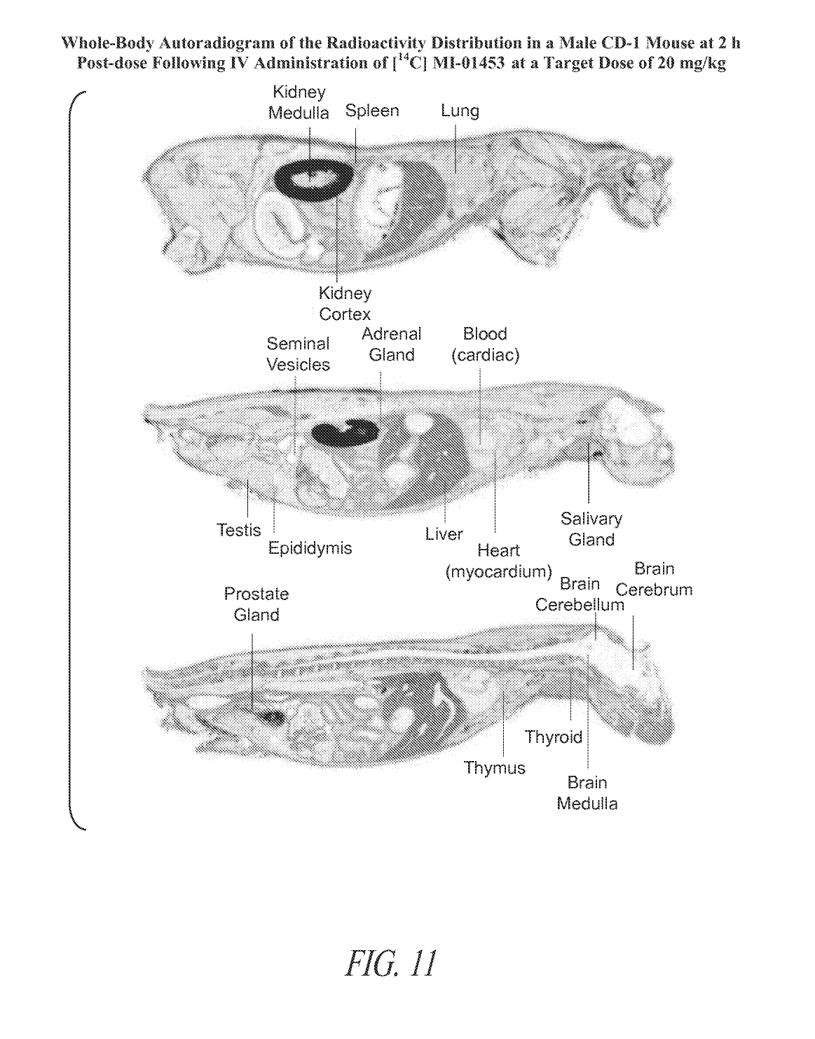

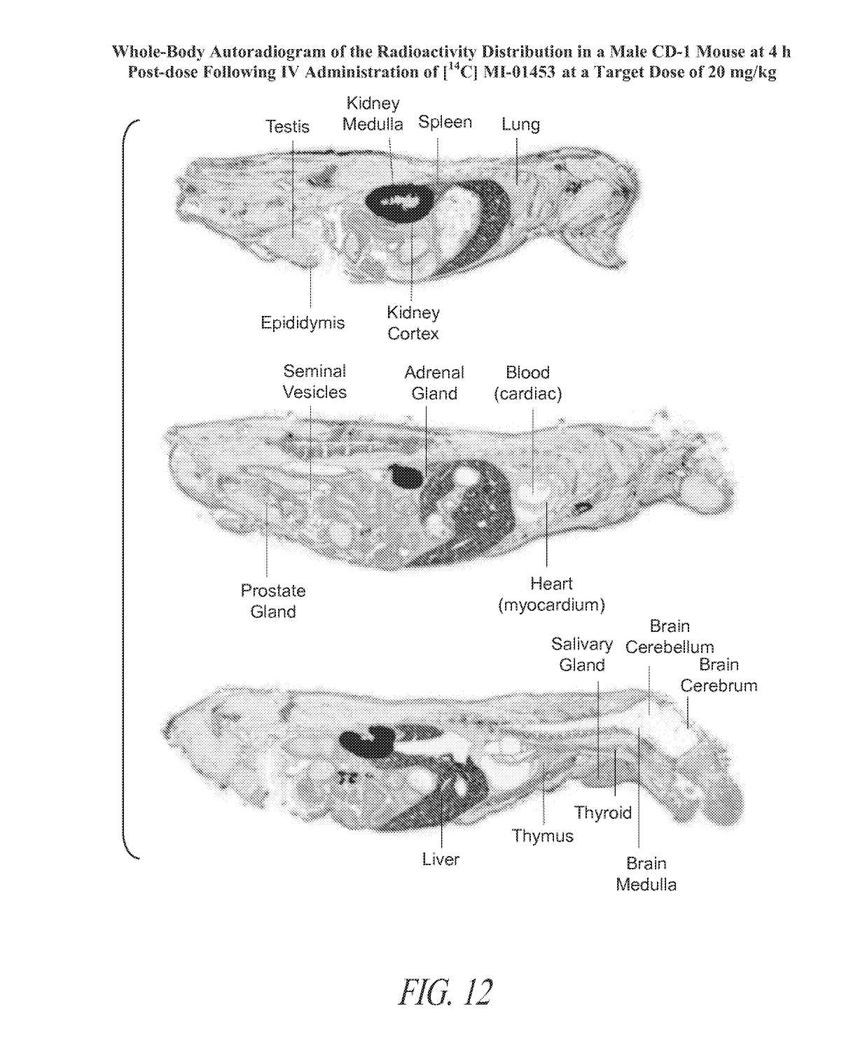

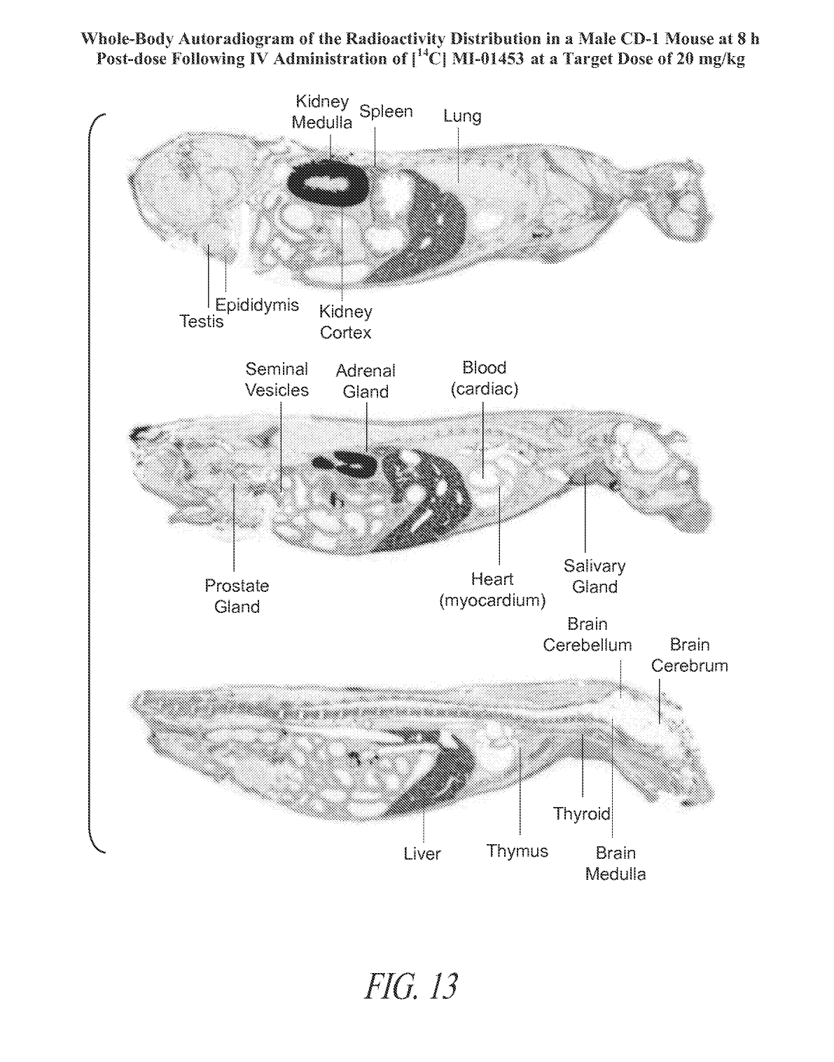

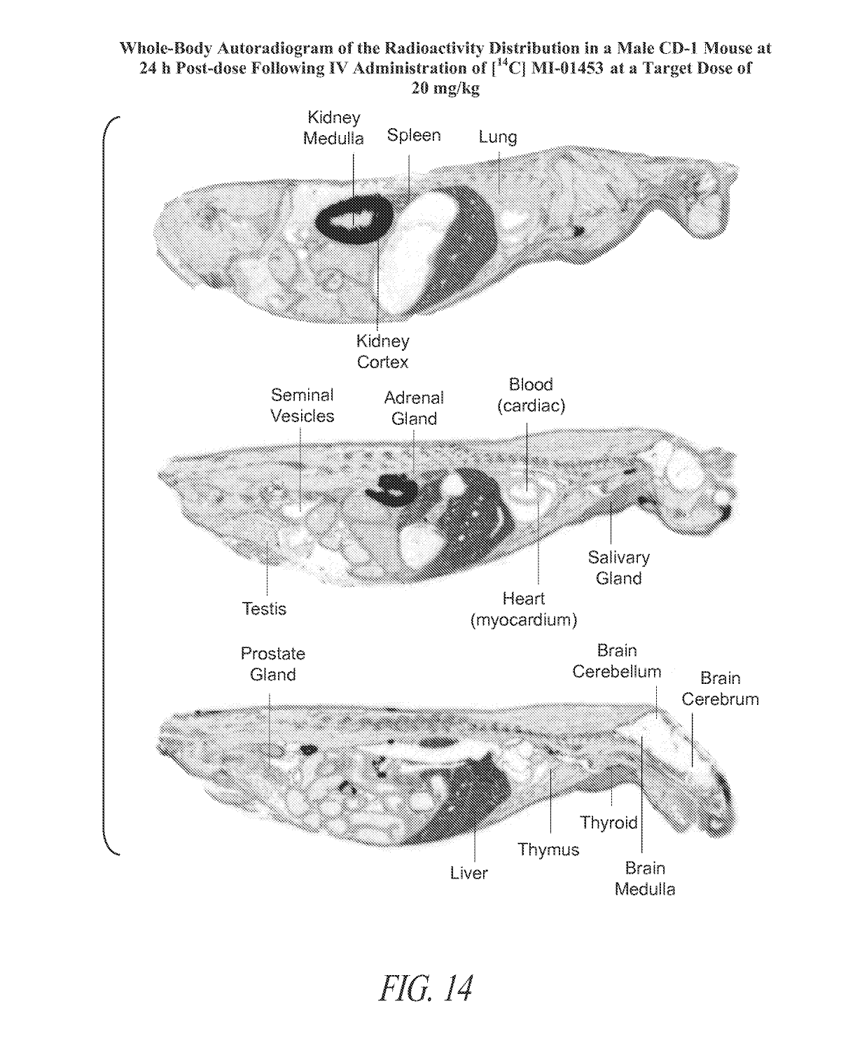

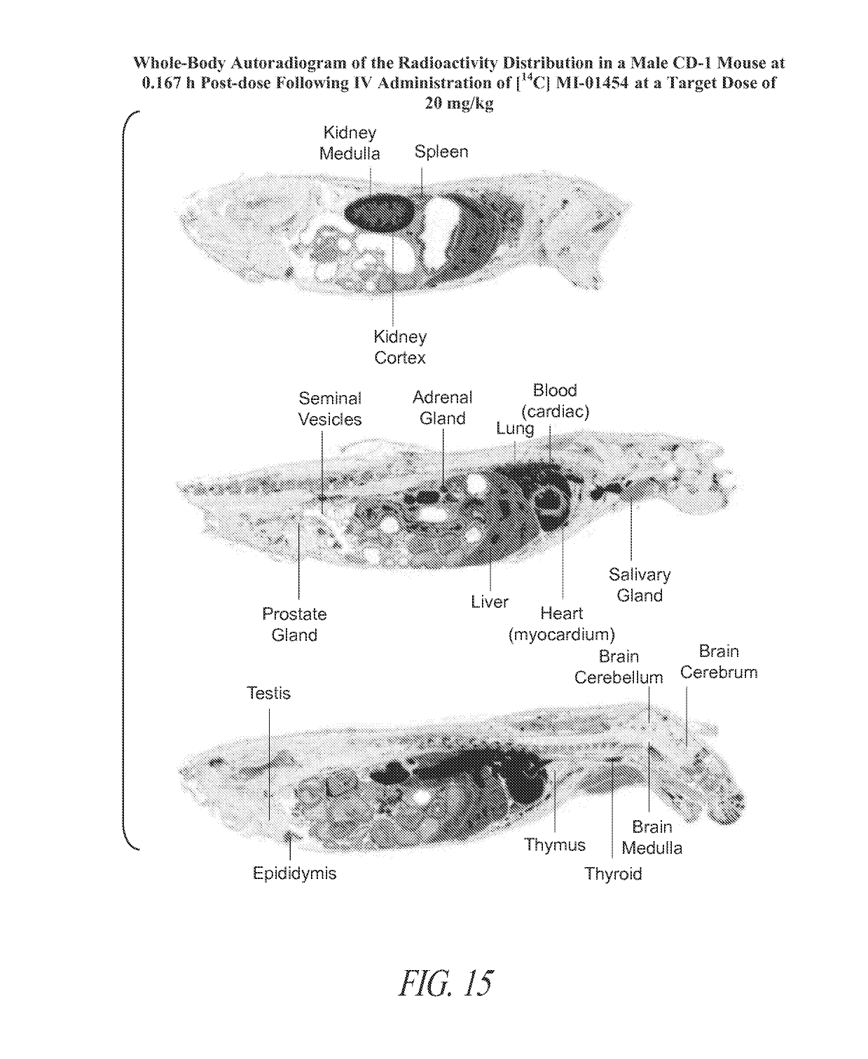

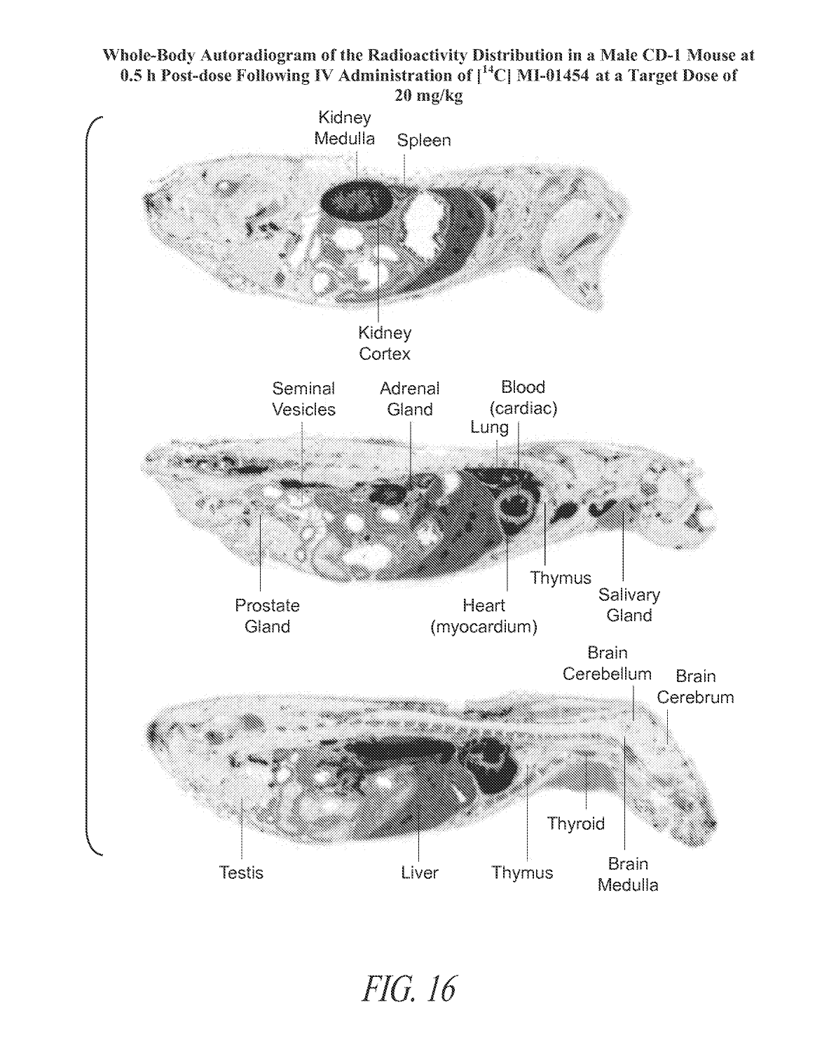

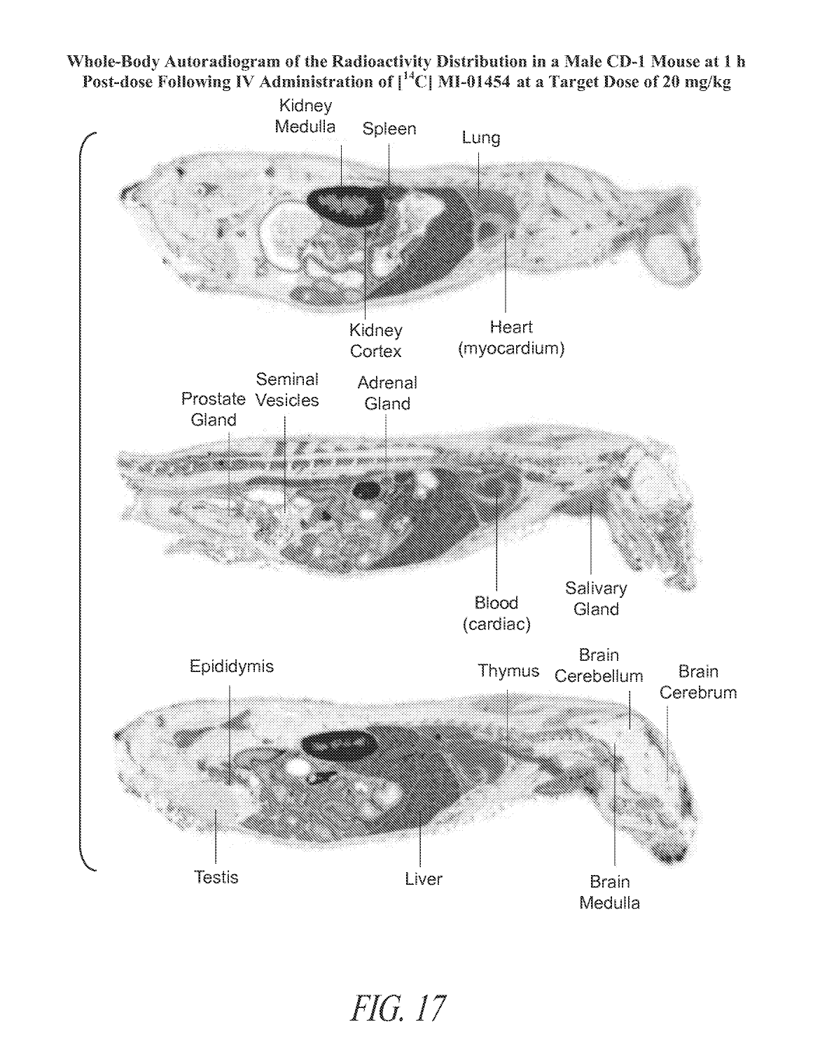

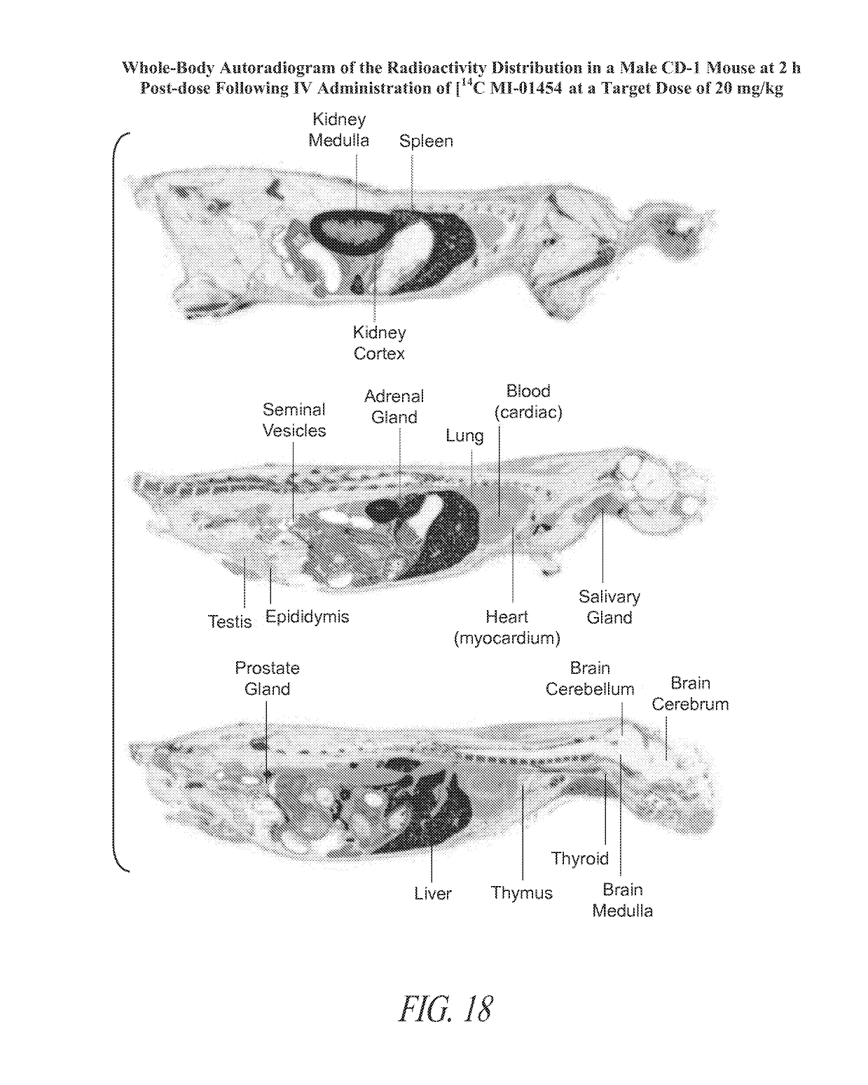

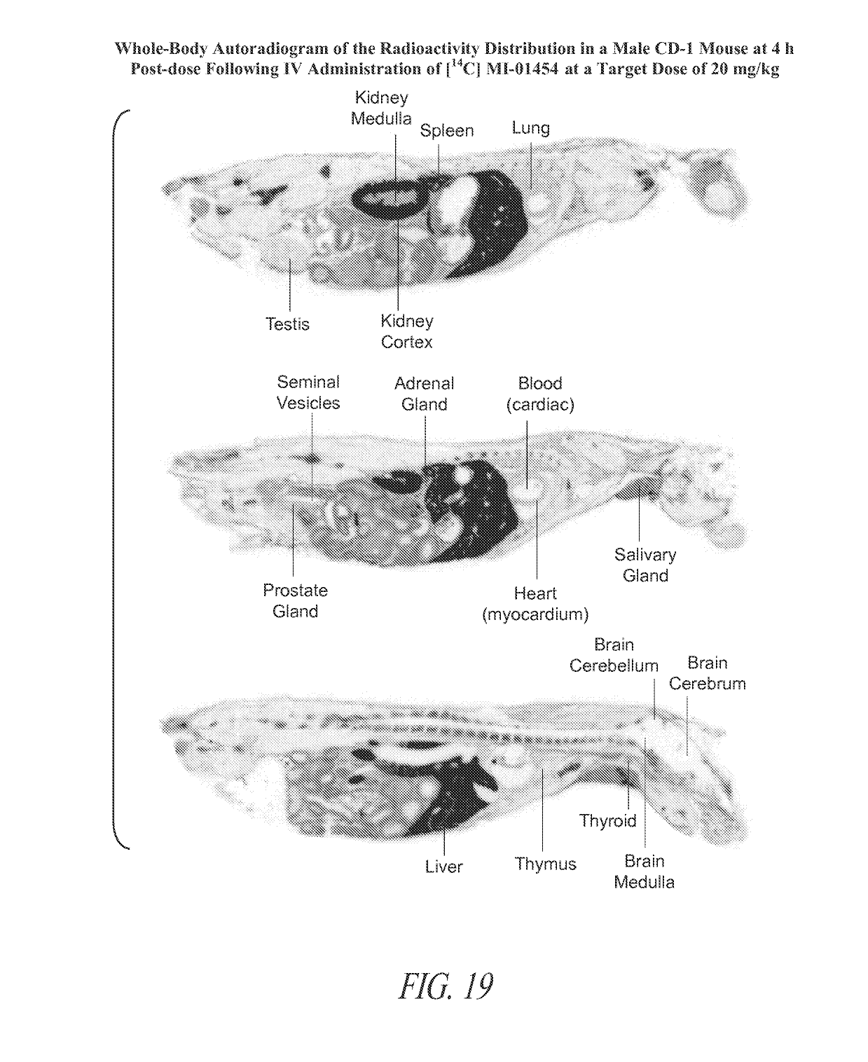

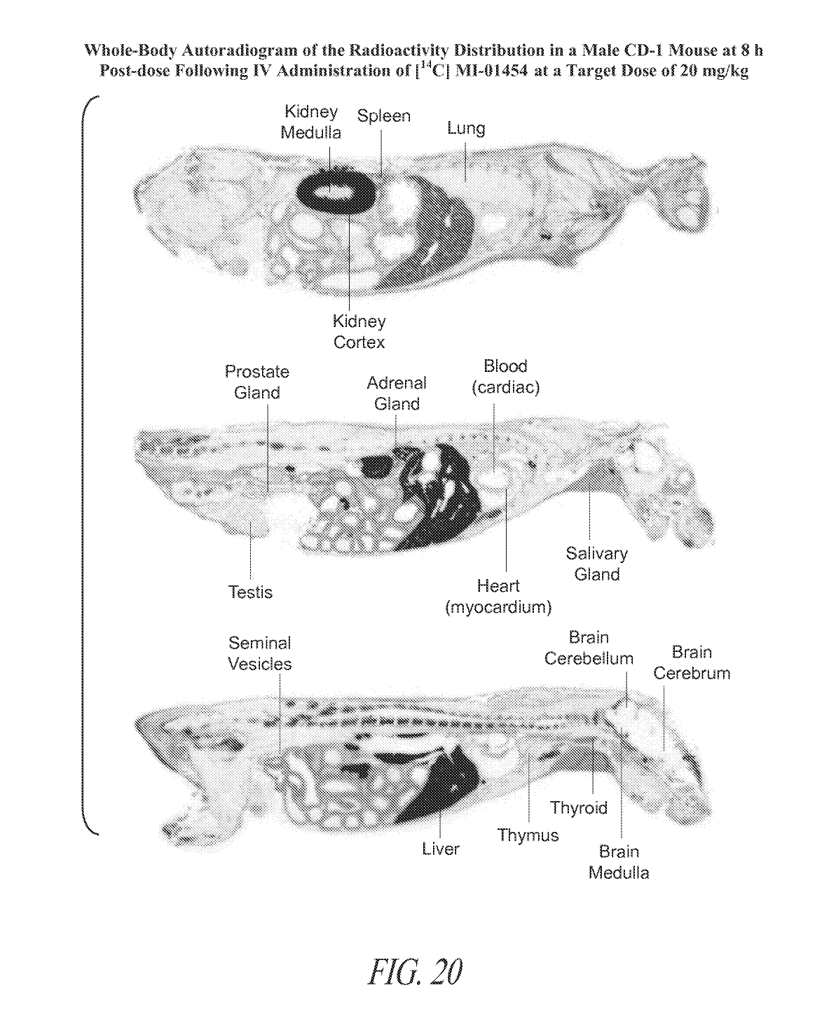

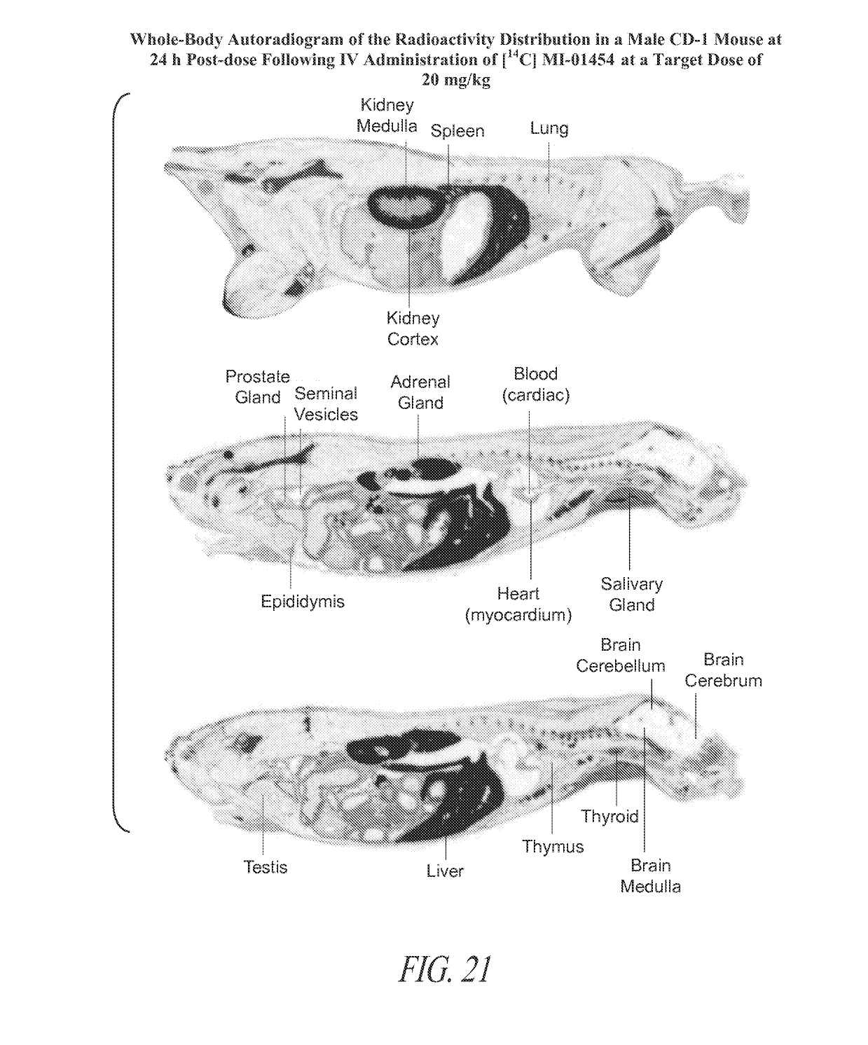

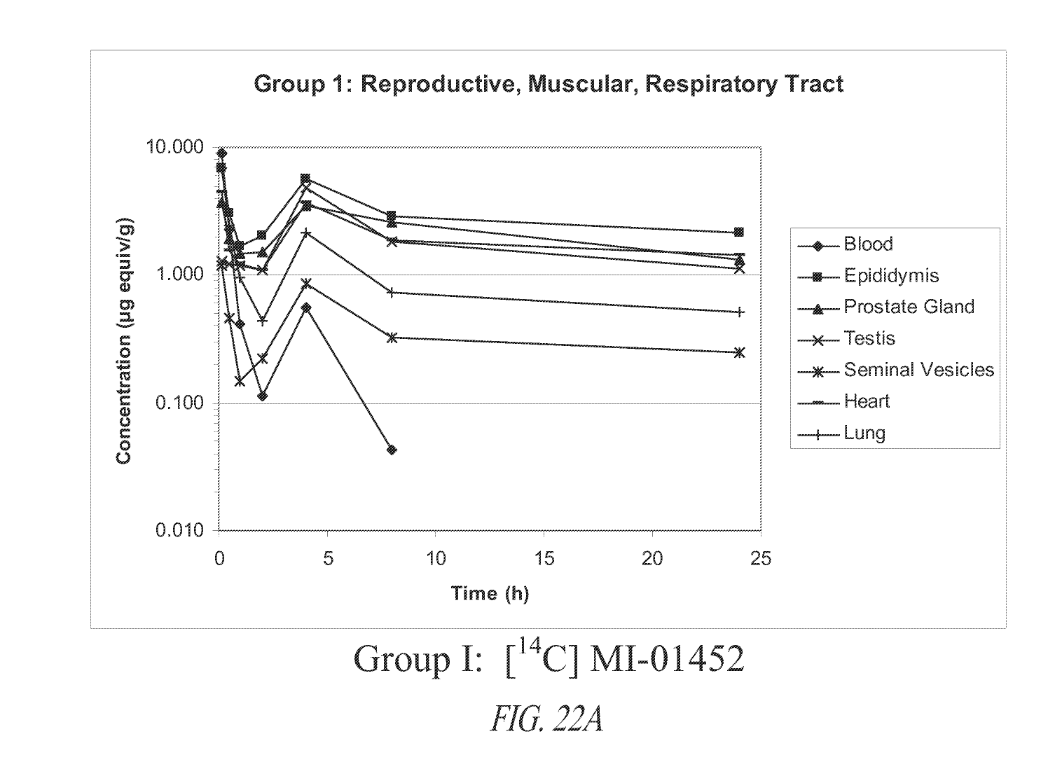

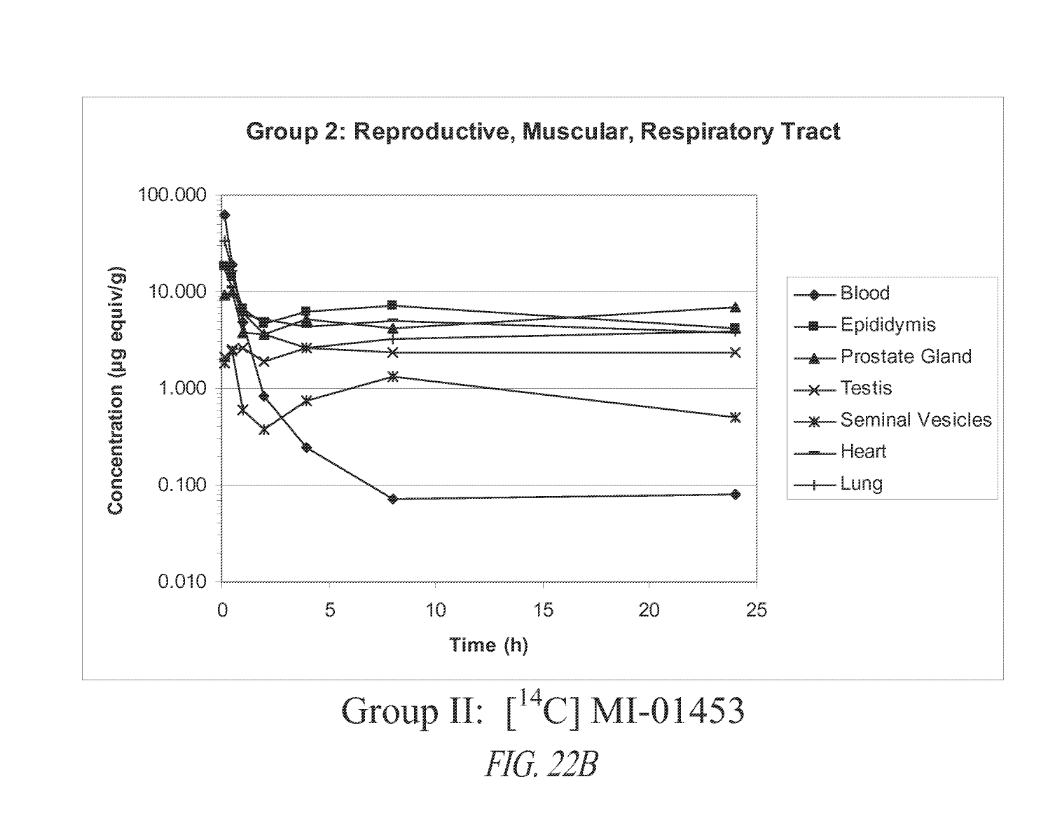

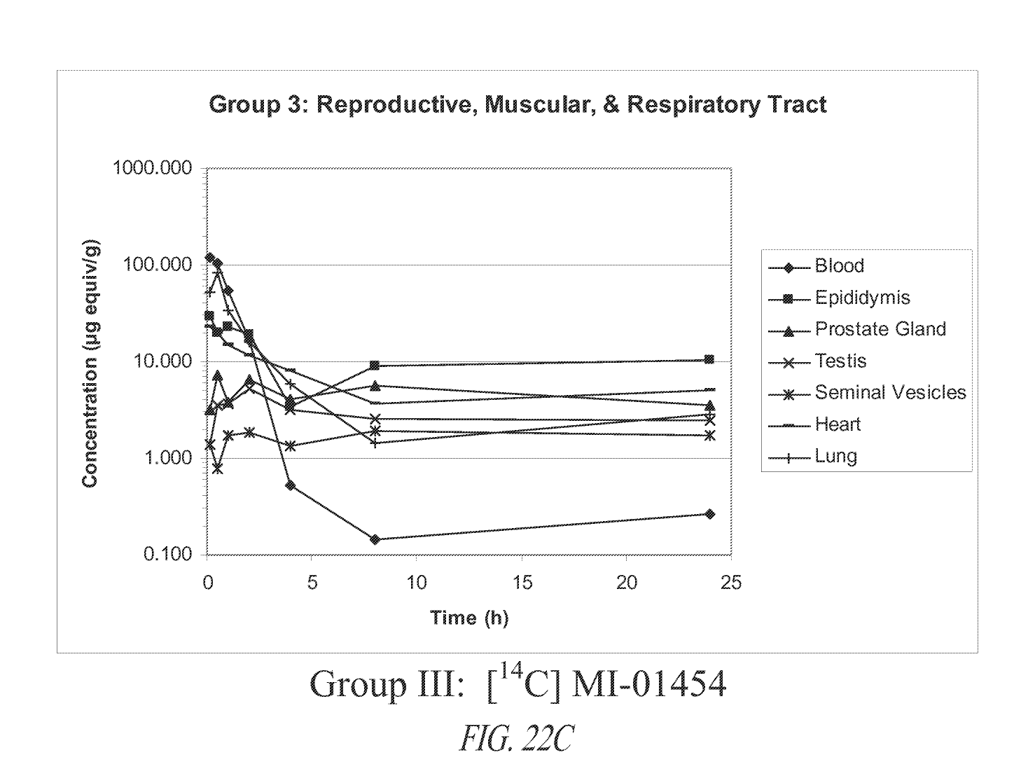

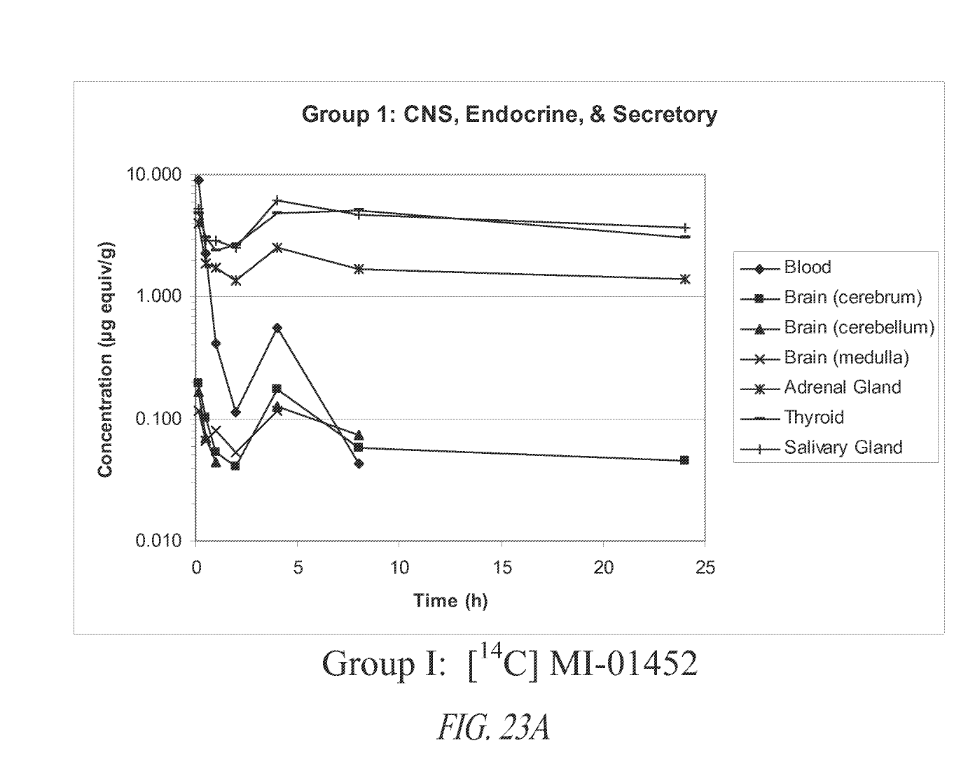

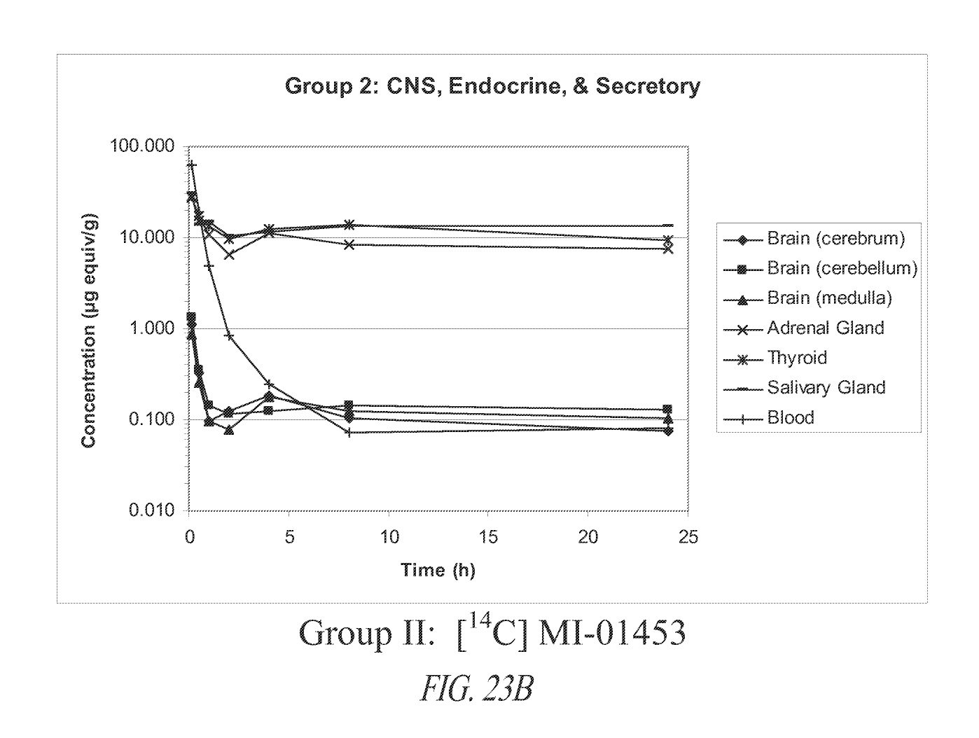

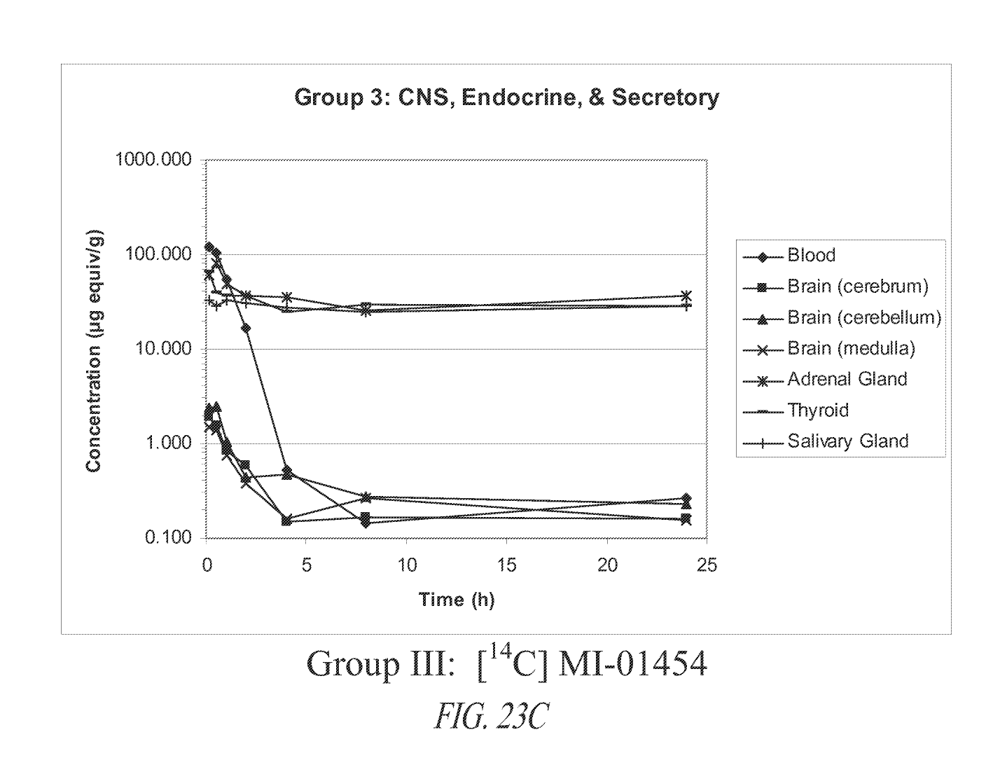

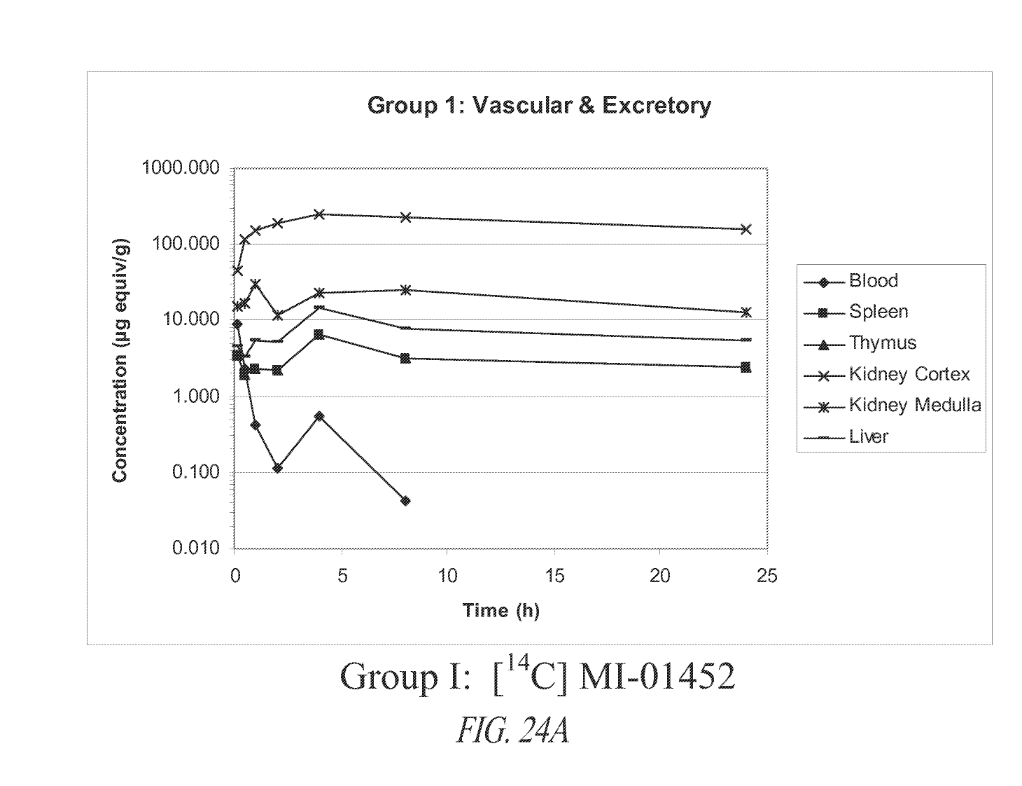

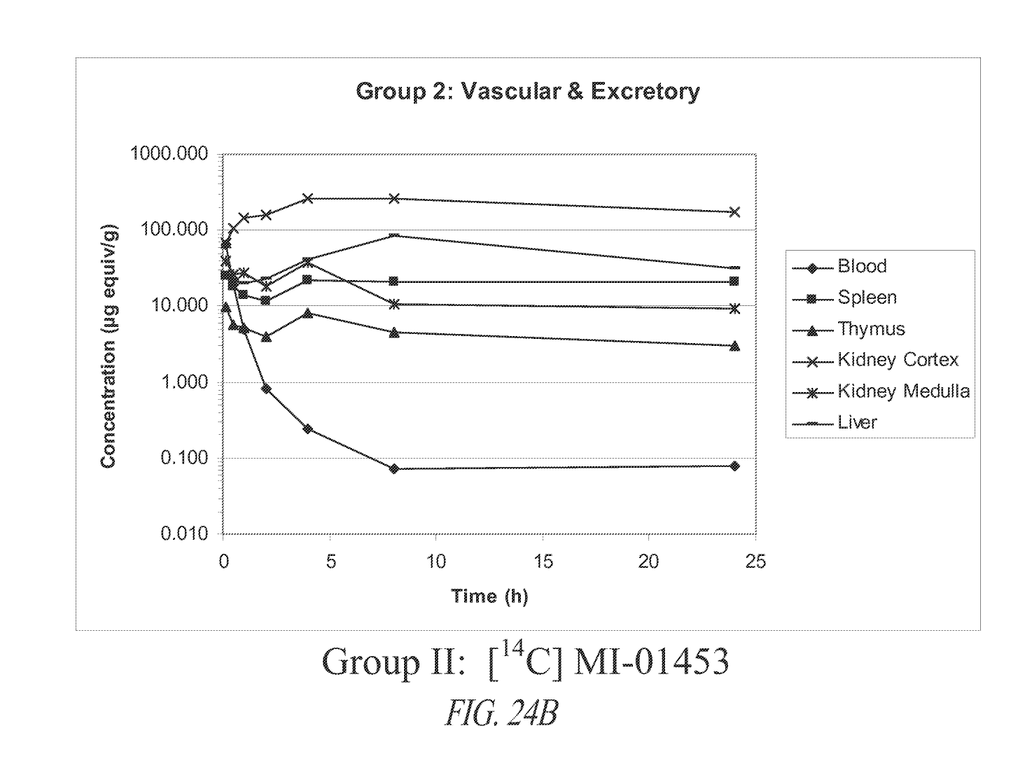

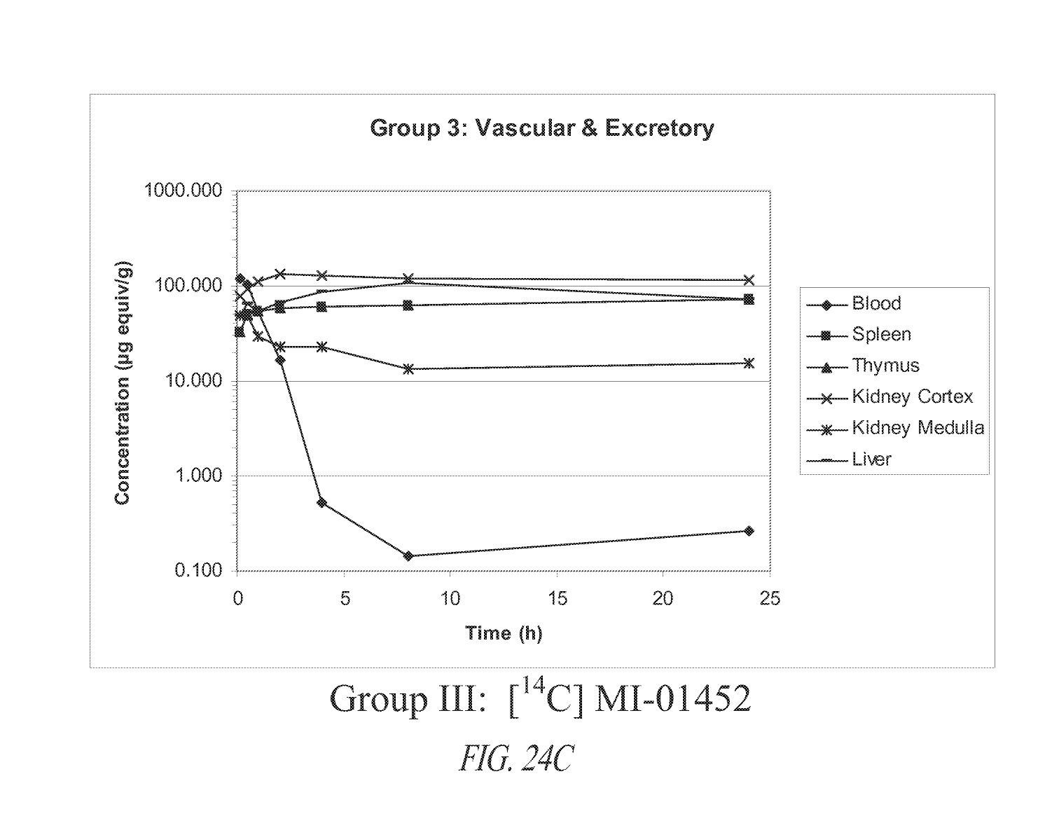

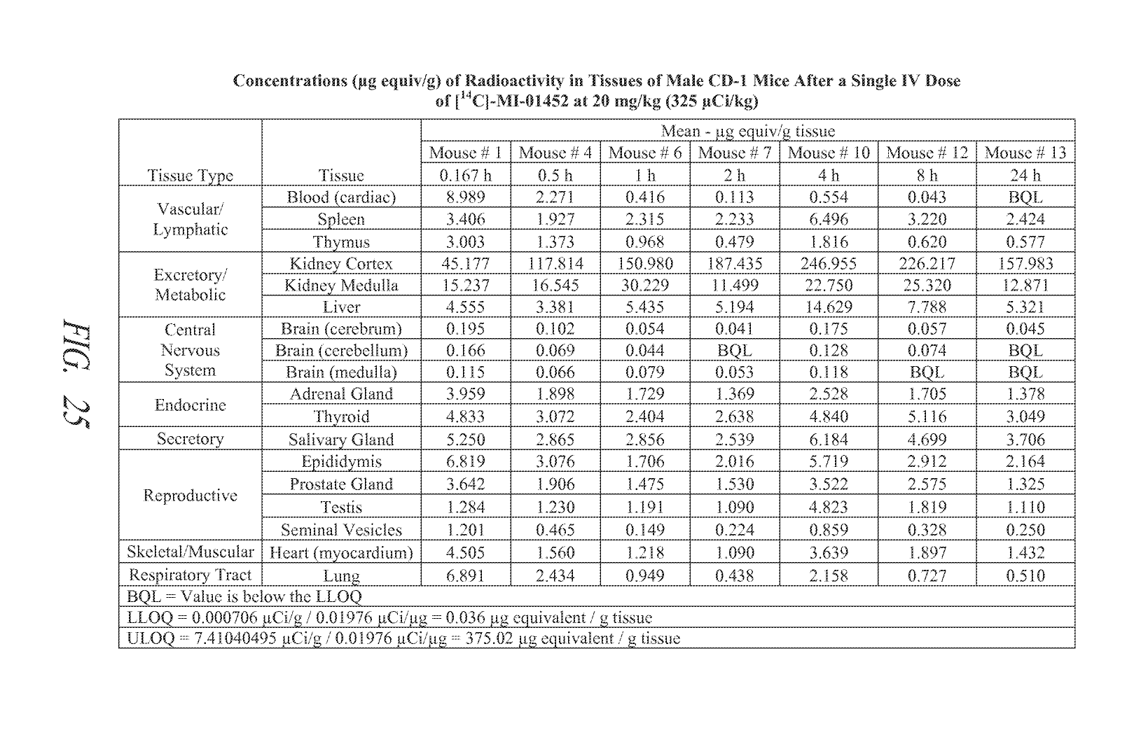

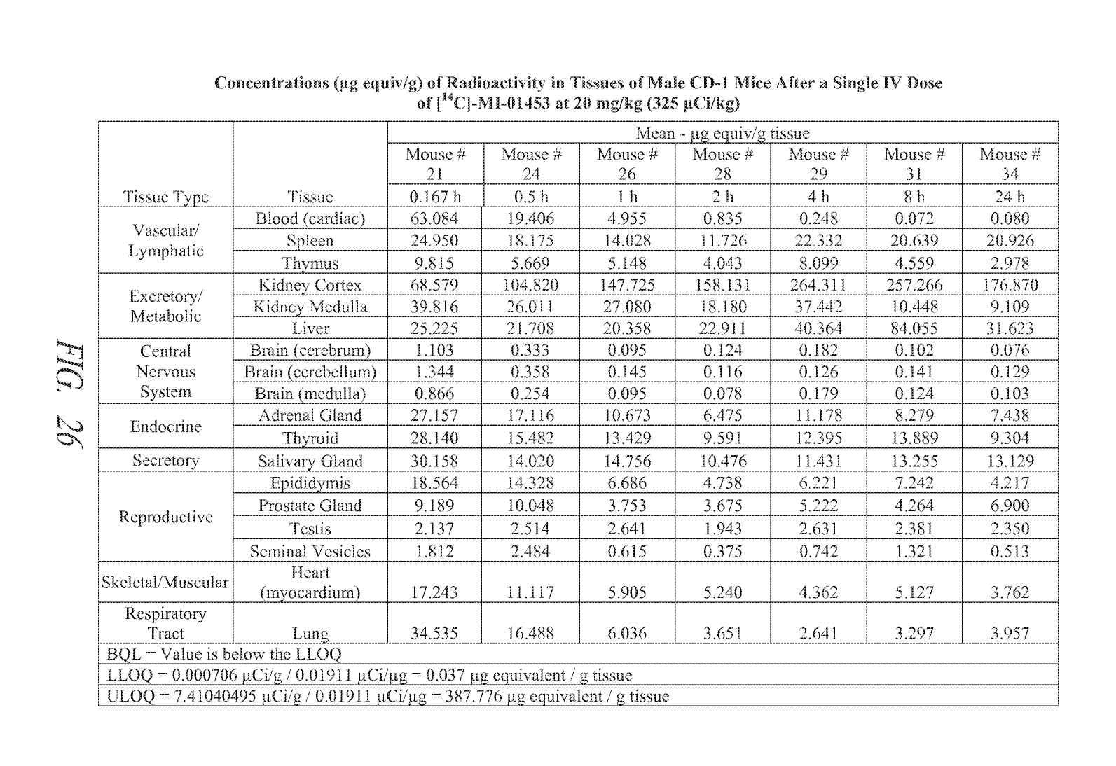

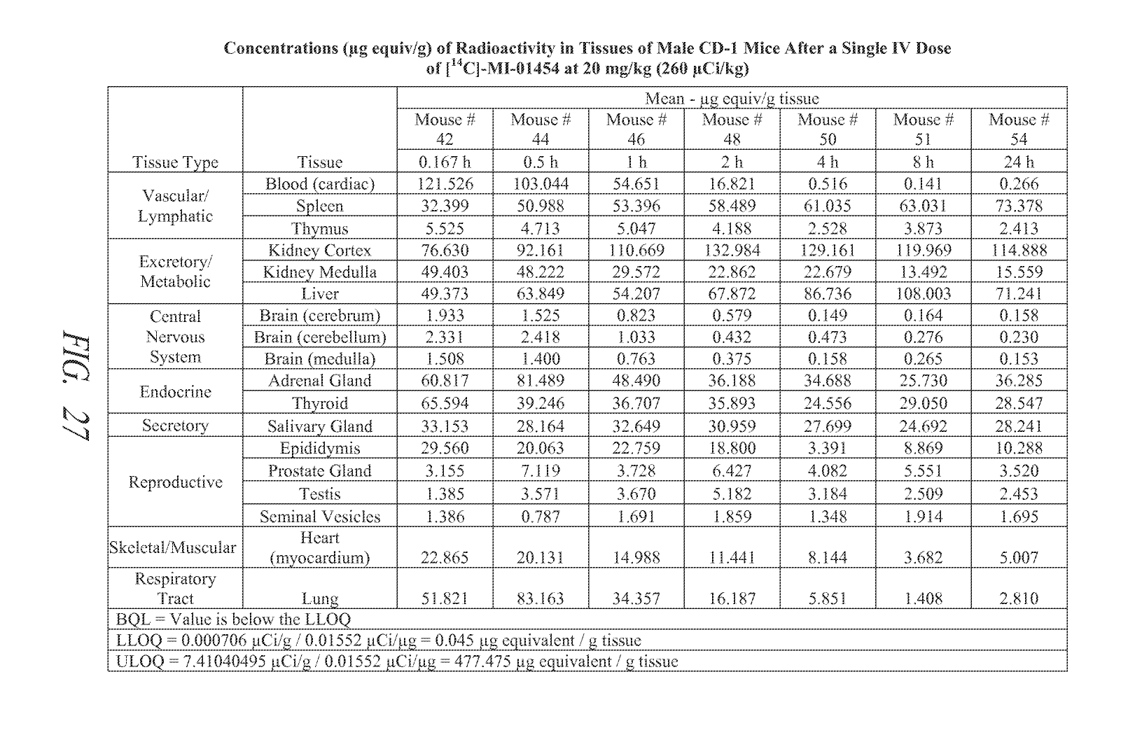

Whole-body autoradiograms showing patterns of radioactivity distribution in tissues are illustrated in FIGS. 1-21, graphs of tissue distribution are illustrated in FIGS. 22-24, and the concentrations of drug-derived radioactivity in the tissues of mice following IV administration of [¹⁴C] MI-01452, [¹⁴C] MI-01453, or [¹⁴C] MI-01454 are summarized in FIGS. 25-27.

1. [¹⁴C] MI-01452

Maximum concentrations (Cmax) of drug-derived radioactivity were observed at 0.167 h post-dose for most tissues (10 of 18 tissues measured). The highest concentrations of radioactivity at Cmax were observed in the kidney cortex (246.955 μg equiv/g, 4 h) and kidney medulla (30.229 μg equiv/g, 1 h). The concentrations in other tissues were substantially lower. The lowest tissue concentrations were measured in brain (<0.200 μg equiv/g) and seminal vesicles (<1.250 μg equiv/g). Most tissue concentrations decreased, but remained above the lower limits of quantitation ([¹⁴C]MI 01452: 0.036 μg equiv/g) at 24 h post-dose. The biodistribution of MI-01452 indicated that the unconjugated miRNAs primarily localized the kidney and renal excretion was the primary route of elimination. Elimination was not complete by the end of this study (24 h post-dose).

2. [¹⁴C] MI-01453

Maximum concentrations (Cmax) of drug-derived radioactivity were observed at 0.167 h post-dose for most tissues (13 of 18 tissues measured). High concentrations of MI-01453 were found in a wide variety of tissues. The highest concentrations of radioactivity at Cmax were in the following tissues: kidney cortex (264.311 μg equiv/g, 4 h), liver (84.055 μg equiv/g, 8 h), blood cardiac (63.084 μg equiv/g, 0.167 h), and kidney medulla (39.816 μg equiv/g, 0.167 h), lung (34.535 μg equiv/g, 0.167 h), and salivary gland (30.158 μg equiv/g, 0.167 h). Qualitatively the urinary bladder content, bone marrow, and some lymphatic tissue also appeared to have high concentrations. The lowest tissue concentrations were measured in brain and seminal vesicles. The biodistribution MI-01453 indicated that the conjugated anti-miRNAs are widely dispersed throughout the tissues and that renal excretion was the primary route of elimination. Most tissues had lower concentrations after the first sampled time point (0.167 h); however, concentrations in most tissues remained at a similar concentration at later time points through the 24 h time point. Elimination was not complete by the end of this study (24 h post-dose).

3. [¹⁴C] MI-01454

Maximum concentrations (Cmax) of drug-derived radioactivity were observed at 0.167 h post-dose for most tissues (8 of 18 tissues measured). High concentrations of MI-01454 were found in a wide variety of tissues. The highest concentrations of radioactivity at Cmax were in the following tissues: kidney cortex (132.984 μg equiv/g, 2 h), blood cardiac (121.526 μg equiv/g, 0.167 h), liver (108.003 μg equiv/g, 8 h), lung (83.163 μg equiv/g, 0.5 h), adrenal gland (81.489 μg equiv/g, 0.5 h), spleen (73.378 μg equiv/g, 24 h), thyroid (65.594 μg equiv/g, 0.167 h), kidney medulla (49.403 μg equiv/g, h), salivary gland (33.153 μg equiv/g, 0.167 h), and blood (121.526 μg equiv/g). Qualitatively the urinary bladder content, bone marrow, and some lymphatic tissue also appeared to have high concentrations. The lowest tissue concentrations were measured in brain (<2.500 μg equiv/g) and seminal vesicles (<2.000 μg equiv/g). The biodistribution MI-01454 indicated that the conjugated anti-miRNAs are widely dispersed throughout the tissues and that renal excretion was the primary route of elimination. Most tissues had lower concentrations after the first sampled time point (0.167 h); however, concentrations in most tissues remained at a similar concentration at later time points through the 24 h time point. Elimination was not complete by the end of this study (24 h post-dose).

### CONCLUSIONS

The biodistribution of conjugated anti-miRNAs was surprisingly and unexpectedly better than the limited distribution of unconjugated anti-miRNAs. High concentrations of drug-derived radioactivity were widely distributed to tissues throughout the study period for conjugated anti-miRNAs, e.g., MI-01453 and MI-01454, albeit at different concentrations for most tissues. In contrast, radioactivity of unconjugated anti-miRNAs, e.g., MI-01452, was not widely distributed: the unconjugated anti-miRNAs were primarily detected in the kidney, while some signal was also detected in the liver. Accordingly, conjugated miRNAs of the invention are advantageous because they are widely bioavailable; thus, solving the problem of limited bioavailability encountered with systemic administration of anti-miRNA therapeutics.

### ABBREVIATIONS

The following is a list of abbreviations used herein and their meanings: % CV=coefficient of variation; BQL=below the quantifiable limits; Cmax=maximal concentration observed; Ci=Curie; LLOQ=lower limit of quantification; LSC=liquid scintillation counting; MCID=micro-computer imaging device system; MDC=molecular dynamics counts; and ULOQ=upper limit of quantitation.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method of providing an oligomer to a plurality of cell types, tissues, or organs comprising administering to a subject, an oligomer comprising one or more minor groove binding moieties (MGBs).

2. A method of administering an oligomer to a plurality of cell types, tissues, or organs comprising administering to a subject, an oligomer comprising and one or more MGBs.

3. The method according to claim 1 or claim 2, wherein the oligomer is single stranded.

4. The method according to claim 1 or claim 2, wherein the oligomer is double stranded.

5. The method of claim 1 or claim 2, wherein the oligomer is selected from the group consisting of: an anti-miRNA, an siRNA, an shRNA, a piRNA mimetic, and a miRNA mimetic.

6. The method of claim 1 or claim 2, wherein the oligomer is parenterally administered.

7. The method according to claim 6, wherein the parenteral administration is selected from the group consisting of: intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

8. The method according to claim 1 or claim 2, wherein the oligomer is intravenously administered.

9. The method according to claim 1 or claim 2, wherein the plurality of cell types is selected from the group consisting of: cancer cells, immune cells, epithelial cells, endothelial cells, mesodermal cells, and mesenchymal cells, bone cells, hematopoietic cells, skin cells, hair cells, eye cells, neural cells, glial cells, muscle cells, meningeal cells, breast cells, liver cells, kidney cells, pancreatic cells, gastric cells, intestinal cells, colon cells, prostate cells, cervical cells, and vaginal cells.

10. The method according to claim 1 or claim 2, wherein the plurality of tissues is selected from the group consisting of: mesodermal tissue, connective tissue, smooth muscle tissue, striated muscle tissue, cardiac muscle tissue, bone tissue, bone marrow tissue, bone spongy tissue, cartilage tissue, adipose tissue, endodermal tissue, lung tissue, vascular tissue, pancreatic tissue, liver tissue, pancreatic ductal tissue, spleen tissue, thymus tissue, tonsil tissue, Peyer's patch tissue, lymph nodes tissue, thyroid tissue, endothelial tissue, blood cells, bladder tissue, kidney tissue, digestive tract tissue, esophagus tissue, stomach tissue, small intestine tissue, large intestine tissue, uterus tissue, testicular tissue, ovarian tissue, prostate tissue, endocrine tissue, mesentery tissue, and umbilical tissue, ectodermal tissue, epidermis tissue, dermis tissue, eye tissue, and nervous system tissue.

11. The method according to claim 1 or claim 2, wherein the plurality of organs is selected from the group consisting of: bladder, bone, brain, breast, cartilage, cervix, colon, cornea, eye, nervous tissue, glia, esophagus, fallopian tube, heart, pancreas, intestines, gallbladder, kidney, liver, lung, ovaries, pancreas, parathyroid gland, pineal gland, pituitary gland, prostate, spinal cord, spleen, skeletal muscle, skin, smooth muscle, stomach, testes, thymus, thyroid, trachea, urogenital tract, ureter, urethra, uterus, and vagina.

12. The method according to claim 1 or claim 2, wherein the oligomer hybridizes to a pre-mRNA.

13. The method according to claim 1 or claim 2, wherein the oligomer hybridizes to a target miRNA.

14. The method according to claim 1 or claim 2, wherein the anti-miRNA molecule hybridizes to a pre-mRNA.

15. The method according to claim 1 or claim 2, wherein the oligomer hybridizes to a target pri-miRNA.

16. The method according to claim 1 or claim 2, wherein at least one of the one or more MGBs is conjugated to the 5′ end or the 3′ end of the oligomer.

17. The method of claim 16, wherein at least one of the one or more MGBs is conjugated to the oligomer with a linker.

18. The method of claim 17, wherein the linker comprises a chain of about 10 to about 100 atoms selected from the group consisting of: C, O, N, S, and P.

19. The method of claim 18, wherein the linker is selected from the group consisting of:
a) —P(═O)(OH)O(CH₂)₆NH—;
b) —P(═O)(OH)O(CH₂)₄NH—;
c) —P(═O)(OH)(OCH₂CH₂)₆OP(═O)(OH)O(CH₂)₆NH—;
d) hydroxy{[5-(hydroxymethyl)-1-methylpyrrolidin-3 yl]oxy}oxophosphonium; and
e) —(CH₂)₅OP(═O)(OH)—.

20. The method according to claim 1 or claim 2, wherein at least one of the one or more MGBs selected from the group consisting of: netropsin, distamycin and lexitropsin, mithramycin, chromomycin A₃, olivomycin, anthramycin, sibiromycin, 1,2-dihydro-3H-pyrrolo[3,2-e)indole-7-carboxylic acid (DPI)₍₁₋₁₀₎, N3 carbamoyl 1,2-dihydro-3H-pyrrolo[3,2-e)indole-7-carboxylic acid (CDPI)₍₁₋₁₀₎, and N-methylpyrrole-4-carbox-2-amide (MPC)₍₁₋₁₀₎.

21. The method according to claim 1 or claim 2, wherein at least one of the one or more MGBs is CDPI₃ or CDPI₄.

22. The method according to claim 1 or claim 2, wherein at least one of the one or more MGBs is CDPI₃.

23. The method according to claim 1 or claim 2, wherein the oligomer comprises 6 to 100 nucleotides.

24. The method according to claim 1 or claim 2, wherein the oligomer comprises 10 to 50 nucleotides.

25. The method according to claim 1 or claim 2, wherein the oligomer comprises 15 to 23 nucleotides.

26. The method according to claim 1 or claim 2, wherein the oligomer comprises a nucleotide sequence that is at least 70% complementary to a target RNA sequence.

27. The method of claim 20, wherein the nucleotides are selected from the group of deoxyribonucleotides, ribonucleotides, and modified nucleotides.

28. The method of claim 22, wherein the modified nucleotides comprise a base selected from the groups consisting of: 5-methylcytosine, 5-hydroxymethyl cytosine, xanthine, hypoxanthine, 2-aminoadenine, 6-alkyladenine, 6-alkylguanine, 2-alkyladenine, 2-alkylguanine, 2-thiouracil, 2-thiothymine, 2-thiocytosine, 5-halouracil, 5-halocytosine, 5-alkynyluracil, 5-alkynylcytosine, 6-azo uracil, 6-azo cytosine, 6-azo thymine, 5-uracil, 4-thiouracil, 8-haloadenine, 8-aminoadenine, 8-thioladenine, 8-thioalkyladenine, 8-hydroxyladenine, 8-haloguanine, 8-aminoguanine, 8-thiolguanine, 8-thioalkylguanine, 8-hydroxylguanine, 5-halo uracil, 5-halo cytosine, 7-methylguanine, 7-methyladenine, 2-F-adenine, 2-amino-adenine, 8-azaguanine, 8azaadenine, 7-deazaguanine, 7-deazaadenine, 3-deazaguanine, 3-deazaadenine, phenoxazine cytidine, phenothiazine cytidine, G-clamp, carbazole cytidine, pyridoindole cytidine, 7-deaza adenine, 7-deaza guanosine, 2-aminopyridine, 2-pyridone, 2-aminopropyladenine, 5-propynyluracil, and 5-propynylcytosine.

29. The method according to claim 1 or claim 2, wherein the oligomer comprises at least one modified internucleoside linkage.

30. The method of claim 29, wherein the at least one modified internucleoside linkage is selected from the group consisting of: a phosphorothioate, phosphorodithioate, phosphotriester, aminoalkylphosphotriester, alkyl phosphonates, phosphinate, phosphoramidate, thionophosphoramidate, thionoalkylphosphonate, thionoalkylphosphotriester, selenophosphate, boranophosphate, morpholino, siloxane, sulfide, sulfoxide, sulfone, formacetyl, thioformacetyl, methylene formacetyl, riboacetyl, alkene-containing backbone, sulfamate, methyleneimino, methylenehydrazino, sulfonate, sulfonamide, or amide.

31. The method of claim 29, wherein the at least one modified internucleoside linkage is a phosphorothioate linkage.

32. The method of claim 29, wherein all of the internucleoside linkages of the oligomer are phosphorothioate linkages.

33. The method according to claim 1 or claim 2, wherein the oligomer comprises at least one 2′ modified sugar moiety.

34. The method of claim 33, wherein the at least one 2′ modified sugar moiety is selected from the group consisting of: OH, halogen, O-alkyl, S-alkyl, N-alkyl, O-alkenyl, S-alkenyl, N-alkenyl, O-alkynyl, S-alkynyl, N-alkynyl, O-alkyl-O-alkyl, alkaryl, aralkyl, O-alkaryl, O-aralkyl, SH, SCH₃, OCN, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, alkoxyalkoxy, dimethylaminooxyethoxy, allyl, and O-allyl, wherein the alkyl, alkenyl and alkynyl can be substituted or unsubstituted C1 to C10 alkyl or C2 to C10 alkenyl and alkynyl.

35. The method of claim 33, wherein the at least one 2′ modified sugar moiety is 2′-O-(2-methoxyethyl) (2′-MOE) sugar moiety.

36. The method of claim 33, wherein the at least one 2′ modified sugar moiety comprises a 2′-O, 4′-C methylene bridge.

37. The method according to claim 1 or claim 2, wherein the oligomer comprises at least one or more bases comprising a 3′ lipophilic group.

38. The method of claim 37, wherein the 3′ lipophilic group is selected from the group consisting of: cholesterol; a bile acid; and a fatty acid.

39. A method of decreasing the expression of an RNA in one or more cells, tissues, or organs, comprising administering an oligomer comprising one or more MGBs to a subject, wherein the RNA expression in the subject is decreased in the one or more cells, tissues, or organs, compared to the RNA expression in another subject administered an oligomer that does not comprise an MGB.

40. A method of decreasing the miRNA activity of an miRNA in one or more cells, tissues, or organs, comprising administering an anti-miRNA molecule comprising one or more MGBs to a subject, wherein the miRNA activity in the subject is decreased in the one or more cells, tissues, or organs, compared to the miRNA activity in another subject administered an anti-miRNA molecule that does not comprise an MGB.

41. A method of treating a subject having a disease, disorder or condition associated with increased expression of one or more RNAs in a plurality of cell types, tissues, or organs comprising:
a) identifying one or more RNAs in a plurality of cell types, tissues, or organs having increased RNA expression in diseased cells, tissues, or organs, compared to RNA expression of the one or more RNAs in a normal cell; and
b) administering an oligomer comprising one or more MGBs that hybridizes to the one or more RNAs.

42. A method of treating a subject having a disease, disorder or condition associated with increased activity of one or more miRNAs in a plurality of cell types, tissues, or organs comprising:
a) identifying one or more miRNAs in a plurality of cell types, tissues, or organs having increased miRNA activity in diseased cells, tissues, or organs, compared to miRNA activity of the one or more miRNA in a normal cell; and
b) administering an anti-miRNA molecule comprising an oligomer and one or more MGBs that hybridizes to the one or more miRNAs.

43. The method of claim 41 or claim 42, wherein the disease, disorder or condition is selected from the groups consisting of: tumor mediated angiogenesis, cancer, inflammation, fibrotic diseases, auto-immune diseases, and hepatitis C infection-mediated diseases.

44. The method of claim 41 or claim 42, wherein the disease, disorder or condition is selected from the groups consisting of: lung cancer, hepatocellular cancer, gastrointestinal cancer, pancreatic cancer, glioblastoma, leukemia, lymphoma, cervical cancer, ovarian cancer, kidney cancer, bladder cancer, breast cancer, osteosarcoma, cancer of the central nervous system, colon cancer, colorectal cancer, gastric cancer, endometrial or uterine carcinoma, salivary gland carcinoma, papillary renal cell carcinoma, prostate cancer, vulval cancer, thyroid cancer, and head and neck cancer, and melanoma.

45. The method of claim 41 or claim 42, wherein the disease, disorder or condition is selected from the groups consisting of: autoimmune thyroid disease, including Grave's disease and Hashimoto's thyroiditis, rheumatoid arthritis, systemic lupus erythematosus (SLE), Sjogrens syndrome, immune thrombocytopenic purpura (ITP), multiple sclerosis (MS), myasthenia gravis (MG), psoriasis, scleroderma, and inflammatory bowel disease, including Crohn's disease and ulcerative colitis.

46. The method of claim 41 or claim 42, wherein the disease, disorder or condition Hepatitis C infection or a Hepatitis C infection-mediated disease.

47. The method of claim 41 or claim 42, wherein the disease, disorder or condition is selected from the groups consisting of: neovascularization, stroke, ischemia, and myocardial infarction.

48. The method of claim 42, wherein the miRNA is selected from the group consisting of: hsa-let-7a-1; hsa-let-7a-2; hsa-let-7a-3; hsa-let-7b; hsa-let-7c; hsa-let-7d; hsa-let-7e; hsa-let-7f-1; hsa-let-7f-2; hsa-let-7g; hsa-let-7i; hsa-mir-100; hsa-mir-101-1; hsa-mir-101-2; hsa-mir-103-1; hsa-mir-103-1-as; hsa-mir-103-2; hsa-mir-103-2-as; hsa-mir-105-1; hsa-mir-105-2; hsa-mir-106a; hsa-mir-106b; hsa-mir-107; hsa-mir-10a; hsa-mir-10b; hsa-mir-1-1; hsa-mir-1178; hsa-mir-1179; hsa-mir-1180; hsa-mir-1181; hsa-mir-1182; hsa-mir-1183; hsa-mir-1184-1; hsa-mir-1184-2; hsa-mir-1184-3; hsa-mir-1185-1; hsa-mir-1185-2; hsa-mir-1193; hsa-mir-1197; hsa-mir-1-2; hsa-mir-1200; hsa-mir-1202; hsa-mir-1203; hsa-mir-1204; hsa-mir-1205; hsa-mir-1206; hsa-mir-1207; hsa-mir-1208; hsa-mir-122; hsa-mir-1224; hsa-mir-1225; hsa-mir-1226; hsa-mir-1227; hsa-mir-1228; hsa-mir-1229; hsa-mir-1231; hsa-mir-1233-1; hsa-mir-1233-2; hsa-mir-1234; hsa-mir-1236; hsa-mir-1237; hsa-mir-1238; hsa-mir-124-1; hsa-mir-124-2; hsa-mir-1243; hsa-mir-124-3; hsa-mir-1244-1; hsa-mir-1244-2; hsa-mir-1244-3; hsa-mir-1245; hsa-mir-1246; hsa-mir-1247; hsa-mir-1248; hsa-mir-1249; hsa-mir-1250; hsa-mir-1251; hsa-mir-1252; hsa-mir-1253; hsa-mir-1254; hsa-mir-1255a; hsa-mir-1255b-1; hsa-mir-1255b-2; hsa-mir-1256; hsa-mir-1257; hsa-mir-1258; hsa-mir-125a; hsa-mir-125b-1; hsa-mir-125b-2; hsa-mir-126; hsa-mir-1260; hsa-mir-1260b; hsa-mir-1261; hsa-mir-1262; hsa-mir-1263; hsa-mir-1264; hsa-mir-1265; hsa-mir-1266; hsa-mir-1267; hsa-mir-1268; hsa-mir-1269; hsa-mir-127; hsa-mir-1270-1; hsa-mir-1270-2; hsa-mir-1271; hsa-mir-1272; hsa-mir-1273; hsa-mir-1273c; hsa-mir-1273d; hsa-mir-1274a; hsa-mir-1274b; hsa-mir-1275; hsa-mir-1276; hsa-mir-1277; hsa-mir-1278; hsa-mir-1279; hsa-mir-1280; hsa-mir-1281; hsa-mir-128-1; hsa-mir-1282; hsa-mir-128-2; hsa-mir-1283-1; hsa-mir-1283-2; hsa-mir-1284; hsa-mir-1285-1; hsa-mir-1285-2; hsa-mir-1286; hsa-mir-1287; hsa-mir-1288; hsa-mir-1289-1; hsa-mir-1289-2; hsa-mir-1290; hsa-mir-1291; hsa-mir-129-1; hsa-mir-1292; hsa-mir-129-2; hsa-mir-1293; hsa-mir-1294; hsa-mir-1295; hsa-mir-1296; hsa-mir-1297; hsa-mir-1298; hsa-mir-1299; hsa-mir-1301; hsa-mir-1302-1; hsa-mir-1302-10; hsa-mir-1302-11; hsa-mir-1302-2; hsa-mir-1302-3; hsa-mir-1302-4; hsa-mir-1302-5; hsa-mir-1302-6; hsa-mir-1302-7; hsa-mir-1302-8; hsa-mir-1302-9; hsa-mir-1303; hsa-mir-1304; hsa-mir-1305; hsa-mir-1306; hsa-mir-1307; hsa-mir-130a; hsa-mir-130b; hsa-mir-132; hsa-mir-1321; hsa-mir-1322; hsa-mir-1323; hsa-mir-1324; hsa-mir-133a-1; hsa-mir-133a-2; hsa-mir-133b; hsa-mir-134; hsa-mir-135a-1; hsa-mir-135a-2; hsa-mir-135b; hsa-mir-136; hsa-mir-137; hsa-mir-138-1; hsa-mir-138-2; hsa-mir-139; hsa-mir-140; hsa-mir-141; hsa-mir-142; hsa-mir-143; hsa-mir-144; hsa-mir-145; hsa-mir-1468; hsa-mir-1469; hsa-mir-146a; hsa-mir-146b; hsa-mir-147; hsa-mir-1470; hsa-mir-1471; hsa-mir-147b; hsa-mir-148a; hsa-mir-148b; hsa-mir-149; hsa-mir-150; hsa-mir-151; hsa-mir-152; hsa-mir-153-1; hsa-mir-153-2; hsa-mir-1537; hsa-mir-1538; hsa-mir-1539; hsa-mir-154; hsa-mir-155; hsa-mir-15a; hsa-mir-15b; hsa-mir-16-1; hsa-mir-16-2; hsa-mir-17; hsa-mir-181a-1; hsa-mir-181a-2; hsa-mir-181b-1; hsa-mir-181b-2; hsa-mir-181c; hsa-mir-181d; hsa-mir-182; hsa-mir-1825; hsa-mir-1827; hsa-mir-183; hsa-mir-184; hsa-mir-185; hsa-mir-186; hsa-mir-187; hsa-mir-188; hsa-mir-18a; hsa-mir-18b; hsa-mir-190; hsa-mir-1908; hsa-mir-1909; hsa-mir-190b; hsa-mir-191; hsa-mir-1910; hsa-mir-1911; hsa-mir-1912; hsa-mir-1913; hsa-mir-1914; hsa-mir-1915; hsa-mir-192; hsa-mir-193a; hsa-mir-193b; hsa-mir-194-1; hsa-mir-194-2; hsa-mir-195; hsa-mir-196a-1; hsa-mir-196a-2; hsa-mir-196b; hsa-mir-197; hsa-mir-1972-1; hsa-mir-1972-2; hsa-mir-1973; hsa-mir-1976; hsa-mir-198; hsa-mir-199a-1; hsa-mir-199a-2; hsa-mir-199b; hsa-mir-19a; hsa-mir-19b-1; hsa-mir-19b-2; hsa-mir-200a; hsa-mir-200b; hsa-mir-200c; hsa-mir-202; hsa-mir-203; hsa-mir-204; hsa-mir-205; hsa-mir-2052; hsa-mir-2053; hsa-mir-2054; hsa-mir-206; hsa-mir-208a; hsa-mir-208b; hsa-mir-20a; hsa-mir-20b; hsa-mir-21; hsa-mir-210; hsa-mir-211; hsa-mir-2110; hsa-mir-2113; hsa-mir-2114; hsa-mir-2115; hsa-mir-2116; hsa-mir-2117; hsa-mir-212; hsa-mir-214; hsa-mir-215; hsa-mir-216a; hsa-mir-216b; hsa-mir-217; hsa-mir-218-1; hsa-mir-218-2; hsa-mir-219-1; hsa-mir-219-2; hsa-mir-22; hsa-mir-221; hsa-mir-222; hsa-mir-223; hsa-mir-224; hsa-mir-2276; hsa-mir-2277; hsa-mir-2278; hsa-mir-2355; hsa-mir-23a; hsa-mir-23b; hsa-mir-23c; hsa-mir-24-1; hsa-mir-24-2; hsa-mir-25; hsa-mir-26a-1; hsa-mir-26a-2; hsa-mir-26b; hsa-mir-27a; hsa-mir-27b; hsa-mir-28; hsa-mir-2861; hsa-mir-2909; hsa-mir-296; hsa-mir-297; hsa-mir-298; hsa-mir-299; hsa-mir-29a; hsa-mir-29b-1; hsa-mir-29b-2; hsa-mir-29c; hsa-mir-300; hsa-mir-301a; hsa-mir-301 b; hsa-mir-302a; hsa-mir-302b; hsa-mir-302c; hsa-mir-302d; hsa-mir-302e; hsa-mir-302f; hsa-mir-3065; hsa-mir-3074; hsa-mir-30a; hsa-mir-30b; hsa-mir-30c-1; hsa-mir-30c-2; hsa-mir-30d; hsa-mir-30e; hsa-mir-31; hsa-mir-3115; hsa-mir-3116-1; hsa-mir-3116-2; hsa-mir-3117; hsa-mir-3118-1; hsa-mir-3118-2; hsa-mir-3118-3; hsa-mir-3118-4; hsa-mir-3118-5; hsa-mir-3118-6; hsa-mir-3119-1; hsa-mir-3119-2; hsa-mir-3120; hsa-mir-3121; hsa-mir-3122; hsa-mir-3123; hsa-mir-3124; hsa-mir-3125; hsa-mir-3126; hsa-mir-3127; hsa-mir-3128; hsa-mir-3129; hsa-mir-3130-1; hsa-mir-3130-2; hsa-mir-3131; hsa-mir-3132; hsa-mir-3133; hsa-mir-3134; hsa-mir-3135; hsa-mir-3136; hsa-mir-3137; hsa-mir-3138; hsa-mir-3139; hsa-mir-3140; hsa-mir-3141; hsa-mir-3142; hsa-mir-3143; hsa-mir-3144; hsa-mir-3145; hsa-mir-3146; hsa-mir-3147; hsa-mir-3148; hsa-mir-3149; hsa-mir-3150; hsa-mir-3150b; hsa-mir-3151; hsa-mir-3152; hsa-mir-3153; hsa-mir-3154; hsa-mir-3155; hsa-mir-3156-1; hsa-mir-3156-2; hsa-mir-3156-3; hsa-mir-3157; hsa-mir-3158-1; hsa-mir-3158-2; hsa-mir-3159; hsa-mir-3160-1; hsa-mir-3160-2; hsa-mir-3161; hsa-mir-3162; hsa-mir-3163; hsa-mir-3164; hsa-mir-3165; hsa-mir-3166; hsa-mir-3167; hsa-mir-3168; hsa-mir-3169; hsa-mir-3170; hsa-mir-3171; hsa-mir-3173; hsa-mir-3174; hsa-mir-3175; hsa-mir-3176; hsa-mir-3177; hsa-mir-3178; hsa-mir-3179-1; hsa-mir-3179-2; hsa-mir-3179-3; hsa-mir-3180-1; hsa-mir-3180-2; hsa-mir-3180-3; hsa-mir-3180-4; hsa-mir-3180-5; hsa-mir-3181; hsa-mir-3182; hsa-mir-3183; hsa-mir-3184; hsa-mir-3185; hsa-mir-3186; hsa-mir-3187; hsa-mir-3188; hsa-mir-3189; hsa-mir-3190; hsa-mir-3191; hsa-mir-3192; hsa-mir-3193; hsa-mir-3194; hsa-mir-3195; hsa-mir-3196; hsa-mir-3197; hsa-mir-3198; hsa-mir-3199-1; hsa-mir-3199-2; hsa-mir-32; hsa-mir-3200; hsa-mir-3201; hsa-mir-3202-1; hsa-mir-3202-2; hsa-mir-320a; hsa-mir-320b-1; hsa-mir-320b-2; hsa-mir-320c-1; hsa-mir-320c-2; hsa-mir-320d-1; hsa-mir-320d-2; hsa-mir-320e; hsa-mir-323; hsa-mir-323b; hsa-mir-324; hsa-mir-325; hsa-mir-326; hsa-mir-328; hsa-mir-329-1; hsa-mir-329-2; hsa-mir-330; hsa-mir-331; hsa-mir-335; hsa-mir-337; hsa-mir-338; hsa-mir-339; hsa-mir-33a; hsa-mir-33b; hsa-mir-340; hsa-mir-342; hsa-mir-345; hsa-mir-346; hsa-mir-34a; hsa-mir-34b; hsa-mir-34c; hsa-mir-3605; hsa-mir-3606; hsa-mir-3607; hsa-mir-3609; hsa-mir-361; hsa-mir-3610; hsa-mir-3611; hsa-mir-3612; hsa-mir-3613; hsa-mir-3614; hsa-mir-3615; hsa-mir-3616; hsa-mir-3617; hsa-mir-3618; hsa-mir-3619; hsa-mir-362; hsa-mir-3620; hsa-mir-3621; hsa-mir-3622a; hsa-mir-3622b; hsa-mir-363; hsa-mir-3646; hsa-mir-3647; hsa-mir-3648; hsa-mir-3649; hsa-mir-3650; hsa-mir-3651; hsa-mir-365-1; hsa-mir-3652; hsa-mir-365-2; hsa-mir-3653; hsa-mir-3654; hsa-mir-3655; hsa-mir-3656; hsa-mir-3657; hsa-mir-3658; hsa-mir-3659; hsa-mir-3660; hsa-mir-3661; hsa-mir-3662; hsa-mir-3663; hsa-mir-3664; hsa-mir-3665; hsa-mir-3666; hsa-mir-3667; hsa-mir-3668; hsa-mir-3669; hsa-mir-367; hsa-mir-3670; hsa-mir-3670; hsa-mir-3670; hsa-mir-3671; hsa-mir-3672; hsa-mir-3673; hsa-mir-3674; hsa-mir-3675; hsa-mir-3676; hsa-mir-3677; hsa-mir-3678; hsa-mir-3679; hsa-mir-3680; hsa-mir-3680; hsa-mir-3681; hsa-mir-3682; hsa-mir-3683; hsa-mir-3684; hsa-mir-3685; hsa-mir-3686; hsa-mir-3687; hsa-mir-3688; hsa-mir-3689a; hsa-mir-3689b; hsa-mir-369; hsa-mir-3690; hsa-mir-3691; hsa-mir-3692; hsa-mir-370; hsa-mir-371; hsa-mir-3713; hsa-mir-3714; hsa-mir-372; hsa-mir-373; hsa-mir-374a; hsa-mir-374b; hsa-mir-374c; hsa-mir-375; hsa-mir-376a-1; hsa-mir-376a-2; hsa-mir-376b; hsa-mir-376c; hsa-mir-377; hsa-mir-378; hsa-mir-378b; hsa-mir-378c; hsa-mir-379; hsa-mir-380; hsa-mir-381; hsa-mir-382; hsa-mir-383; hsa-mir-384; hsa-mir-3907; hsa-mir-3908; hsa-mir-3909; hsa-mir-3910-1; hsa-mir-3910-2; hsa-mir-3911; hsa-mir-3912; hsa-mir-3913-1; hsa-mir-3913-2; hsa-mir-3914-1; hsa-mir-3914-2; hsa-mir-3915; hsa-mir-3916; hsa-mir-3917; hsa-mir-3918; hsa-mir-3919; hsa-mir-3920; hsa-mir-3921; hsa-mir-3922; hsa-mir-3923; hsa-mir-3924; hsa-mir-3925; hsa-mir-3926-1; hsa-mir-3926-2; hsa-mir-3927; hsa-mir-3928; hsa-mir-3929; hsa-mir-3934; hsa-mir-3935; hsa-mir-3936; hsa-mir-3937; hsa-mir-3938; hsa-mir-3939; hsa-mir-3940; hsa-mir-3941; hsa-mir-3942; hsa-mir-3943; hsa-mir-3944; hsa-mir-3945; hsa-mir-409; hsa-mir-410; hsa-mir-411; hsa-mir-412; hsa-mir-421; hsa-mir-422a; hsa-mir-423; hsa-mir-424; hsa-mir-425; hsa-mir-4251; hsa-mir-4252; hsa-mir-4253; hsa-mir-4254; hsa-mir-4255; hsa-mir-4256; hsa-mir-4257; hsa-mir-4258; hsa-mir-4259; hsa-mir-4260; hsa-mir-4261; hsa-mir-4262; hsa-mir-4263; hsa-mir-4264; hsa-mir-4265; hsa-mir-4266; hsa-mir-4267; hsa-mir-4268; hsa-mir-4269; hsa-mir-4270; hsa-mir-4271; hsa-mir-4272; hsa-mir-4273; hsa-mir-4274; hsa-mir-4275; hsa-mir-4276; hsa-mir-4277; hsa-mir-4278; hsa-mir-4279; hsa-mir-4280; hsa-mir-4281; hsa-mir-4282; hsa-mir-4283-1; hsa-mir-4283-2; hsa-mir-4284; hsa-mir-4285; hsa-mir-4286; hsa-mir-4287; hsa-mir-4288; hsa-mir-4289; hsa-mir-429; hsa-mir-4290; hsa-mir-4291; hsa-mir-4292; hsa-mir-4293; hsa-mir-4294; hsa-mir-4295; hsa-mir-4296; hsa-mir-4297; hsa-mir-4298; hsa-mir-4299; hsa-mir-4300; hsa-mir-4301; hsa-mir-4302; hsa-mir-4303; hsa-mir-4304; hsa-mir-4305; hsa-mir-4306; hsa-mir-4307; hsa-mir-4308; hsa-mir-4309; hsa-mir-431; hsa-mir-4310; hsa-mir-4311; hsa-mir-4312; hsa-mir-4313; hsa-mir-4314; hsa-mir-4315-1; hsa-mir-4315-2; hsa-mir-4316; hsa-mir-4317; hsa-mir-4318; hsa-mir-4319; hsa-mir-432; hsa-mir-4320; hsa-mir-4321; hsa-mir-4322; hsa-mir-4323; hsa-mir-4324; hsa-mir-4325; hsa-mir-4326; hsa-mir-4327; hsa-mir-4328; hsa-mir-4329; hsa-mir-433; hsa-mir-4330; hsa-mir-448; hsa-mir-449a; hsa-mir-449b; hsa-mir-449c; hsa-mir-450a-1; hsa-mir-450a-2; hsa-mir-450b; hsa-mir-451; hsa-mir-452; hsa-mir-454; hsa-mir-455; hsa-mir-466; hsa-mir-483; hsa-mir-484; hsa-mir-485; hsa-mir-486; hsa-mir-487a; hsa-mir-487b; hsa-mir-488; hsa-mir-489; hsa-mir-490; hsa-mir-491; hsa-mir-492; hsa-mir-493; hsa-mir-494; hsa-mir-495; hsa-mir-496; hsa-mir-497; hsa-mir-498; hsa-mir-499; hsa-mir-500a; hsa-mir-500b; hsa-mir-501; hsa-mir-502; hsa-mir-503; hsa-mir-504; hsa-mir-505; hsa-mir-506; hsa-mir-507; hsa-mir-508; hsa-mir-509-1; hsa-mir-509-2; hsa-mir-509-3; hsa-mir-510; hsa-mir-511-1; hsa-mir-511-2; hsa-mir-512-1; hsa-mir-512-2; hsa-mir-513a-1; hsa-mir-513a-2; hsa-mir-513b; hsa-mir-513c; hsa-mir-514-1; hsa-mir-514-2; hsa-mir-514-3; hsa-mir-514b; hsa-mir-515-1; hsa-mir-515-2; hsa-mir-516a-1; hsa-mir-516a-2; hsa-mir-516b-1; hsa-mir-516b-2; hsa-mir-517a; hsa-mir-517b; hsa-mir-517c; hsa-mir-518a-1; hsa-mir-518a-2; hsa-mir-518b; hsa-mir-518c; hsa-mir-518d; hsa-mir-518e; hsa-mir-518f; hsa-mir-519a-1; hsa-mir-519a-2; hsa-mir-519b; hsa-mir-519c; hsa-mir-519d; hsa-mir-519e; hsa-mir-520a; hsa-mir-520b; hsa-mir-520c; hsa-mir-520d; hsa-mir-520e; hsa-mir-520f; hsa-mir-520g; hsa-mir-520h; hsa-mir-521-1; hsa-mir-521-2; hsa-mir-522; hsa-mir-523; hsa-mir-524; hsa-mir-525; hsa-mir-526a-1; hsa-mir-526a-2; hsa-mir-526b; hsa-mir-527; hsa-mir-532; hsa-mir-539; hsa-mir-541; hsa-mir-542; hsa-mir-543; hsa-mir-544; hsa-mir-544b; hsa-mir-545; hsa-mir-548a-1; hsa-mir-548a-2; hsa-mir-548a-3; hsa-mir-548aa-1; hsa-mir-548aa-2; hsa-mir-548b; hsa-mir-548c; hsa-mir-548d-1; hsa-mir-548d-2; hsa-mir-548e; hsa-mir-548f-1; hsa-mir-548f-2; hsa-mir-548f-3; hsa-mir-548f-4; hsa-mir-548f-5; hsa-mir-548g; hsa-mir-548h-1; hsa-mir-548h-2; hsa-mir-548h-3; hsa-mir-548h-4; hsa-mir-548i-1; hsa-mir-548i-2; hsa-mir-548i-3; hsa-mir-548i-4; hsa-mir-548j; hsa-mir-548k; hsa-mir-5481; hsa-mir-548m; hsa-mir-548n; hsa-mir-548o; hsa-mir-548p; hsa-mir-548q; hsa-mir-548s; hsa-mir-548t; hsa-mir-548u; hsa-mir-548v; hsa-mir-548w; hsa-mir-548x; hsa-mir-548y; hsa-mir-548z; hsa-mir-549; hsa-mir-550a-1; hsa-mir-550a-2; hsa-mir-550b-1; hsa-mir-550b-2; hsa-mir-551a; hsa-mir-551 b; hsa-mir-552; hsa-mir-553; hsa-mir-554; hsa-mir-555; hsa-mir-556; hsa-mir-557; hsa-mir-558; hsa-mir-559; hsa-mir-561; hsa-mir-562; hsa-mir-563; hsa-mir-564; hsa-mir-566; hsa-mir-567; hsa-mir-568; hsa-mir-569; hsa-mir-570; hsa-mir-571; hsa-mir-572; hsa-mir-573; hsa-mir-574; hsa-mir-575; hsa-mir-576; hsa-mir-577; hsa-mir-578; hsa-mir-579; hsa-mir-580; hsa-mir-581; hsa-mir-582; hsa-mir-583; hsa-mir-584; hsa-mir-585; hsa-mir-586; hsa-mir-587; hsa-mir-588; hsa-mir-589; hsa-mir-590; hsa-mir-591; hsa-mir-592; hsa-mir-593; hsa-mir-595; hsa-mir-596; hsa-mir-597; hsa-mir-598; hsa-mir-599; hsa-mir-600; hsa-mir-601; hsa-mir-602; hsa-mir-603; hsa-mir-604; hsa-mir-605; hsa-mir-606; hsa-mir-607; hsa-mir-608; hsa-mir-609; hsa-mir-610; hsa-mir-611; hsa-mir-612; hsa-mir-613; hsa-mir-614; hsa-mir-615; hsa-mir-616; hsa-mir-617; hsa-mir-618; hsa-mir-619; hsa-mir-620; hsa-mir-621; hsa-mir-622; hsa-mir-623; hsa-mir-624; hsa-mir-625; hsa-mir-626; hsa-mir-627; hsa-mir-628; hsa-mir-629; hsa-mir-630; hsa-mir-631; hsa-mir-632; hsa-mir-633; hsa-mir-634; hsa-mir-635; hsa-mir-636; hsa-mir-637; hsa-mir-638; hsa-mir-639; hsa-mir-640; hsa-mir-641; hsa-mir-642a; hsa-mir-642b; hsa-mir-643; hsa-mir-644; hsa-mir-645; hsa-mir-646; hsa-mir-647; hsa-mir-648; hsa-mir-649; hsa-mir-650; hsa-mir-651; hsa-mir-652; hsa-mir-653; hsa-mir-654; hsa-mir-655; hsa-mir-656; hsa-mir-657; hsa-mir-658; hsa-mir-659; hsa-mir-660; hsa-mir-661; hsa-mir-662; hsa-mir-663; hsa-mir-663b; hsa-mir-664; hsa-mir-665; hsa-mir-668; hsa-mir-670; hsa-mir-671; hsa-mir-675; hsa-mir-676; hsa-mir-708; hsa-mir-7-1; hsa-mir-711; hsa-mir-718; hsa-mir-7-2; hsa-mir-720; hsa-mir-7-3; hsa-mir-744; hsa-mir-758; hsa-mir-759; hsa-mir-760; hsa-mir-761; hsa-mir-762; hsa-mir-764; hsa-mir-765; hsa-mir-766; hsa-mir-767; hsa-mir-769; hsa-mir-770; hsa-mir-802; hsa-mir-873; hsa-mir-874; hsa-mir-875; hsa-mir-876; hsa-mir-877; hsa-mir-885; hsa-mir-887; hsa-mir-888; hsa-mir-889; hsa-mir-890; hsa-mir-891a; hsa-mir-891b; hsa-mir-892a; hsa-mir-892b; hsa-mir-9-1; hsa-mir-9-2; hsa-mir-920; hsa-mir-921; hsa-mir-922; hsa-mir-924; hsa-mir-92a-1; hsa-mir-92a-2; hsa-mir-92b; hsa-mir-93; hsa-mir-9-3; hsa-mir-933; hsa-mir-934; hsa-mir-935; hsa-mir-936; hsa-mir-937; hsa-mir-938; hsa-mir-939; hsa-mir-940; hsa-mir-941-1; hsa-mir-941-3; hsa-mir-941-4; hsa-mir-942; hsa-mir-943; hsa-mir-944; hsa-mir-95; hsa-mir-96; hsa-mir-98; hsa-mir-99a; hsa-mir-99b.ebv-miR-BHRFI-1, ebv-miR-BHRFI-2*, ebv-miR-BHRFI-2, ebv-miR-BHRFI-3, ebv-miR-BARTI-5p, ebv-miR-BART2, hcmv-miR-UL22A, hcmv-miR-UL22A*, hcmv-miR-UL36, hcmv-miR-UL112, hcmv-miR-UL148D, hcmv-miR-US5-1, hcmv-miR-US5-2, hcmv-miR-US25-I, hcmv-miR-US25-2-5p, hcmv-miR-US25-2-3p, hcmv-miR-US33, kshv-miR-K12-10a, kshv-miR-K12-10b, kshv-miR-K12-II, kshv-miR-K12-I, kshv-miR-K12-2, kshv-miR-K12-9*, kshv-miR-K12-9, kshv-miR-K12-8, kshv-miR-K12-7, kshv-miR-K12-6-5p, kshv-miR-K12-6-3p, kshv-miR-K12-5, kshv-miR-K12-4-5p, kshv-miR-K12-4-3p, kshv-miR-K12-3, kshv-miR-K12-3*, hcmv-miR-US4, hcmv-miR-UL70-5p, hcmv-20 miR-UL70-3p, ebv-miR-BART3-5p, ebv-miR-BART3-3p, ebv-miR-BART4, ebv-miR-BART5, ebv-miR-BART6-5p, ebv-miR-BART6-3p, ebv-miR-BART7, ebv-miR-BART8-5p, ebv-miR-BART8-3p, ebv-miR-BART9, ebv-miR-BARTIO, ebv-miR-BARTII-5p, ebv-miR-BARTII-3p, ebv-miR-BART12, ebv-miR-BART13, ebv-miR-BART14-5p, ebv-miR-BART14-3p, kshv-miR-25 K12-12, ebv-miR-BART15, ebv-miR-BART16, ebv-miR-BART17-5p, ebv-miR-BART17-3p, ebv-miR-BART18, ebv-miR-BART19, ebv-miR-BART20-5p, ebv-miR-BART20-3p, and hsvl-miR-HI.


SEQUENCE LISTING (3 sequences)

SEQ ID NO: 1 - RNA, 22 nt, Artificial Sequence
  Anti micro RNA oligonucleotide
ucaacaucagucugauaagcua

SEQ ID NO: 2 - RNA, 22 nt, Artificial Sequence
  Anti micro RNA oligonucleotide
ucaacaucagucugauaagcua

SEQ ID NO: 3 - RNA, 22 nt, Artificial Sequence
  Anti micro RNA oligonucleotide
ucaacaucagucugauaagcua